(19) United States
(12) Patent Application Publication
(10) Pub. No.: US 2025/0101466 A1
(43) Pub. Date: Mar. 27, 2025

(54) METHOD OF EDITING NUCLEIC ACID

(71) Applicant: Academisch Ziekenhuis Leiden a/u Leiden University Medical Center, Leiden (NL)

(72) Inventors: Zhihan ZHAO, Leiden (NL); Peng SHANG, Leiden (NL); Niels GEIJSEN, Leiden (NL)

(21) Appl. No.: 18/715,550

(22) PCT Filed: Dec. 2, 2022
(86) PCT No.: PCT/EP2022/084232

§ 371 Date: May 31, 2024

(30) Foreign Application Priority Data
Dec. 2, 2021 (GB) 2117455.2

(51) Int. Cl. C12N 15/90; C12N 9/22; C12N 15/11

(52) U.S. Cl. CPC C12N 15/907; C12N 9/22; C12N 15/11; C12N 2310/20; C12N 2800/80

(57) ABSTRACT

The present disclosure provides efficient and precise methods and tools for use in the editing of nucleic acid. The disclosure provides a method of editing a nucleic acid comprising cutting or cleaving a nucleic acid to be edited, contacting the cut nucleic acid with a nucleic acid repair template so that the nucleic acid repair template comprises an end which matches a cut end of the nucleic acid to be edited and a sequence that is homologous to a sequence of nucleic acid to be edited.

### FIELD

The present disclosure provides methods and tools with application in the editing of nucleic acid. The methods or tools can be used in genome editing and are efficient and precise.

### BACKGROUND

Over the past decade, genome editing technologies have become one of the essential molecular techniques in biomedical research (1-4). Given that many human diseases have a genetic basis, these genome editing technologies, especially precise insertion, deletion, or replacement of parts of the genome, hold tremendous promise for the treatment of monogenetic disorders (5-8). Since their discovery, CRISPR/Cas systems have quickly become the editing technology of choice for targeted genome manipulation (9-12). CRISPR/Cas systems employ a short RNA molecule, the guide RNA, to lead a CRISPR effector nuclease to the genomic target position of interest, creating a DSB at the genomic target site. Subsequently, DNA damage-induced endogenous DNA repair machineries are recruited towards the cut site and repair the damage, by which the genomic manipulations occur and the ‘editing’ is finalized (13,14).

Cas12a (previously known as Cpf1), like Cas9, is a single-effector CRISPR protein (15,16). Cas12a differs from Cas9 with respect to several important properties (17). Cas12a naturally employs a single CRISPR RNA (crRNA) as guide RNA, which is substantially shorter than the engineered single guide RNA (sgRNA) used for Cas9 (18). In addition, Cas12a recognizes a T-rich protospacer adjacent motif (PAM) sequence, comparing to the G-rich PAM recognized by Cas9 (16). And importantly, Cas12a uses a single RuvC catalytic domain to cleave both the target and non-target strands, creating 5′-overhang sticky ends (19,20), while Cas9 employs two nuclease domains RuvC and HNH generating a blunt-ended DSB at the target locus (21,22).

Some of these unique properties of Cas12a have been exploited in various practical applications, for example, to edit AT-rich sequences that lack adequate Cas9 target sites (23,24). Other unique applications include: Cas12a's crRNA processing ability was utilized to simplify multiplex genome editing (25); DNase-dead Cas12a-carried base-editing domains achieved base-editing without DNA strand breaks (26,27); the on-target binding-induced collateral ssDNA cleavage activity was further developed to a virus detect tool in clinical diagnostics (28); and the ability to generate sticky-ends at the target DNA cleavage site has been employed as a tool for in vitro DNA assembly (29,30). Recently, Li et al developed a new Cas12a-based genome editing method named MITI (microhomology-dependent targeted integration) (31), which utilized the Cas12a-generated compatible sticky ends between transgene and target site termini to direct a site-specific gene insertion. The authors demonstrated how MITI could be applied to insert a gene of interest together with a positive selection cassette into a single Cas12a target-site in the genome. Yet, the need for a selection cassette and the reported inaccurate integration of the targeting construct at the 5′ and 3′ junctions restrain its application, especially in the context of gene therapeutics.

Two AsCas12a cleavage sites on a genome may be used to excise a target sequence and replace it with a dsDNA insert containing compatible sticky ends. This strategy has been termed ‘Cut-And-Paste Repair’ (CAPR), but the repair efficiency is rather low.

There is a need to continue to expand the repertoire of methods and tools for the efficient and precise editing of nuclei acid/genome sequences.

### SUMMARY

The present invention is based on the finding that nucleic acids (including genomic nucleic acids) can be efficiently altered or edited using methods which exploit both ‘sticky-ended’ ligation (through microhomology-mediated end joining) and homology directed repair (HDR). The invention is further based on the finding that repair templates which comprise a single sticky end compatible with the 5′ overhang created by certain nucleases (including, for example nucleases of the CRISPR-Cas system), allow sticky-ended ligation, and may be sufficient to effect efficient nucleic acid/genome editing.

Throughout this specification, the terms “comprise”, “comprising” and/or “comprises” is/are used to denote aspects and embodiments of this invention that “comprise” a particular feature or features. It should be understood that this/these terms may also encompass aspects and/or embodiments which “consist essentially of” or “consist of” the relevant feature or features.

For convenience, the various methods of this disclosure shall generally be referred to as “methods of editing nucleic acids”.

The term “editing” should be taken to embrace the act of altering a nucleic acid sequence—that is making one or more changes to a nucleic acid sequence. In this regard, a method of editing a nucleic acid sequence may be used to correct or introduce a mutation and or to introduce a particular sequence (to another). A method of editing a nucleic acid sequence may comprise replacing one sequence (or part(s) of a sequence) with another. A method of editing as described herein may focus on making one or more predetermined or defined changes to the nucleic acid sequence of a specific target region. The term ‘target region’ should be taken to mean a part or portion of a nucleic acid sequence which comprises a sequence which is to be edited. The term ‘editing’ as applied to a nucleic acid sequence may embrace any alteration or modification of the sequence of a target region. For example, an editing method of this disclosure may comprise correcting an error within a target region. In another example, an editing method of this disclosure may comprise altering (e.g. changing) one or more of the nucleobases within a target region. In a further example, an editing method of this disclosure may be used to add nucleobases to a target region. Additionally or alternatively, an editing method of this disclosure may be used to delete nucleobases from a target region within a nucleic acid sequence.

Within the context of this disclosure, a nucleic acid to be edited may be a genomic nucleic acid. As such, the phrase “nucleic acid editing” may embrace a method of genome editing. The nucleic acid to be edited may be a synthetic or isolated nucleic acid sequence from any source. The nucleic acid to be edited may comprise an exogenous sequence. The nucleic acid to be edited may comprise an endogenous sequence. The nucleic acid sequence to be edited may be part of a genome. The nucleic acid to be edited may be a double stranded nucleic acid.

As such, the disclosure provides a method of editing a nucleic acid, said method comprising:
cutting or cleaving the nucleic acid to be edited; and
contacting the cut nucleic acid with a nucleic acid repair template;
wherein the nucleic acid repair template comprises an end which matches a cut end of the nucleic acid to be edited and a sequence that is homologous to a sequence of nucleic acid to be edited.

The disclosure further provides a repair template for use in a genome editing method or in a method of editing a nucleic acid, said repair template comprising an end which matches a cut end of a cut (genomic) nucleic acid to be edited and a sequence which is homologous to a sequence of the (genomic) nucleic acid to be edited.

The step of cutting the nucleic acid to be edited may use a nuclease which creates staggered cuts, especially staggered cuts in dsDNA. It should be noted that a staggered cut may comprise a break (in the nucleic acid to be edited) characterized by overhanging sequences. These over hangs may be 5′ overhangs. Cuts of this type may be commonly referred to as ‘sticky-end’ or ‘sticky-ended’ cuts. Whatever nuclease is used to cut the nucleic acid, that nuclease may yield a staggered (sticky-end) cut having a set number of nucleotides with the overhang sequence. By way of example a sticky-ended cut may comprise a 5′-overhang, with 2, 3, 4, 5, 6 or 7 nucleotides. In one teaching, a 5′ overhang (sticky-ended cut) may comprise 4 or 5 nucleotides.

The step of cutting the nucleic acid to be edited may use a nuclease of the CRISPR-Cas system.

The step of cutting the nucleic acid may use a Cas12a nuclease. As such, a method of this disclosure may comprise:
cutting the nucleic acid sequence to be edited with Cas12a; and
contacting the cut nucleic acid sequence with a nucleic acid repair template;
wherein the nucleic acid repair template comprises an end which matches a cut end of the nucleic acid sequence to be edited and a sequence which is homologous to a sequence of the nucleic acid sequence to be edited.

Cas12a (previously known as Cpf1) is a single-effector CRISPR protein. Unlike Cas9, Cas12a naturally employs a single CRISPR RNA (crRNA) as guide RNA, which is substantially shorter than the engineered single guide RNA (sgRNA) used for Cas9. Moreover, Cas12a recognizes a T-rich protospacer adjacent motif (PAM) sequence, comparing to the G-rich PAM recognized by Cas9. Cas12a uses a single RuvC catalytic domain to cleave both the target and non-target strands, this creates 5′-overhang sticky ends (in contrast, Cas9 employs two nuclease domains RuvC and HNH generating a blunt-ended double-stranded break at the target locus).

The term Cas12a (as used herein), embraces any of the recognized Cas12a homologs, including, for example FnCas12a (from Francisella novicida), LbCas12a (from Lachnospiraceae bacterium) and AsCas12a (from Acidaminococcus sp.). The term ‘Cas12a’ may also embrace any functional form of Cas12a including, for example, any fragments which retain an ability to cut (or cleave) a nucleic acid to create 5′-overhang sticky ends. In one teaching the Cas12 nuclease is AsCas12a.

In use, a Cas12a nuclease will cut both strands of the nucleic acid to be edited (i.e. both the target and the non-target strand) to yield two nucleic acid fragments, both having 5′-overhang sequences (so called 5′ overhang sticky ends).

A method of this disclosure may use a single Cas12a molecule in order to cut the nucleic acid sequence creating a single double stranded break in the nucleic acid (i.e. the nucleic acid to be edited).

The nucleic acid to be edited may comprise a protospacer adjacent motif (PAM) or PAM sequence. The site at which the nuclease (for example a Cas12a nuclease) cuts, may lie distal to the PAM site. Where the nuclease (for cutting the nucleic acid sequence to be edited) is a Cas12a nuclease, the PAM sequence is a T-rich PAM sequence.

The nucleic acid to be edited may comprise a Cas12a cleavage site.

The nucleic acid to be edited may comprise a guide RNA or CRISPR RNA binding site. Further detail regarding any gRNA and/or crRNA component is provided below.

The target region of the nucleic acid sequence to be edited may lie adjacent (or downstream of) the Cas12a cleavage site. The specific sequence to be altered may lie within a few, for example 1, 2, 3, 4, 5, 10, 15, 20 or more, base pairs of the cleavage site.

A repair template for use in a method of this disclosure may comprise a double stranded nucleic acid sequence. The repair template may comprise a single stranded sequence. The repair template may be a fully or partial double-stranded repair template.

As stated, the repair template comprises an end which matches a cut end of the nucleic acid to be edited and a sequence which is homologous to a part of that nucleic acid.

The end of the repair template which matches a cut end of the nucleic acid to be edited, may be referred to as a ‘cut-end matching overhang sequence’. The cut-end matching overhang sequence may comprise a 5′-overhang sequence (namely a ‘cut end matching 5′-overhang sequence’). The cut-end matching 5′-overhang sequence may match the sequence of the 5′-overhang of one of the nuclease cut ends of the nucleic acid to be edited.

One of skill will appreciate that when a nucleic acid sequence is cut (or cleaved) using a nuclease as described herein (for example Cas12a), two staggered and ‘sticky’ cut ends are generated. Each staggered/sticky end comprises a 5′-overhang sequence. Cutting a nucleic acid sequence as described herein (for example with Cas12a) will generate two cut ends a left-hand, or proximal, cut end and a right-hand, or distal, cut end.

The cut end matching 5′-overhang sequence a repair template for use in a method of this disclosure may match the sequence of a distal 5′-overhang cut end. For example, the cut matching 5′ overhang sequence of a repair template for use in a method of this disclosure may comprise a sequence which matches the sequence of a distal 5′-overhang cut end generated by Cas12a.

The other end of the repair template may also be sticky-ended (i.e. comprise a sequence overhang (e.g. a 5′-overhang)).

Alternatively, the other end may be blunt-ended.

Accordingly a repair template for use in a method of this disclosure may comprise two ends, at least one of which is ‘sticky-end’ with a 5′-overhang sequence, the sequence of which matches the sequence of one of the cut ends of the nucleic acid sequence to be edited and one other sticky end or a blunt end. Where the nucleic acid to be edited is cut using Cas12a, the repair template may comprise at least one end in which the sequence of the 5′-overhang matches the sequence of one of the cut ends generated by the Cas12a nuclease.

The cut end matching 5′-overhang sequence of the repair template may comprise the same or a different (for example a lesser) number of nucleotides as present in the 5′ overhang of the (distal) cut end of the nucleic acid sequence to be edited. Where the repair template comprises fewer nucleotides in its cut-end matching 5′ overhang, those nucleic acids will match (i.e. correspond to) at least some of the nucleotides present in sequence of the (distal) cut-end 5′-overhang.

The cut end matching 5′-overhang sequence (of the repair template) may comprise 2 nucleotides, 3 nucleotides, 4 nucleotides or 5 nucleotides.

In one teaching, the cut end matching 5′-overhang sequence (of the repair template) may comprise 4 nucleotides. The 5′-overhang sequence of the (distal) cut end of the nucleic acid sequence to be edited may also comprise 4 nucleotides. Those 4 nucleotides may match or be the same as 4 of the nucleotides present in the cut matching 5′ overhang sequence of the repair template.

In one teaching, the cut end matching 5′-overhang sequence (of the repair template) may comprise 5 nucleotides. The 5′-overhang sequence of the (distal) cut end of the nucleic acid sequence to be edited may also comprise 5 nucleotides. Those 5 nucleotides may match or be the same as 5 of the nucleotides present in the cut matching 5′ overhang sequence of the repair template.

The part of the repair template which comprises a sequence which is homologous to part of the nucleic acid to be edited, may be homologous to all or part of the target region of that nucleic acid sequence. For convenience, this sequence shall be referred to as the ‘homologous sequence’.

The homologous sequence may comprise the intended sequence edit—that is the sequence that is to replace or modify a sequence of the target region or the sequence which introduces an additional nucleic acid sequence into the target region.

In one teaching, the homologous sequence (of the repair template) may be substantially identical to all or part of the target region.

As stated, the homologous sequence of the repair template may be fully or partially double stranded. For example, the homologous sequence may comprise both double stranded parts and single stranded parts.

Relative to the sequence of the target region, the homologous sequence may comprise one or more nucleobase alterations—for example the inclusion of additional nucleobases (not present in the sequence of the target region) and/or the omission of other nucleobases which are present in the target region. Where the target region comprises a mutation (for example a deleterious mutation, the homologous sequence of the repair template may comprise a correction—i.e. the correct nucleotide base pairs.

The homologous sequence may comprise a sequence which differs from the sequence of all or part of the target region by the presence of:
(i) one or more additional nucleobase(s); and/or
(ii) the absence of one or more one or more nucleobases present in the target region.

The homologous sequence may comprise between about 1 and about 300 bases and/or base pairs.

The homologous sequence may comprise between about 1, about 2, about 3, about 4, about 5, about 6, about 7, about 8, about 9 or about 10 and about 250 bases and/or base pairs.

The homologous sequence may comprise between about 20 and about 200 base pairs.

The homologous sequence may comprise about 25, about 30, about 35, about 40, about 45, about 50 about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, or about 195 bases and/or base pairs.

The homologous sequence may comprise about 40 to about 120 bases and/or base pairs.

The homologous sequence may comprise about 60 to about 100 bases and/or base pairs.

The homologous sequence may comprise 80 bases and/or base pairs.

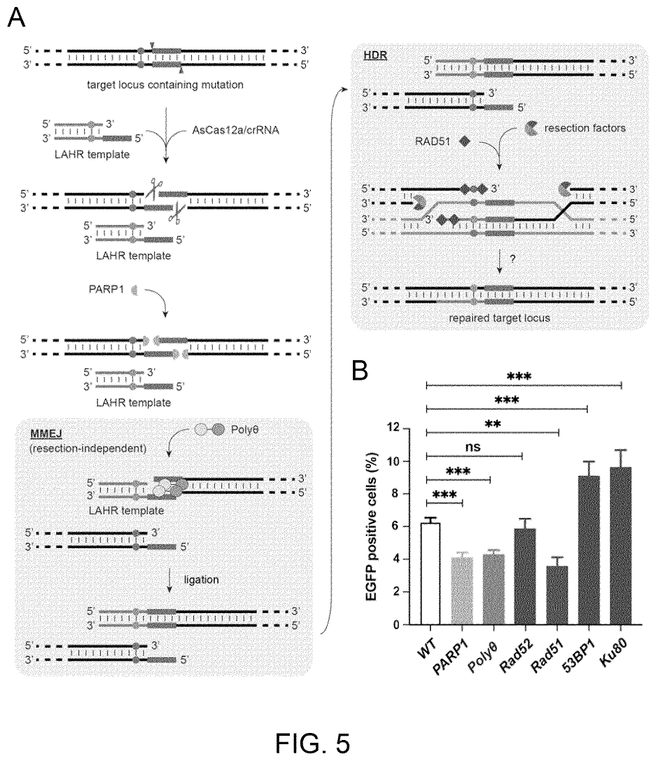

The homologous sequence may comprise, at least a single strand of nucleic acid which is homologous to sequence of the nucleic acid to be edited. All or part of that homologous sequence may further comprise an additional nucleic acid strand forming base pairs with some or all of the bases of the single strand. For example, the homologous sequence may comprise, for example 1-300, for example 20, 40, 60, 80, 100 or 120 bases (which bases are homologous to a sequence of the nucleic acid to be edited). At least some of those bases may be paired to other bases to form a double stranded homologous sequence. For example, 1-60 (for example 2, 4, 6, 8, 10, 15, 20, 25, 30, 35, 40, 45, 50 or 55) bases of the single strand of the homologous sequence may be pair to other bases to form a (partial) double strand). FIG. 5 presented here in shows repair templates having partial and/or complete double stranded homologous sequences.

One of skill will appreciate that the exact size (in terms of number of bases and/or base pairs) of the homologous sequence may vary. The variance may depend on the size of the target region within the nucleic acid sequence to be edited (longer target regions may require longer homologous sequences) and or the number of edits that need to be made to the target region.

As stated, the homologous sequence may match at least part of the sequence of the target region.

The abovementioned homologous sequence may be disposed (in the repair template) between the blunt end and the sticky end.

In view of the above, the repair template may comprise a double stranded nucleic acid sequence comprising a short dsDNA homologous arm (comprising the homologous sequence) and a “sticky” 5′-overhang end which matches an AsCas12a-generated cut end.

The repair template (and methods using the same) functions to deliver a desired sequence to a target region within a nucleic acid sequence to be edited. For example, the repair template may deliver:
a nucleotide substitution; and/or
a nucleotide addition; and/or
a nucleotide deletion;
   to a target region within a nucleic acid sequence to be edited which substitution. In such cases, the desired sequence and/or any of the nucleotide substitutions(s), addition(s) and/or deletion(s) may be located or included within the homologous sequence (or arm) of the repair template.

In one teaching, a method of editing a nucleic acid sequence may comprise:
providing or obtaining a nucleic acid sequence to be edited,
cutting or cleaving the nucleic acid sequence to be edited with a Cas12a nuclease to yield two fragments comprising proximal and distal cut ends; and
contacting the cut nucleic acid sequence with a nucleic acid repair template; wherein the nucleic acid repair template comprises:
an end which matches the distal Cas12a cut end of one of the nucleic acid fragments; and
a sequence which is homologous to a target region of the nucleic acid sequence and which contains the sequence edit.

In all the disclosed methods the step of contacting may take place under conditions which facilitate editing of the nucleic acid sequence by replacement of a sequence within a target region of the nucleic acid to be edited with a nucleic acid sequence comprised within the repair template (specifically, for example, the nucleic acid sequence provided by the homologous sequence of the repair template). Those conditions may facilitate editing via both homology directed repair (HDR) and microhomology-mediated end joining (MMEJ) mechanisms. This combined approach is advantageous and the inventors have adopted the term ‘Ligation-Assisted Homologous Recombination’ (LAHR) to describe the various novel (HDR/MMEJ) methods described herein.

As compared to prior art methods, the LAHR methods described herein achieve relatively high levels of editing efficiency. Accordingly, the methods of this disclosure may be used to efficiently:
repair nucleic acid sequences/genes;
rescue sequence/genes; and/or
correct sequences/genes.

Further, as compared to prior art methods, the LAHR methods described herein can achieve high levels of editing efficiency without the use of one or more DNA repair inhibitors, such as BAY-598 and/or NU7441. Other DNA repair inhibitors may include SB939, A196, KY02111, R-PFI-2-hydrochloride, A395 and/or AT9283. Moreover, in the disclosed methods the AsCas12a-cleaved genomic DSB end and repair template may both contain homologous 5′ overhangs, such that they enter the MMEJ pathway at the level of Polyθ, skipping the need of strand resection. The remaining homologous arm of the template recombines by HDR. As a consequence the disclosed methods provide a novel method capable of precisely editing the genome, which (as stated) does not require the use of DNA repair inhibitors.

The methods of this disclosure may further comprise the use of a guide RNA (gRNA) most commonly known as a CRISPR RNA (crRNA). Where the nuclease is a Cas12a nuclease, the method may use is single crRNA. The role of the crRNA is to guide the nuclease to the cleavage site. The crRNA may be synthetic and one of skill will know that the sequence of any necessary crRNA may vary depending on the sequence of the nucleic acid to be edited.

This disclosure further provides a kit for repairing a nucleic acid sequence, the kit comprising a repair template as described herein.

A kit of this disclosure may further comprise:
instructions for use; and/or
a nuclease (for example a Cas12a nuclease) for cleaving a nucleic acid sequence to be edited; and/or
a guide RNA (gRNA) or CRISPR RNA (crRNA); and/or
receptacles, tool and/or buffers.

### DETAILED DESCRIPTION

The present invention will be described by reference to the following figures which show:

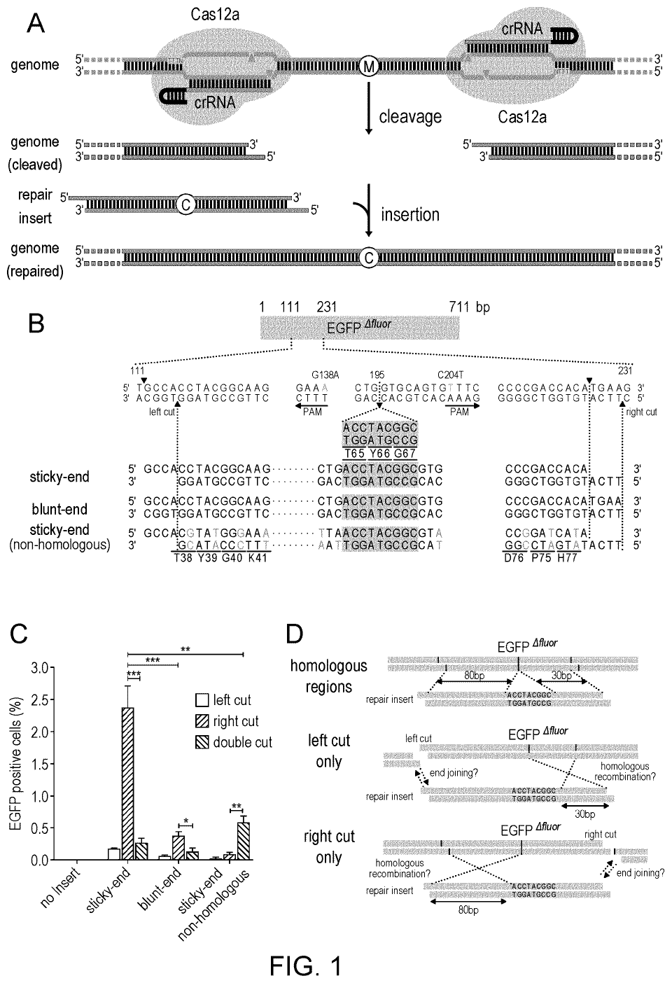

FIG. 1: Conducting precise genome editing by CAPR. (A) A schematic representation of the CAPR strategy, in which two molecules of Cas12a protein were guided by two crRNAs to target and excise a genomic region containing a malicious mutation (red-circled M); the cleavage created two staggered ends; a given repair insert possessing two compatible sticky ends and a correction (blue-circled C) ligated to the compatible ends on the genome to perform the repair. (B) A schematic representation of the EGFP^{Δfluor} reporter. The AsCas12a editing regions between T111 and G231 were shown in a double-stranded format, in which silent mutations G138A and C204T (red uppercases) were introduced to generate two AsCas12a PAM sites (underlined by blue arrows, and the arrows orientate the direction of the PAM sequences). Two AsCas12a cut sites: ‘left cut’ and ‘right cut’ were indicated by red arrowheads. The fluorophore-coding sequence (encoding T65, Y66 and G67) removed behind G195 was green-highlighted. Three repair inserts, ‘sticky-end’, ‘blunt-ended’ and ‘sticky-ended non-homologous’ carried the fluorophore-coding sequence (green-highlighted). The ‘sticky-ended non-homologous’ insert contained multiple silent mutations (red uppercases) to demolish the homology, and the corresponding amino acids were underneath. (C) The EGFP^{Δfluor} mutant was repaired by different repair inserts in different cleavage scenarios. The repair efficiency was indicated by the percentage of EGFP positive cells from FACS analysis. Error bars corresponded to the standard deviation of the average of n=3 parallel samples. The experiment was repeated three times and a representative dataset was presented here. Statistical test: two-tailed unpaired t-test, * P<0.05, ** P<0.01, *** P<0.001. (D) A hypothetic model of the repair in the context of the Cas12a cut. The repair insert contained two homologous regions to the EGFP^{Δflour} reporter gene, 80 bp and 30 bp respectively, flanking the EGFP fluorophore-coding sequence (green highlighted). In the circumstance of ‘left cut only’, the effective homologous arm of the repair insert was 30 bp, while the cut only at the right side, the effective homologous arm was 80 bp.

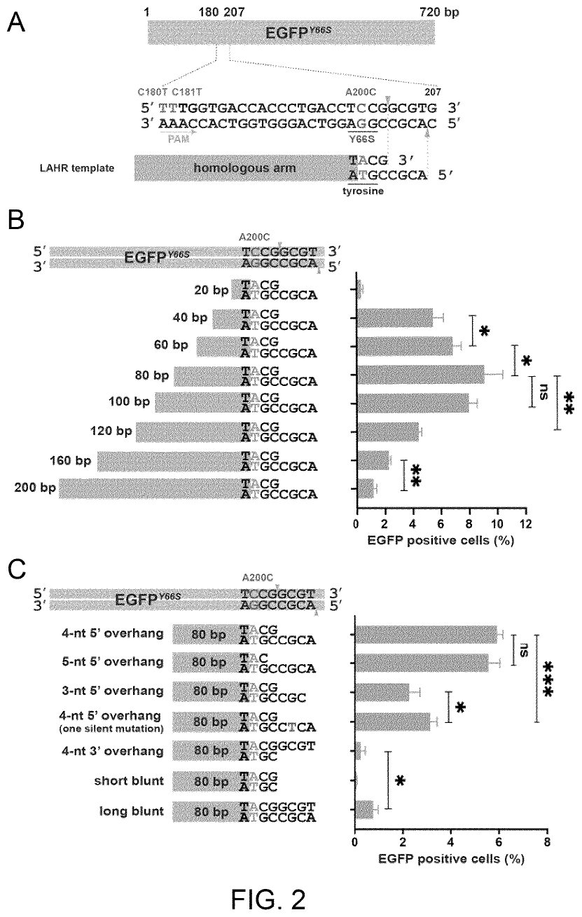

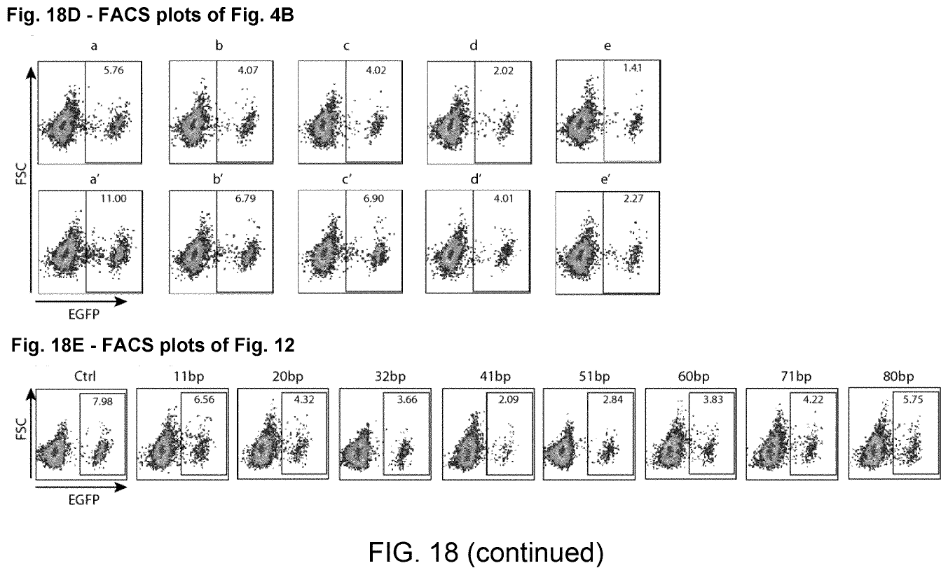

FIG. 2: LAHR utilizes both the homologous arm and the sticky end of the repair template (A) A schematic of the EGFP^{Y66S} reporter sequence and the repair template. The dsDNA region between T180 and G207 indicated the AsCas12a editing region, in which the silent mutations, C180T and C181T (blue uppercases) were introduced to generate an AsCas12 PAM sequence (underlined by a green arrow, and the arrow orientated the direction of the PAM sequence); the cleavage site was indicated by green arrowheads; the missense mutation A200C (red uppercase) caused a tyrosine-to-serine substitution (Y66S) that eliminated the EGFP fluorescence. The repair template contained a sticky end, which was compatible to the AsCas12a-generated distal sticky end on the reporter gene, and a homologous arm (green box). Adjacent to the sticky end of the repair template a repairing A/T base pair (green uppercases) was introduced to restore the codon of tyrosine. (B) The correction of the A200C mutation using repair templates with a same sticky end, but varying lengths of the homologous arms (from 20 bp to 200 bp). The graph depicted the percentage of EGFP-positive cells from the FACS analysis (FIG. 18). Error bars corresponded to the standard deviation of the average of n=3 parallel samples. The experiment was repeated three times and a representative dataset was presented here. Statistical test: two-tailed unpaired t-test, ns P>0.05, * P<0.05, ** P<0.01. (C) The same correction conducted with the repair templates sharing a same 80-bp homologous arm, but with indicated sticky ends. The repair efficiency was presented in the percentage of EGFP positive cells from FACS analysis (FIG. 18). Error bars corresponded to the standard deviation of the average of n=3 parallel samples. The experiment was repeated three times and a representative dataset was presented here. Statistical test: two-tailed unpaired t-test, ns P>0.05, * P<0.05, *** P<0.001.

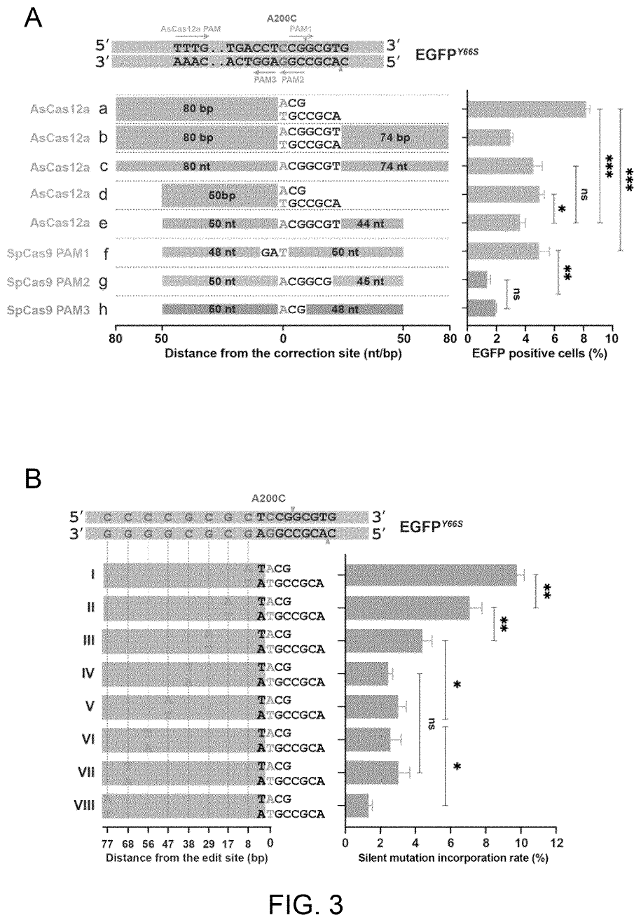

FIG. 3: Characterization of LAHR. (A) Comparison between LAHR and HDR in repair of the EGFPY66S reporter. Cells were targeted using AsCas12a RNP and different repair templates as indicated, (a) a LAHR template with an 80-bp homologous arm and a compatible sticky end; (b) a 160-bp dsDNA template; (c) a 160-nt ssODN template; (d) a LAHR template with a 50-bp homologous arm; (e) a 100-nt ssODN template. For the SpCas9 targeting, the following repair templates were used: (f) a 100-nt ssODN template for PAM1; (g) a 100-nt ssODN template for PAM2; (h) a 100-nt ssODN template for PAM3. Templates (g) and (h) were a same sequence, and the template (f) was the reverse complement sequence of it. In all repair templates from (a) to (f), homologous arms of each template were presented as coloured boxes, and different colours indicated different PAM usages. The numbers in the boxes indicated the size of the homologous arm. The correction base (A or T) or base pair (A/T) was in green uppercases. The scale beneath repair templates indicated the distance between each end and the correction site. Data were shown as the percentage of EGFP positive cells from FACS analysis (FIG. 18). Error bars corresponded to the standard deviation of the average of n=3 parallel samples. The experiment was repeated three times and a representative dataset was presented here. Statistical test: two-tailed unpaired t-test, ns P>0.05, * P<0.05, ** P<0.01, *** P<0.001. (B) The repair templates (I-VIII) contained the same A/T base pair (green uppercase) to correct the A200C mutation. A base pair (blue uppercases) to introduce silent mutation on each template was distributed along the homologous arm (green box). The scale at bottom indicated the distance between the silent-mutation-inducing substitution and the repairing A/T base pair. Data were shown as the percentage of each silent mutation incorporation rate based a NGS analysis. Error bars corresponded to the standard deviation of the average of n=3 parallel samples. Statistical test: two-tailed unpaired t-test, ns P>0.05, * P<0.05.

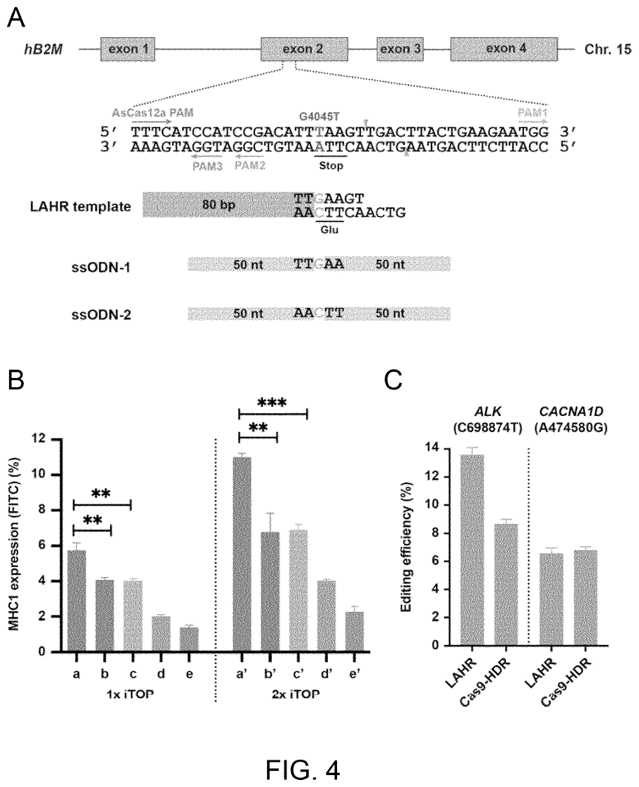

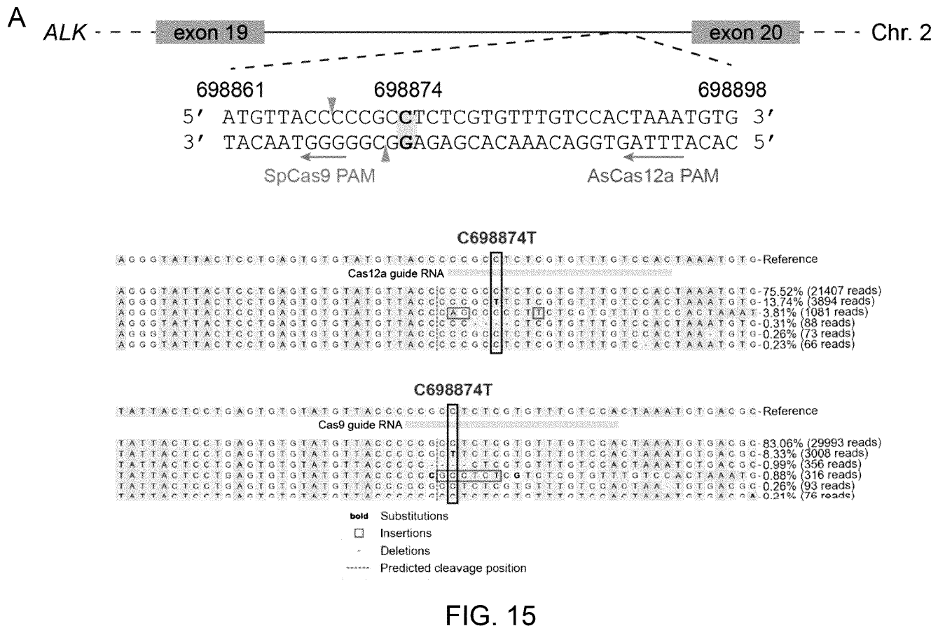

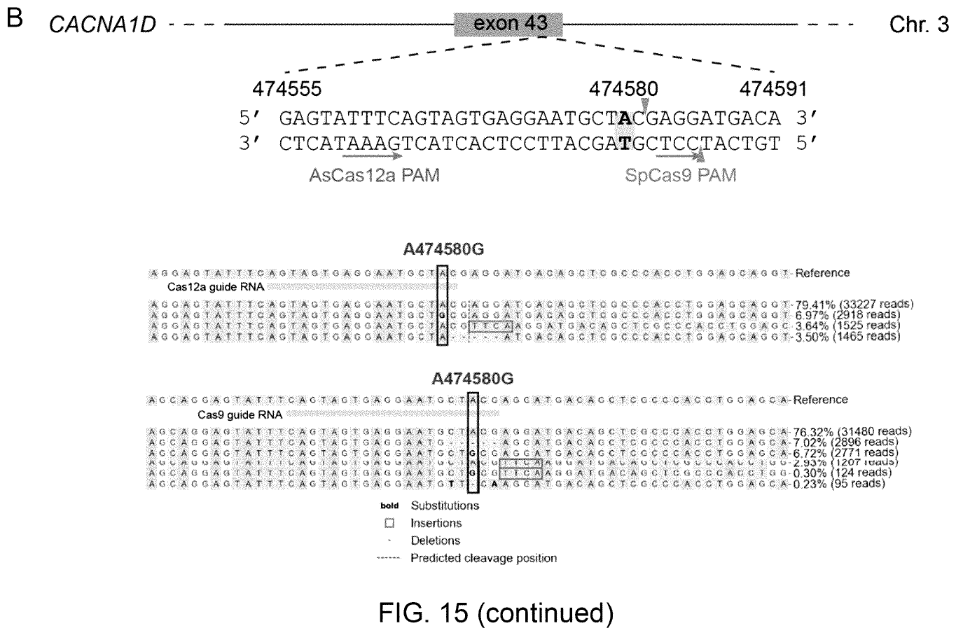

FIG. 4: Introducing single nucleotide substitutions in endogenous genes by LAHR or HDR (A) The nonsense mutation G4045T (in red text) that introduced a premature stop codon (underlined) was located on the exon 2 of the B2M gene in HAP1B2M−/− cells. Around the mutation locus, there were one AsCas12 PAM (green arrow) and three SpCas9 PAMs (orange, grey and blue arrows) which could be used to induce DSBs. The AsCas12a cleavage site was indicated by green arrowheads. Besides the LAHR template, two 100-nt ssODN templates, ssODN-1 (from the positive strand) and ssODN-2 (from the negative strand) were used to repair the DSBs induced by AsCas12a or SpCas9 using different PAMs. (B) The bar chart showed the repair efficiencies of different experimental setups, which were indicated by the percentage of FITC positive cells from FACS analysis. The colours of columns which indicated different PAM usage were consistent with the colours of PAMs shown in (A). Column a and a′ indicated the LAHR repair; b and b′ indicated the AsCas12a-mediated HDR using the template ssODN-1; c and c′ indicated the SpCas9-induced DSB (PAM1) using the template ssODN-2; d and d′ indicated the SpCas9-induced DSB (PAM2) using the template ssODN-1; e and e′ indicated the SpCas9-induced DSB (PAM3) using the template ssODN-1. For the column a-e, one-round iTOP transduction was performed, while the column a′-e′ exhibited the repair efficiencies from two rounds of iTOP transductions. Data were shown as the percentage of EGFP positive cells from FACS analysis (FIG. 18). Error bars corresponded to the standard deviation of the average of n=3 parallel samples. The experiment was repeated three times and a representative dataset was presented here. Statistical test: two-tailed unpaired t-test, ** P<0.01, *** P<0.001. (C) Two single nucleotide substitutions, C698874T and A474580G, were respectively introduced in human ALK and CACNA1 D (FIG. 15) by LAHR and Cas9-mediated HDR. The bar chart showed the comparison of the editing efficiencies between LAHR and Cas9-mediated HDR. Error bars corresponded to the standard deviation of the average of n=3 independent biological replicates.

FIG. 5: DNA repair mechanisms underlying LAHR. (A) A hypothetic model of LAHR. AsCas12a/crRNA RNP cleaved a target sequence containing a mutation (red dots on both strands) at an AsCas12a cut site (indicated by purple arrowheads on both strands). The AsCas12a-induced DSB yielded two staggered ends possessing 5′ homologous overhangs (blue blocks). After DSB occurred, PARP1 detected and accumulated at the DSB ends to recruit downstream factors involved in different DNA DSB repair pathways. The following MMEJ process was highlighted in a light green box, where the recruited Polyθ paired two compatible 5′ homologous overhangs located on the downstream DSB end and on the LAHR template respectively. As the MMEJ process here did not involve resection, thus it was marked as ‘resection-independent’. After the LAHR template ligated to the downstream DSB end by the resection-independent MMEJ, HDR (highlighted in a light blue box) took place to incorporate the correct base (green dots on the LAHR template) into genome. The orange dsDNA template indicated a sister chromatid. The question mark at the last step of HDR indicated a potential mismatch repair procedure involved to convert the mutation to a correct base. (B) To test the hypothesis, selected gene targets were knocked down by siRNA targeting. The bar graph showed the repair efficiencies of EGFPY66S cells under different gene knockdown conditions, which were indicated by the percentages of EGFP positive cells from FACS analysis (FIG. 18). Error bars corresponded to the standard deviation of the average of n=3 parallel samples. The experiment was repeated three times and a representative dataset was presented here. Statistical test: two-tailed unpaired t-test, ns P>0.05, ** P<0.01, *** P<0.001.

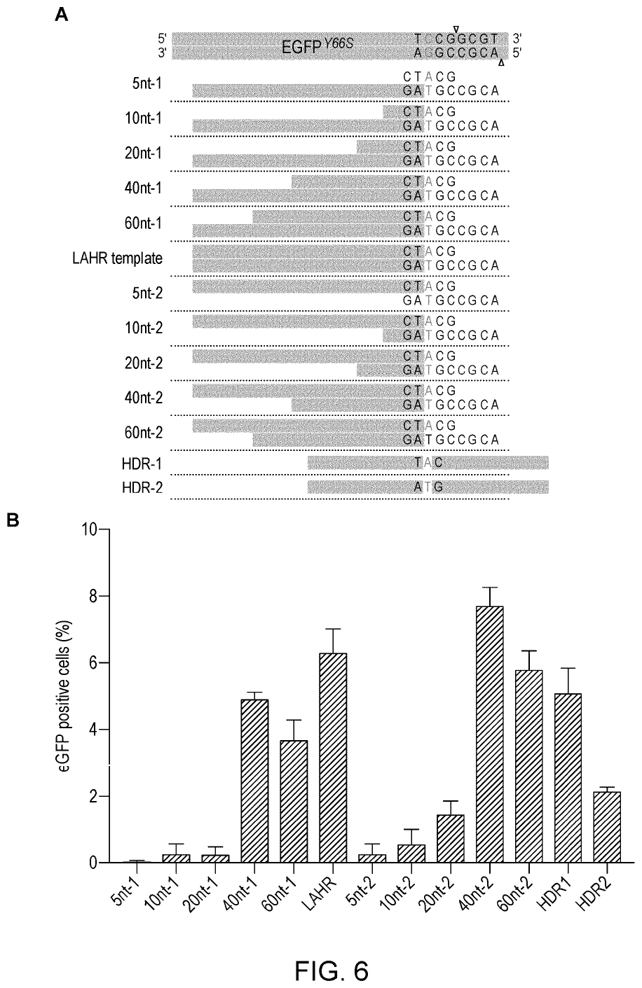

FIG. 6: Partially double-stranded template. A: A schematic of the EGFPY66S reporter sequence and the used repair templates containing a matching 5′-overhang sticky end and either fully double-stranded homologous sequence (LAHR template) or different lengths of the upper or lower strand as indicated. As controls, single-stranded oligonucleotide repair templates (ssODN) of only the top or the bottom strand were used (HDR-1 and HDR-2). B: Editing efficiency using the different repair templates depicted in (A). EGFPY66S reporter cells were transduced with recombinant Casa12a protein, crRNA targeting the EGFPY66S site (nuclease cut sites indicated with green arrows in A) and the indicated templates using iTOP transduction. Three days after transduction, % eGFP expression was used to measure editing efficiency by flow cytometry.

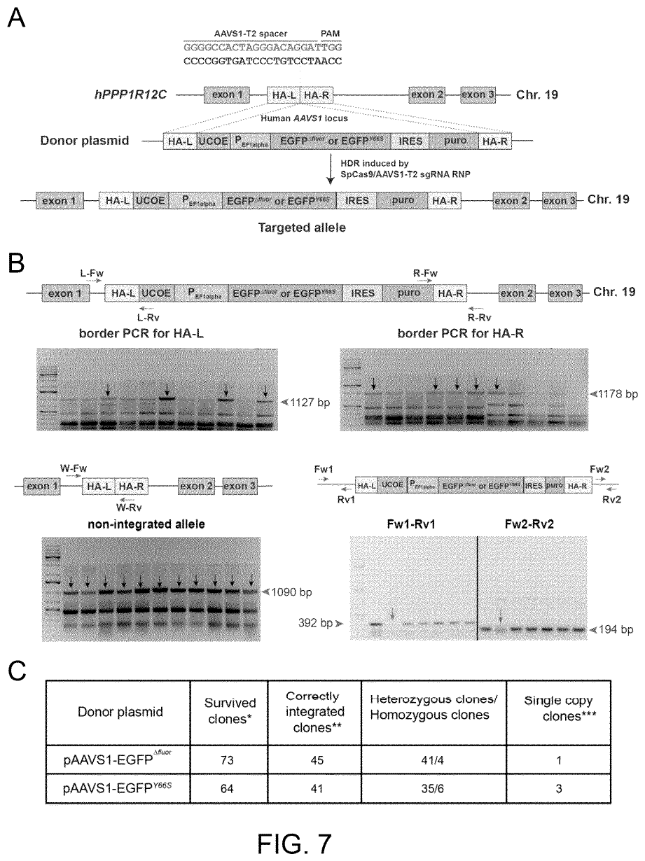

FIG. 7: Building the single-copy EGFP^{Δfluor}/EGFP^{Y66S} reporter HAP1 cell lines. (A) The scheme showed the human AAVS1 locus where we cloned two homologous arms (HA-L and HA-R) to build the donor plasmids. The AAVS1-T2 spacer spanned the border between HA-L and HA-R regions on the genome. The corresponding single guide RNA (sgRNA) targeting the AAVS1-T2 spacer sequence together with SpCas9 protein and donor plasmid were electroporated into HAP1 cells. The SpCas9-induced DNA DSB facilitated the HDR using the donor plasmid as repair template. The correctly targeted allele contained a UCOE (Ubiquitous Chromatin Opening Element) sequence, a human EF1alpha promoter, an EGFP^{Δfluor} or EGFP^{Y66S} reporter gene, an IRES (Internal Ribosome Entry Site) sequence and a puromycin resistant gene, which were flanked by two homologous arms. (B) To verify the correct single allele targeting, we utilized a PCR based strategy. Firstly, the PCR product across the HA-L border (using the primer pair L-Fw/L-Rv) verified the correct homologous recombination of HA-L, which was indicated by a 1127-bp band on the gel image (black arrows represented the positive integrations). The HA-L-integrated clones were selected to proceed with the HA-R border PCR analysis using the primer pair R-Fw/R-Rv, and a 1178-bp band indicated a correct integration of HA-R. Subsequently, the double positive clones from the border PCR analysis were further screened by another round of PCR using the primer set W-Fw and W-Rv, which selected the clones containing a non-integrated allele (a 1090-bp band on the gel image). Finally, two primer sets, Fw1/Rv1 and Fw2/Rv2, targeting two small regions located on the donor plasmid outside the ‘HA-L-reporter-HA-R’ region were used to exclude the random integration of the donor plasmid. The 392-bp and 194-bp bands indicated the corresponding plasmid regions were randomly integrated in the genome, whereas the double negative clones (red arrows) from this analysis were finally selected as the correct single-copy reporter clones. The gel images shown here were selected to represent the principle. (C) Clone screening. * Survived clones from a full 96-well plate of single cells from single-cell sorting, ** Double positive clones from a border PCR screen, *** Single-copy reporter clones from a PCR-based zygosity screen. PCR primers are listed in Table 1.

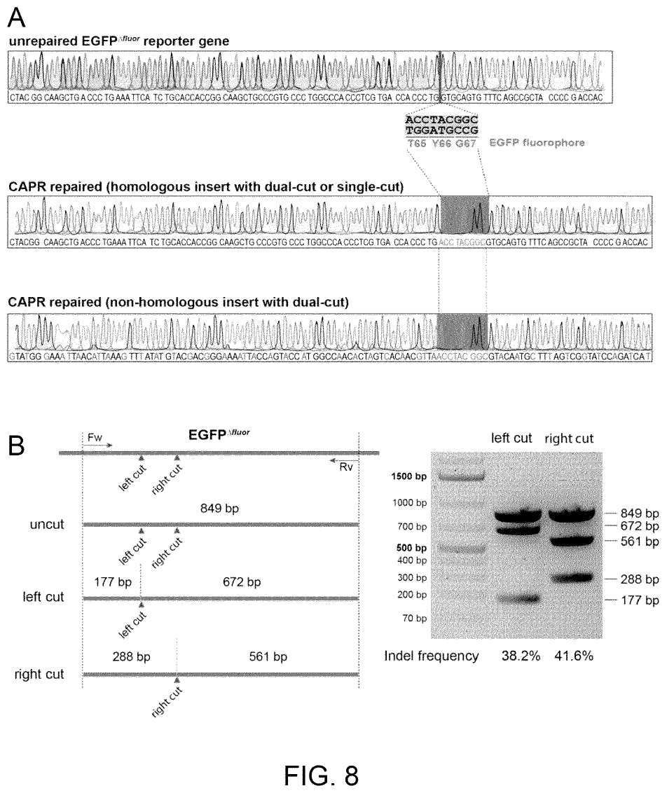

FIG. 8: (A) Verifying CAPR by Sanger sequencing. To confirm the repair of EGFP^{Δfluor} reporter cells by CAPR (including ‘single-cut’ controls), EGFP positive cells were FACS-sorted in both bulk and single cells. For each edited sample, the genomic DNA was isolated from both bulk EGFP positive cells and 20 EGFP positive clones respectively. The target locus on the genomic DNA then was amplified by PCR, and the PCR products were further analyzed by Sanger sequencing. Chromatograms, from top to bottom, exhibited unrepaired fluorophore-removal locus of the EGFP^{Δfluor} reporter gene, the EGFP fluorophore restored by the homologous repair insert with dual or single AsCas12a cleavage and the repair using a non-homologous repair insert. In the chromatograms showing the repaired EGFP^{Δfluor} reporter genes, the recovered EGFP fluorophore coding region was covered by green shadow. Because all the sequencing data did not show mutations, we only present one chromatogram from each group. (B) Indel efficiencies of AsCas12a-created ‘left cut’ and ‘right cut’ on the EGFP^{Δfluor} reporter gene. To assess the indel efficiencies of two AsCas12a cleavage sites, ‘left cut’ and ‘right cut’, on the EGFP^{Δfluor} reporter gene (we used these two sites in CAPR studies. FIG. 1). AsCas12a protein and crRNA targeting each site were respectively transduced into the single-copy EGFP^{Δfluor} reporter cells. Following the genomic DNA isolation, the PCR amplified targeting locus was used to perform T7E1 assay. The schematic showed the cleavage patterns of uncut, left-cut and right-cut PCR products after T7 endonuclease treatment. The agarose gel showed T7 endonuclease-resulting fragments. Band sizes were indicated at the right side of the gel image. The indel efficiencies were quantified by ImageJ based on the gel image.

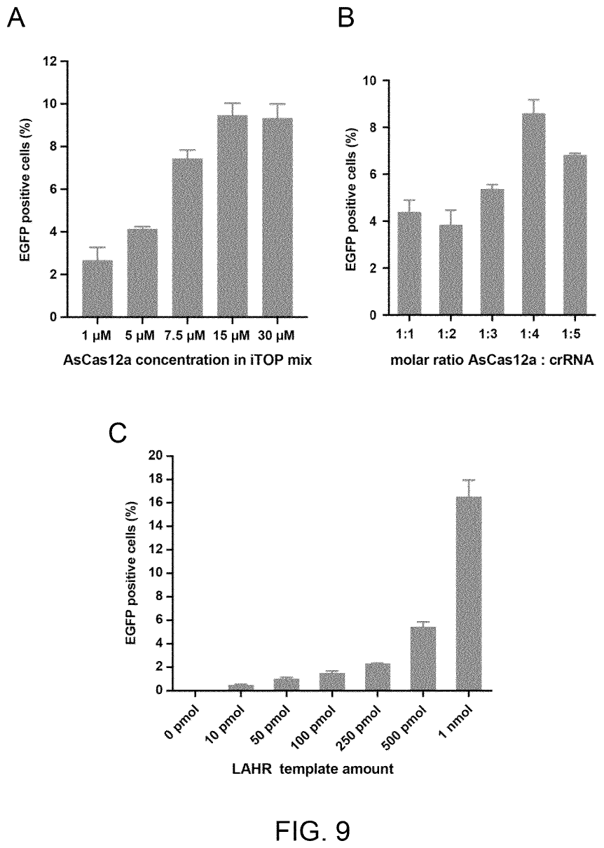

FIG. 9: Optimizing quantities and ratios of LAHR components. (A) To investigate how the quantity of AsCas12a protein influenced the LAHR efficiency, we tested a AsCas12a protein gradient in repair of the EGFP^{Y66S} mutant in the reporter cells. When the final concentration of AsCas12a reached 15 μM, the LAHR efficiency got plateaued. Error bars corresponded to the standard deviation of the average of n=3 parallel samples. The experiment was repeated three times and a representative dataset was presented here. (B) With different molar ratios between AsCas12 protein and crRNA, LAHR were performed to repair the EGFP^{Y66S} mutant. The experiment showed that 1:4 is the optimized ratio. Error bars corresponded to the standard deviation of the average of n=3 parallel samples. The experiment was repeated three times and a representative dataset was presented here. (C) The repair efficiency showed a positive correlation with the increasing amount of LAHR template. Error bars corresponded to the standard deviation of the average of n=3 parallel samples. The experiment was repeated three times and a representative dataset was presented here.

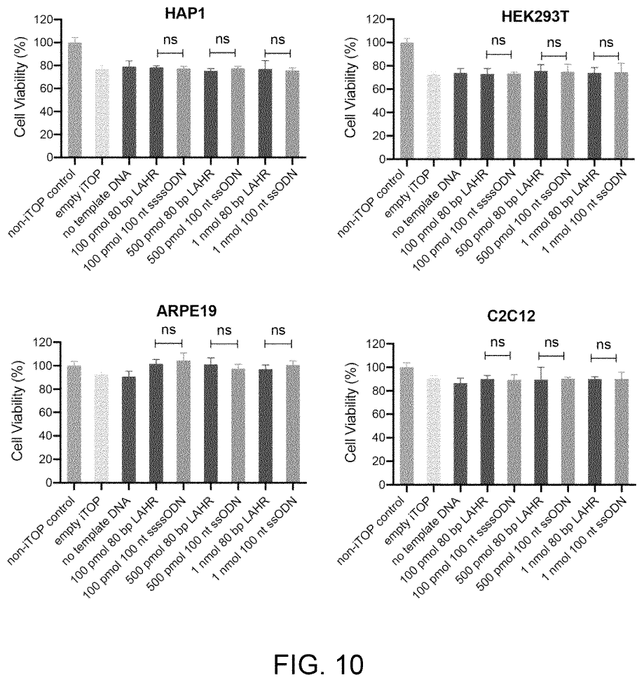

FIG. 10: Cell viability assessment following iTOP transduction of LAHR components. The post-iTOP cell viabilities of different cell lines were assessed by MTS assay. The MTS assay was performed 24 hours after iTOP transduction. The bar graphs showed the viabilities of different cell lines that performed iTOP deliveries of 80-bp LAHR or 100-nt ssODN template in different quantities, together with AsCas12a protein and crRNA. We observed no significant difference in cell viability between the empty iTOP and the no-template DNA controls and any of the tested template samples. Error bars corresponded to the standard deviation of the average of n=3 parallel samples. The experiment was repeated three times and a representative dataset was presented here. Statistical test: two-tailed unpaired t-test, ns P>0.05.

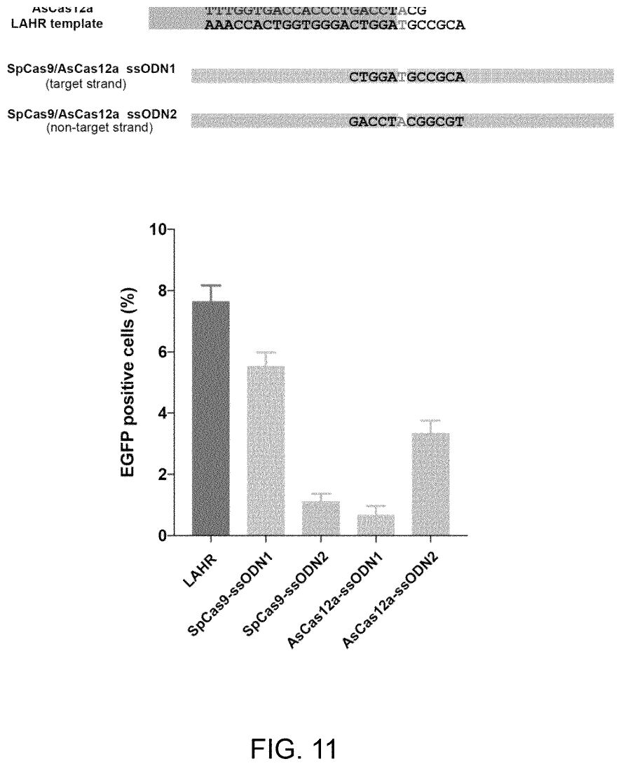

FIG. 11: Supplementary comparisons for FIG. 3A. Here, we compared the LAHR to HDRs using reverse complementary ssODN templates (ssODN1 and ssODN2). Since the AsCas12a PAM (green arrow) and SpCas9 PAM (orange arrow) had the same orientation, for both, the top strand was a ‘non-target strand’ and the bottom strand was a ‘target strand’. The ssODN1 from the ‘target strand’ favored the Cas9-mediated HDR, while the ssODN2 from the ‘non-target strand’ favored Cas12a-mediated HDR. This result was consistent with the previously published data (1,2). Error bars corresponded to the standard deviation of the average of n=3 parallel samples. The experiment was repeated three times and a representative dataset was presented here.

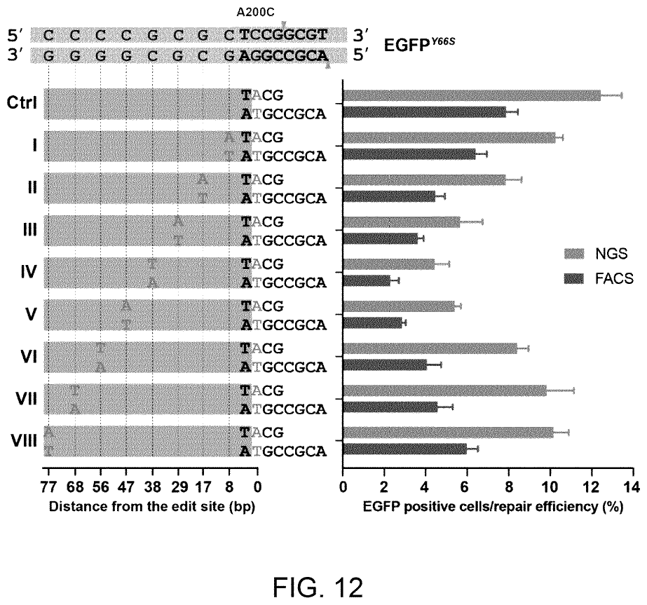

FIG. 12: FACS and NGS analyses for the LAHR-mediated repair on the ‘A200C’ mutation. The ‘A200C’ mutation that turned EGFP off in the EGFP^{Y66S} reporter cells was indicated as a red C/G pair. LAHR templates I to VIII contained intended substitutions (blue base pairs) to introduce silent mutations. FACS data (Green bars) showed the percentages of EGFP positive cells after LAHR. The gray bars indicated the actual repair efficiency of the A200C mutation using different templates. Error bars corresponded to the standard deviation of the average of n=3 parallel samples.

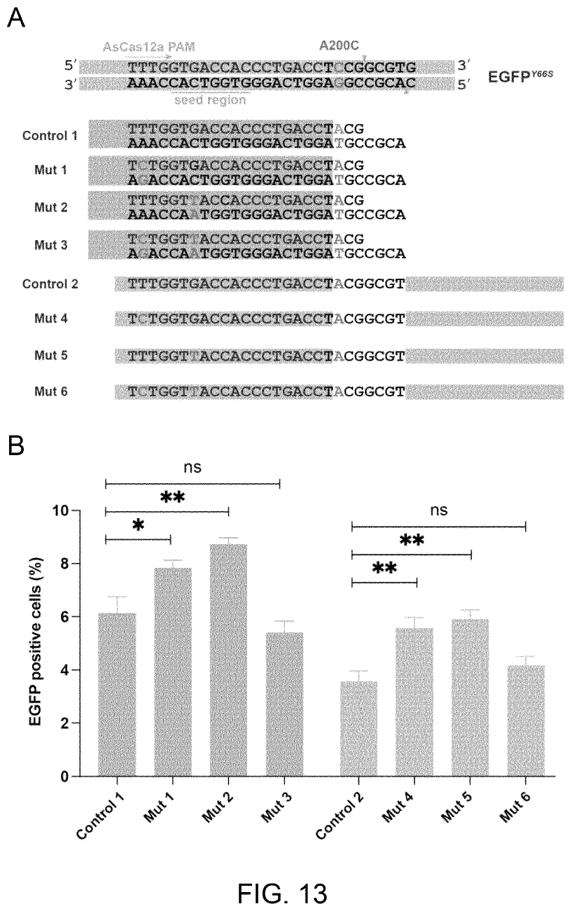

FIG. 13: Improving LAHR efficiency by PAM sequence or seed region disruption. (A) In this study, we used the EGFP^{Y66S} reporter cell line described above. The targeting locus was presented as a stretch of gray-shadowed dsDNA, where the A200C mutation was shown as a red C/G pair, the AsCas12a PAM sequence was indicated by a green arrow on top, and the seed region was green-underlined. To examine the effects of PAM sequence or seed region disruption on LAHR editing efficiency, we designed the LAHR templates containing silent mutations to destroy PAM sequence (Mut 1), seed region (Mut 2) and both (Mut 3). The ‘Control 1’ template was a normal LAHR template with untouched PAM and seed sequences. In addition, as comparison, we also included ssODN templates containing same mutations as those in the LAHR templates, which were ‘Mut 4’ with mutated PAM, ‘Mut 5’ with mutated seed and ‘Mut 6’ with both. The ‘Control 2’ template was an unmutated ssODN template. (B) The corresponding A200C repair efficiencies were indicated by the percentage of EGFP positive cells based on FACS analysis. Error bars corresponded to the standard deviation of the average of n=3 parallel samples.

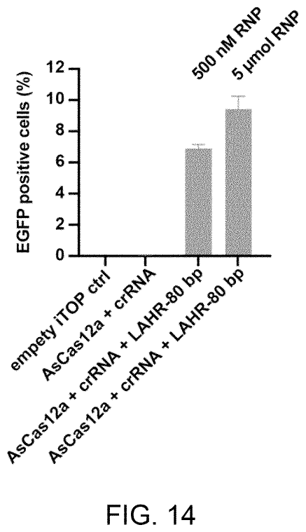

FIG. 14: Deliver Cas12a RNP and LAHR template with electroporation. To examine the applicability of LAHR with a non-iTOP delivery method, we electroporated AsCas12a RNP and LAHR template into the single-copy EGFP^{Y66S} cells. We tested the LAHR efficiency in two quantity setups, 50 pmol and 500 pmol, and the molar ratio between components is 1:1. The graph depicts the percentage of EGFP-positive cells from the FACS analysis. Error bars corresponded to the standard deviation of the average of n=3 parallel samples. The experiment was repeated three times and a representative dataset was presented here.

FIG. 15: Introducing single nucleotide substitutions in endogenous genes by LAHR and HDR. (A) A single nucleotide substitution, C698874T, was introduced in human ALK by LAHR or Cas9-mediated HDR. The editing locus, between 698861 and 698898, was in the intron 19 of the ALK gene, and was shown in double-stranded DNA. The AsCas12a PAM was indicated by a green arrow. Green arrow heads indicated the AsCas12a cleavage sites. The yellow-shadowed base pair was the substitution target. The SpCas9 PAM was indicated by a blue arrow. The alignments below the scheme were the NGS results. Guide RNA targeting sequences were underlined by grey bars. The C698874T substitution was highlighted by a blue box. The percentage of the total reads and the number of reads (in brackets) were shown at the end of each edited sequence. (B) A single nucleotide substitution, A474580G, was introduced in human CACNA1D by LAHR or Cas9-mediated HDR. The editing locus, between 474555 and 474591, was in the exon 43 of the CACNA1 D gene, and was shown in double-stranded DNA. The AsCas12a PAM was indicated by a green arrow. Green arrow heads indicated the AsCas12a cleavage sites. The yellow-shadowed base pair was the substitution target. The SpCas9 PAM was indicated by a blue arrow. The alignments below the scheme were the NGS results. Guide RNA targeting sequences were underlined by grey bars. The A474580G substitution was highlighted by a blue box. The percentage of the total reads and the number of reads (in brackets) was shown at the end of each edited sequence.

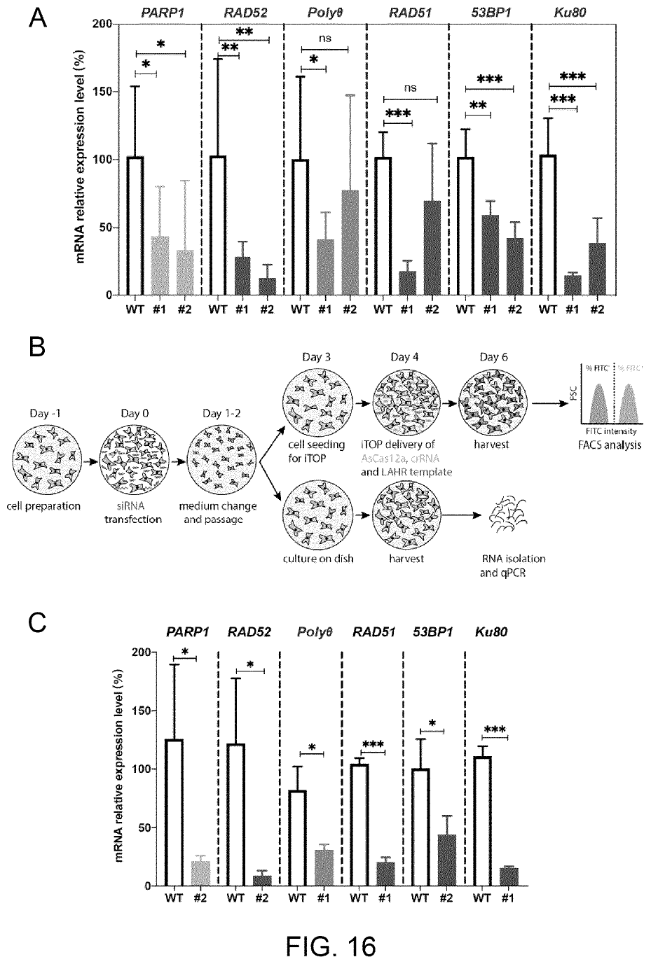

FIG. 16: (A) Selection of the siRNA performing efficient knockdown. For each target gene, two siRNAs (#1 and #2) were designed. The bar graphs exhibited RNA expression level (ΔΔCq expression level by qPCR analysis (3)) of each target gene 48 hours after transfection. ‘WT’ was the RNA expression level in non-transfected cells. The Cq value was the PCR cycle number at which the quantity of the sample amplicon reached the signal detection threshold. Error bars corresponded to the standard deviation of the average of 3 biological replicate groups, in each of which 3 technical replicates were included. The experiment was repeated three times and a representative dataset was presented here. Statistical test: two-tailed unpaired t-test, ns P>0.05, * P<0.05, ** P<0.01, *** P<0.001. (B) Implementation of LAHR following the siRNA knockdowns. A schematic presented the workflow of the siRNA knockdown, where iTOP transduction and the following analyses were based on the EGFP^{Y66S} reporter HAP1 cells. In brief, siRNA transfections were performed on day 0. On day 3, each siRNA-transfected sample was passage into two plates. Cells from one group of plates were prepared for the iTOP transduction on day 4, and the following FACS analysis on day 6 to depict the repair efficiencies under different knockdown conditions, while cells on the other group of plates were cultured till day 6 for RNA isolation and the qPCR analysis to confirm the knockdown efficacies of the target genes at the time point when LAHR were performed. (C) Confirm the effective siRNA knockdown by qPCR. As described in (B), qPCR analysis was performed to confirm the knockdown efficacy of each target gene at the time point when LAHR components were delivered. For each target gene, the selected siRNA from (A) was transfected. The bar graphs exhibited the RNA expression level (ΔΔCq expression level). ‘WT’ was the RNA expression level in non-transfected cells. Error bars corresponded to the standard deviation of the average of 3 technical replicates. Statistical test: two-tailed unpaired t-test, * P<0.05, *** P<0.001.

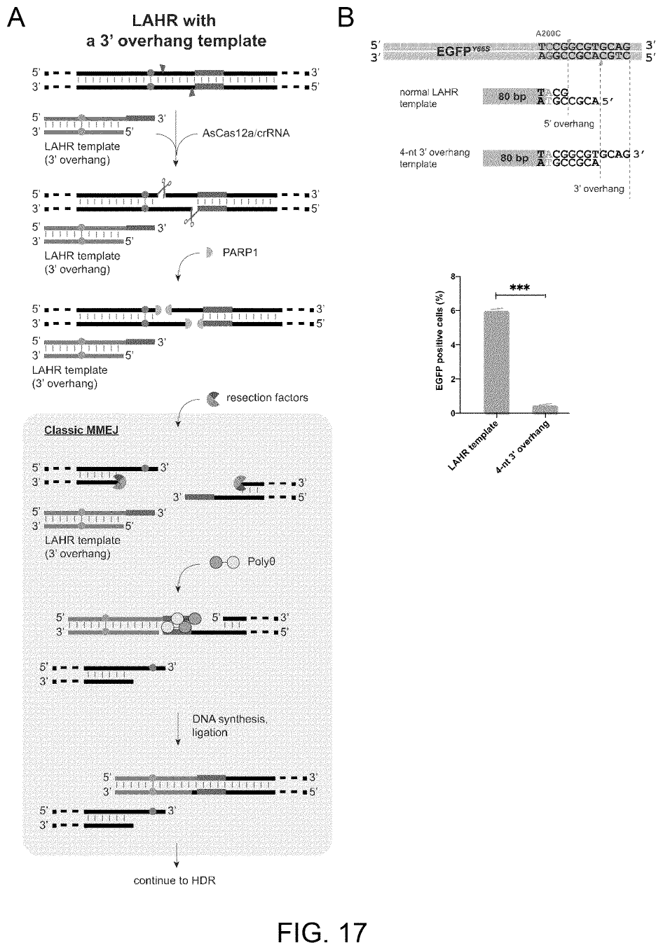

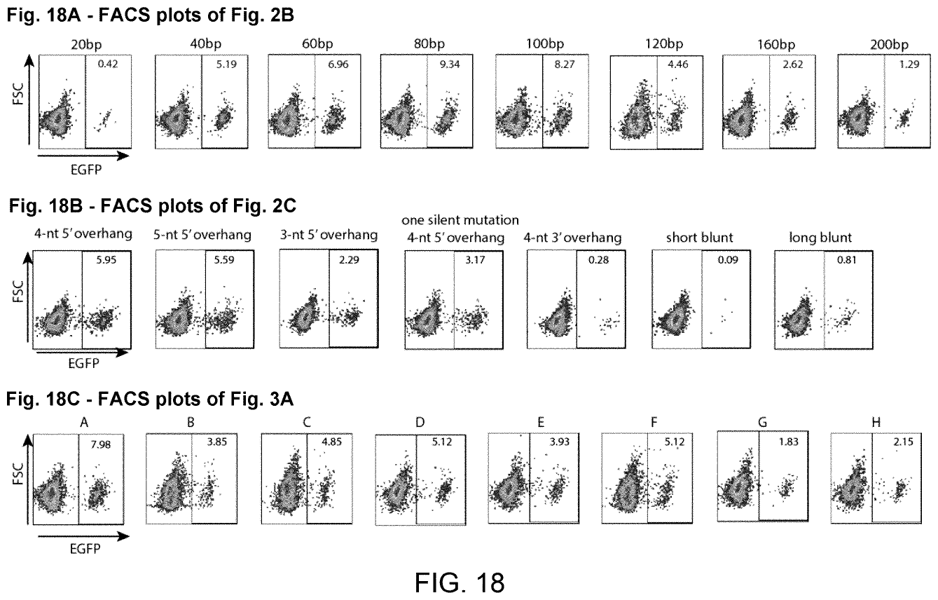

FIG. 17: Repair the EGFP^{Y66S} mutant by LAHR following a classic-MMEJ pathway. (A) A scheme presented a LAHR template containing a 3′ homologous overhang could force the ligation step of LAHR to follow a classic MMEJ pathway. The targeting locus was presented as a stretch of dsDNA which contained a mutation (red dots on both strands), an AsCas12a cut site (indicated by purple arrow heads) and a homologous region (brown blocks). An LAHR template (with a 3′ overhang) contained a substitution base pair (green dots on both strands) and a 3′ homologous overhang (a brown block). After AsCas12a cleavage, the homologous region on the editing locus was double-stranded, which could match the ‘pre-exist’ 3′ homologous overhang on the repair template only if a compatible 3′ homologous overhang exposed by resection. In the green box, a classic MMEJ pathway was presented. (B) An LAHR template containing a 3′ homologous overhang was used to repair the EGFP^{Y66S} mutation. A normal LAHR template was used as a control. The bar chart showed the comparison of the repair efficiencies depicted by the percentages of the EGFP positive cells (from FACS analysis). Error bars corresponded to the standard deviation of the average of n=3 parallel samples. The experiment was repeated three times and a representative dataset was presented here. Statistical test: two-tailed unpaired t-test, *** P<0.001.

FIG. 18: FACS plots of FIG. 2, 3, 4 and FIG. 12

### MATERIALS AND METHODS

### Reagent Sharing

Recombinant SpCas9 and AsCas12a proteins (Table 11), as well as single-copy EGFPΔfluor and EGFPY66S reporter HAP1 cell lines and Beta-2-microglobulin (B2M)-deficient HAP1 cell line (HAP1B2M−/−) are available through Divvly (https://divvly.com/geijsenlab).

### Cell Lines and Cell Culture

HAP1 cells derived from the KBM-7 cell line were a main cell line used in this study (32). All the reporter cell lines based on HAP1 cells were cultured in Iscove's Modified Dulbecco's Medium (IMDM) (Gibco), supplemented with 10% fetal bovine serum and 1% penicillin/streptomycin; HEK293 cells were cultured in Dulbecco's Modified Eagle Medium (DMEM) (Gibco), with 10% fetal bovine serum and 1% penicillin/streptomycin; C2C12 cells were cultured in DMEM, with 15% fetal bovine serum and 1% penicillin/streptomycin; ARPE19 cells were cultured in DMEM/F12 (Gibco), with 20% fetal bovine serum, 56 mM sodium bicarbonate and 2 mM L-glutamine. All cells were grown at 37° C. in a humidified atmosphere containing 5% CO2.

### Molecular Cloning

Two targeting constructs, pAAVS1-EGFPΔfluor and pAAVS1-EGFPY66S, were made to generate the single-copy EGFPΔfluor and EGFPY66S reporter HAP1 cell lines (FIG. 7). We used previously published plasmid ‘CRISPR-SP-Cas9 Reporter’ (Addgene #62733) as backbone (33). The reporter EGFP mutants, EGFPΔfluor and EGFPY66S, were synthesized as gBlock gene fragments (IDT, Table 2), and amplified by PCR using the primer pair: Fw 5′-ATGGTGAGCAAGGGC GAGG-3′ (SEQ ID NO: 1), Rv 5′-TTACTTGTACAGCTCGTCCATGCC-3′ (SEQ ID NO: 2); The homologous arms were amplified by PCR from genomic DNA of the host HAP1 cells, with the primer pairs: Fw 5′-GCTCAGTCTGGTCTATCTGCC-3′ (SEQ ID NO: 3) and Rv 5′-TGTCCCTAGTGGCCCCAC-3′ (SEQ ID NO: 4) for the left homologous arm (1011 bp); Fw 5′-GGATTGGTGACAGAAAAGCCC-3′ (SEQ ID NO: 5) and Rv 5′-TCCCCTGCTTCTTGGCC-3′ (SEQ ID NO: 6) for the right homologous arm (1107 bp). The minimal ubiquitous chromatin opening element (UCOE) fragment was amplified by PCR from the plasmid pMH0001 (Addgene #85969) (34), with the primer pair: Fw 5′-ATCGAATTCGGGAGGTGGTCC-3′ (SEQ ID NO: 7), Rv 5′-AGGACTCCGCGCCTACAG-3′ (SEQ ID NO: 8). The EGFP mutants were cloned into the backbone plasmid between NotI and BamHI sites. The left homologous arm and the minimal UCOE fragment were cloned into SpeI site upstream of the human EF1 alpha promoter. A polyA sequence (79 bp) and the right homologous arm were cloned downstream of puromycin resistance gene using ClaI site.

The expression plasmid pET15B_AsCas12a was constructed using a previously published SpCas9 expression plasmid ‘Sp-Cas9’ (Addgene #62731) as backbone (33). Briefly, E. coli. codon optimized AsCas12a coding sequence (including NLS and 6×HIS tag at C-terminal) was synthesized by GenScript (GenScript). The AsCas12a coding sequence then was amplified by PCR with the primer pair: Fw 5′-AGGAGATATACCATGACCCAGTTTG-3′ (SEQ ID NO: 9), Rv 5′-GTTAGCAGCCGGATCCTTAATG-3′ (SEQ ID NO: 10), and cloned into the backbone plasmid between NcoI and BamHI sites.

All the restriction enzymes used here were products of New England Biolabs (NEB). All PCR fragments were cloned into the backbone plasmids with In-Fusion HD Cloning Plus kit (Takara).

### Generation of Single-Copy EGFP Mutants Reporter Cell Lines

To generate the single-copy EGFPΔfluor and EGFPY66S reporter cell lines, we targeted the reporter genes into the genome of HAP1 cells at the human AAVS1 locus that located in the first intron of human PPP1R12C gene (34). To enhance the efficiency of HDR, we co-transfected the donor plasmid together with recombinant SpCas9 protein and AAVS1-T2 guide RNA (10) into the cells by using Lonza Nucleofection system following the manufacturer's protocol. In brief, 4 μg of donor plasmid, 150 pmol of recombinant SpCa9 protein (75 μM) and 300 pmol of Alt-R 2-part guide RNA (100 μM) (IDT) were added into 100 μL of Lonza Nucleofection buffer for cell line (Lonza). 1×10⁶ HAP1 cells were resuspended with the complete Lonza Nucleofection buffer, and the nucleofection was performed in a Lonza Nucleofector 2b device with the program of ‘Cell-line T-030’. 24 hours after transfection, the addition of puromycin (1:20000) was applied in the cell culture to start the positive selection. The concentration of puromycin was doubled after 3 days. After 10 days of positive selection, survival cells were single-cell sorted onto a 96-well plate. We typically sorted 96 single cells for each targeting. The correctly targeted HAP1 clones were verified by border PCRs (FIG. 7). All primers were listed in (Table 1)

### Expression and Purification of Recombinant AsCas12a Protein

To express and purify the recombinant AsCas12a protein, we adapted a previously published method (33). In brief, the expression plasmid pET15B_AsCas12a was introduced into the One Shot BL21(DE3) chemically competent E. coli. cells (Invitrogen) that were previously transformed with a chaperone plasmid pG-Tf2 (Takara). A single colony was grown overnight in 50 mL LB medium pre-culture containing 150 μg/mL ampicillin, 34 μg/mL chloramphenicol and 0.1% glucose, at 37° C., with shaking at 225 rpm. 10 mL pre-culture was then added into 400 mL of LB medium (150 μg/mL ampicillin, 34 μg/mL chloramphenicol, 1% glucose, 5 ng/mL tetracycline, and 2.5 mM MgCl₂) and cultured at 37° C., with shaking at 225 rpm until OD reached 0.5. After IPTG was added to a final concentration of 1 mM, the culture was incubated overnight at 25° C. with shaking at 225 rpm. Harvested cells were lysed in the lysis buffer (50 mM NaH₂PO₄, 1 M NaCl, 1 mM MgCl₂, 0.2 mM PMSF, 10 mM beta-2-mercaptoethanol and 0.1 mg/mL lysozyme, pH 8.0, supplemented with cOmplete Protease Inhibitor Cocktail Tablets (Roche), 1 tablet/50 mL and Benzonase Nuclease, 25 U/mL) with sonication at 4° C. The sonicated cell lysate was solubilized with the NDSB buffer (50 mM NaH₂PO₄, 1 M NaCl, 2 M NDSB-201, 2.5 mM MgCl₂ and 10 mM beta-2-mercaptoethanol, pH 8.0) at 4° C. with rotation. The solubilized cell lysate was cleared by centrifugation at 10,000×g for 60 minutes at 4° C. The Ni²⁺ affinity column chromatography was performed using a 5-mL HisTrap™ HP column with an ÄKTA pure 25 FPLC system (GE Healthcare). AsCas12a protein was eluted in the elution buffer (50 mM NaH₂PO₄, 1 M NaCl, 500 mM GABA, 500 mM imidazole, 2.5 mM MgCl₂ and 5 mM beta-2-mercaptoethanol, pH 8.0) with a continuous concentration gradient. The target elution peak was buffer exchanged into the protein storage buffer (25 mM NaH₂PO₄, 500 mM NaCl, 250 mM, 150 mM glycerol, 75 mM glycine, 1.25 mM MgCl₂, 2 mM beta-2-mercaptoethanol, pH 8.0) (33), using a HiLoad 26/600 Superdex 200 gel filtration column (GE Healthcare). The purified AsCas12a protein then was concentrated to 75 μM using Amicon Ultracel Centrifugal Filters (MWCO 100 kDa) (Millipore).

### Guide RNAs and Repair Donors Used in CAPR and LAHR

All guide RNAs used in this study are synthetic guide RNAs (IDT, Table 3). The dsDNA repair inserts (used in CAPR), or templates (used in LAHR) were produced by annealing two reverse complement ssDNA oligos. All ssDNA sequences were from Integrated DNA Technologies (IDT) and listed in Tables 4-7. Each ssDNA oligo was dissolved in the oligo annealing buffer (30 mM HEPES, pH 7.5; 100 mM potassium acetate) to reach the concentration of 100 μM. A pair of oligos for annealing was mixed in equal volume, and heat at 95° C. for 5 minutes, and cool down to room temperature.

Induced Transduction by Osmocytosis and Propanebetaine (iTOP)

The recombinant CRISPR nuclease proteins, guide RNAs and repair donors were simultaneously transduced into target cells by using the iTOP method we described previously (33). One day prior to transduction, the reporter cells were plated in the Matrigel-coated wells on 96-well plates at 30-40% confluence, such that on the day of transduction, cells were at 70-80% confluence. Next day, for each well of the 96-well plate, 50 μL of iTOP mixture that contains 20 μL of transduction supplement (Opti-MEM media supplemented with 542 mM NaCl, 333 mM GABA, 1.67×N2, 1.67×B27, 1.67×non-essential amino acids, 3.3 mM Glutamine, 167 ng/mL bFGF2, and 84 ng/mL EGF), 10 μL of CRISPR nuclease protein (75 μM), 10 μL of guide RNA (75 μM) and the excess volume of nuclease-free water to reach the 50-μL total volume, were prepared. For the no-protein control, 10 μL of protein storage buffer was used instead of the CRISPR nuclease protein; and for the no-guide control, the equal volume of nuclease-free water was used to replace the guide RNA. The 50-μL iTOP mixture was added onto the cells immediately after the culture medium was removed. The plate then was incubated in a cell culture incubator for 45 minutes, after which the iTOP mixture was gently removed and exchanged for 200 μL of regular culture medium.

### Electroporation

To deliver the LAHR components into reporter cells by electroporation, we used a Lonza Nucleofector system which includes Cell Line Nucleofector Kit V and Nucleofector 2b Device (Lonza), following the manufacturing protocol. In brief, 1 million target cells were re-suspended in 100 μL of supplemented Nucleofector solution V buffer which contains AsCas12a RNP together with repair templates (50-500 pmol of each component in the molar ratio of 1:1 were used in experiments). The electroporation was performed with the program ‘Cell-line T-020’ in the Nucleofector 2b Device. After electroporation, the cells were incubated at 37° C. and the culture medium was changed after 16 hours.

### FACS Analysis

To verify the gene editing efficacies in single-copy EGFPΔfluor and EGFPY66S reporter HAP1 cell lines, FACS analyses were performed 48 hours after iTOP transduction. Cells in each well were trypsinized and resuspended in 200 μL of FACS buffer (5% FBS in 1×DPBS) containing 1:1000 DAPI (4′,6-diamidino-2-phenylindole) DNA dye (Sigma). For the beta-2 microglobulin (B2M)-deficient HAP1 cells, 48 hours after iTOP transduction, cells from each well were firstly trypsinized and then incubated in 50 μL of staining solution (1% FITC-conjugated anti-human HLA-A, B, C antibody (Biolegend) in FACS buffer) for 10 minutes at 4° C. After washing three times with 1×DPBS, cells were resuspended in 150 μL FACS buffer containing 1:1000 DAPI DNA dye. FACS analyses were carried out on a CytoFLEX LX system (Beckman). In all experiments, the total number of 10,000 viable single cells were acquired and were gated based on side and forward light-scatter parameters. Constitutive EGFP-expressing control HAP1 cells were used to adjust the parameters for the identification and gating of EGFP/FITC positive cells. The EGFP/FITC signal was detected using the 488 nm diode laser for excitation and the 525/40 nm filter for emission.

### Cell Viability Assay

Cell viability was analyzed using an MTS Assay Kit (Abcam) following the manufacturer's instructions. In Brief, cells were seeded on a 96-well plate at 30-40% of confluence, iTOP transduction was performed when the confluence reached 70-80%. 12-24 hours after the iTOP transduction, 5 μg/mL of 3-(4,5-dimethylthiazol-2-yl)-5-(3-carboxymethoxyphenyl)-2-(4-sulfophenyl)-2H-tetrazolium (MTS reagent) was added into each well and incubated at 37° C. for 90 minutes. The absorbance was measured on a BIO-RAD XMark Microplate spectrophotometer at 490 nm (BIO-RAD).

### RNA Interference

To perform the small interfering RNA (siRNA)-mediated knockdown of the target genes involved in different DNA DSB repair pathways, the EGFPY66S reporter HAP1 cells were plated on 48-well plates and transfected with 3 pmol of either the targeting or control siRNAs (Table 8) using Lipofectamine RNAiMAX Transfection Reagent based on the manufacturer's protocol (Thermo Fisher). All siRNA oligos in this study were ordered from Thermo Fisher (Thermo Fisher).

qPCR Analysis

For siRNA targeted cell samples, total RNA was extracted using Trizol Reagent following the manufacturer's protocol. cDNA was produced using 250 ng of random hexamer primer (Invitrogen) and 5 μg of DNase-free RNase-treated total RNA each sample with SuperScript III Reverse Transcription Kit following the manufacturer's protocol (Invitrogen). qPCR was performed with iQ SYBR Green Supermix (BIO-RAD) in a BIO-RAD CFX96 Real-Time system (BIO-RAD). All the gene specific primers were listed in Table 8.

### T7 Endonuclease I Assay

To assess the AsCas12a cleavage efficiencies of the target sites in the EGFPΔfluor mutant. We applied T7 Endonuclease I (T7E1) assay following the AsCas12a targeting cleavage conducted by iTOP. Three days after iTOP AsCas12 SNP transduction, cells were harvested to isolate genomic DNA using DNeasy Blood & Tissue Kit (Qiagen). Primers used for genomic DNA amplification are listed in Table 9. The gel-purified PCR products were then subjected to T7E1 assay with Alt-R Genome Editing Detection Kit (IDT) following the manufacturing protocol. Briefly, in a thermocycler, 500 ng of purified PCR product were denatured at 95° C. for 5 min and re-annealed at −2° C. per second temperature ramp to 85° C., followed by a −0.1° C. per second ramp to 25° C., and cooled to 4° C. The rehybridized PCR product was incubated with 3 U T7E1 enzyme at 37° C. for 30 min. The enzyme-treated products were resolved on a 2% agarose gel. Densitometry analysis was performed with ImageJ (35).

### Next-Generation Sequencing

Amplicon sequencing with Illumina MiSeq platform was performed as previously described (36). In brief, the amplicon libraries were built following a two-round PCR protocol. The first round of PCR (PCR 1) amplified the target genomic loci by using locus-specific primer pairs tailed with Illumina sequencing adapters (Table 10). PCR 1 was performed using a Q5 High-Fidelity PCR Kit (NEB), following the manufacturing protocol. Each PCR 1 reaction (50 μL) contained 50 ng of genomic DNA template, 0.5 μM of each primer, 200 μM of dNTP, 0.02 U/μL of Q5 High-Fidelity DNA polymerase and 1×Q5 reaction buffer. The PCR 1 amplification initiated with a denaturation step at 98° C. for 2 min, followed by 30 cycles of denaturation at 98° C. for 10 s, primer annealing at 61° C. for 30 s, and primer extension at 72° C. for 30 s. Upon completion of the cycling steps, a final extension at 72° C. for 5 min was done and then the reaction was held at 12° C. The gel-purified PCR 1 products were then used as the templates of the second round PCR (PCR 2) where the PCR 1 products were indexed by the amplification using unique illumine barcoding primers. PCR 2 was as well performed with the Q5 High-Fidelity PCR Kit, in a 25-μL setup using 10 ng of purified PCR 1 product as template in each reaction. For the PCR 2 amplification, a denaturation step initiated at 98° C. for 12 s, followed by 12 cycles of denaturation at 98° C. for 10 s, primer annealing at 61° C. for 30 s, and primer extension at 72° C. for 30 s. When the final extension at 72° C. for 5 min was done the reaction was held at 12° C. Next, gel-purified PCR 2 products (pooled amplicons) were sequenced on an Illumina MiSeq platform, by which we generated about 30,000 total reads for each experimental sample. Sequencing reads were demultiplexed using MiSeq Reporter (Illumina). Alignment of amplicon sequences to a reference sequence was performed using CRISPResso2 (37). The editing efficiency was calculated as: the percentage of [the number of reads of edited]/[the number of total reads].

### Results

### Cut-And-Paste Repair (CAPR) Utilizing Sticky Ends Generated by AsCas12a Cleavage

As shown in FIG. 1A, our initial strategy was to try and take advantage of 5′ overhangs introduced by AsCas12a to ligate a dsDNA fragment possessing the complementary ends (repair insert) in a ‘cut-and-paste’ fashion (FIG. 1A). We referred to this strategy as ‘Cut-And-Paste Repair’ (CAPR), and it formed the basis of our initial design and subsequent refinements, as described below. For our experimental setup, we built a cell-line carrying a single-copy, fluorescence-impaired EGFP reporter (EGFP^{Δfluor}) (FIG. 7), in which the codons encoding threonine (T) 65, tyrosine (Y) 66 and glycine (G) 67, were deleted to abrogate the EGFP fluorescence. Furthermore, the EGFP^{Δfluor} construct harbored two additional silent mutations, G138A and C204T, to introduce two AsCas12a PAM sites (TTTV) flanking the deletion site (FIG. 1B). The resulting EGFP^{Δfluor} cell-line now allowed AsCas12a-mediated targeting and removal of a 115-bp region containing the Δfluor deletion, exposing two sticky ends that were compatible with a simultaneously transduced repair insert. Correct ligation of the repair insert would restore EGFP fluorescence and could be quantified by FACS analysis.

We previously reported how a combination of small molecules could trigger the efficient uptake and intracellular release of recombinant protein and small oligonucleotides, a method termed iTOP (33). We employed iTOP to simultaneously deliver all the components of CAPR (recombinant AsCas12a protein, crRNA pair, and the repair insert) into the EGFP^{Δfluor} reporter cells. The repair efficiency was quantified by FACS analysis and Sanger sequencing 48 hours after the iTOP transduction. We observed that CAPR enabled the replacement of mutated region between two cut sites and rescued the EGFP fluorescence, yet at rather low efficiency (<0.5%) (FIG. 1C). Unexpectedly, in the negative controls in which only a single left- or right-side cut was made, EGFP fluorescence was restored as well, and the one with the single right-side cut resulted in a more than 9-fold higher repair efficiency compared to CAPR (FIG. 1C). A similar trend was observed in the control group using a blunt-ended insert (FIG. 1C). Given that the sequences of both the sticky-ended and the blunt-ended repair inserts were homologous to the corresponding regions in the target reporter gene (FIG. 1D), we were intrigued by the possibility that a single sticky overhang was sufficient to trigger effective repair, potentially by combining sticky-ended ligation with HDR (homology directed repair) of the remaining template. To test if HDR was involved in the repair process, we applied a non-homologous insert together with the single-cut controls and, as expected, observed no fluorescence rescue (FIG. 1C). Nonetheless, when this non-homologous repair insert was used in combination with both guide crRNAs, this resulted in rescue of fluorescence, demonstrating that our non-homologous repair insert can repair the target sequence by the CAPR mechanism (FIG. 1C, FIG. 8A). While both blunt- and sticky-ended repair inserts could rescue the fluorescence in the single cut scenario, the blunt-ended insert yielded a lower repair efficiency since it did not match the 5′ overhang created by AsCas12a, whereas the insert with a matching sticky end favored the repair efficiency (FIG. 1C). An explanation for the observation that the ‘right cut only’ exhibited higher repair efficiency than the ‘left cut only’ condition, could be that in the ‘right cut only’ situation (FIG. 1D), the homologous arm of the repair insert (80 bp) is much longer compared to the ‘left cut only’ situation (30 bp). An alternative explanation could be however, a difference in AsCas12a cutting efficiencies at the left and right cut sites. To exclude this possibility, we determined the indel frequencies at both cut sites after AsCas12a cleavage (FIG. 8B). We observed that the AsCas12a cutting efficiencies on these two sites were similar, excluding the possibility that the observed repair differences were caused by differences in AsCas12a cleavage activity at these sites.

Taken together, our data suggested that a single sticky end generated by AsCas12a cleavage was able to ligate to the compatible end of the repair insert by end-joining mechanisms, thereby allowing the homologous region of the insert to recombine to the corresponding region on the genome by a homology-directed process (FIG. 1D). The mechanism and factors that impact this possible ‘Ligation-assisted Homologous Recombination’ (LAHR), was explored further, as outlined below.

### The Sticky End and the Homologous Arm are Indispensable for the LAHR Template

To explore the LAHR hypothesis further, we built another single copy EGFP mutant reporter (EGFP^{Y66S}) cell line (FIG. 7), in which a missense mutation A200C converted the EGFP tyrosine (Y) 66 into a serine (S), thereby eliminating the EGFP fluorescence (38). The EGFP^{Y66S} gene construct also featured two additional silent mutations, C180T and C181T, to introduce a single AsCas12a PAM site just upstream of the A200C mutation (FIG. 2A). To repair the A200C mutation and restore EGFP fluorescence, LAHR templates were designed to contain the following elements (FIG. 2A, ‘LAHR template’): (1) a sticky end that matches the PAM-distal sticky end of the AsCas12a-generated DSB ends on the target reporter gene, (2) an A/T base pair located on the homologous arm that can repair the A200C mutation (FIG. 2A, green ‘A/T’ pair) and (3) a homologous arm that shared the homology with the corresponding region adjacent to the PAM-proximal end of the AsCas12a-generated DSB (FIG. 2A, green rectangular).

To evaluate the role of the length of the homologous arm in the LAHR process, we designed a series of eight LAHR templates sharing the same sticky end, and with different lengths of the homologous arms, varying from 20 bp to 200 bp (FIG. 2B). The LAHR template together with the AsCas12a RNP were transduced into the EGFP^{Y66S} reporter cells by iTOP, followed by FACS analysis to quantify gene editing efficiency. We observed that the length of homologous arm was an important determinant of LAHR efficiency, and that 80 bp represented an optimal length in this case (FIG. 2B). Shorter homologous arms, especially the 20-bp one showed low repair efficiency, suggesting that a short homologous arm was ineffective in driving the homologous recombination-mediated integration of the distal end of the LAHR template. On the other hand, templates with lengths over 120 bp showed decreased repair efficiencies likely due to a decreased ability to diffuse into the nucleus.

Next, we explored whether the presence of a compatible sticky end on the LAHR template was required in the LAHR process. Since the optimal length of the homologous arm has been determined to be 80 bp (FIG. 2B), we generated LAHR templates with the same 80-bp homologous arm and varied the 3′ terminal ends (FIG. 2C). We observed that a template with a 4-nt 5′ overhang that perfectly matched the PAM-distal sticky end generated by AsCas12a resulted in the highest repair efficiency. LAHR templates with 4-nt or 5-nt 5′ overhangs demonstrated similar repair efficiencies, which reflected the ability of AsCas12a to cleave the non-target DNA strand at either the 18^{th} or the 19^{th} base behind the PAM sequence, yielding a 5-nt or a 4-nt 5′ overhang respectively (16). The templates with a 3-nt 5′ overhang or with a single nucleotide-mismatched 4-nt 5′ overhang (introducing a silent mutation) could still repair the mutation albeit with lower efficiency. In contrast, blunt-ended or 3′-overhang sticky-ended LAHR templates that did not match the AsCas12a-generated sticky end at all exhibited extremely low repair efficiencies (FIG. 2C). Taken together, our results indicated that both an appropriate homologous arm and a compatible sticky end were required to achieve LAHR.

### Characterization of LAHR

Since the iTOP transduction technology allowed simultaneous delivery of AsCas12a protein, crRNA, and LAHR template, we examined how the quantitative ratio of these components to affect LAHR efficiency. We had previously noticed that editing efficiencies plateaued when the concentration of SpCas9 protein reached 15-20 μM (not shown). We observed that the amount of AsCas12a protein used in LAHR exhibited similar plateau effect when the concentration was reaching 15 μM (FIG. 9A). Next, we titrated the crRNA, at a Cas12a concentration of 15 μM. As shown in FIG. 9B, the optimal molar ratio between AsCas12a protein and crRNA was 1:4. With the optimal AsCas12a protein-crRNA ratio, we made a titration curve of the LAHR template (FIG. 9C). As shown LAHR editing efficiency was linearly correlated with the concentration of LAHR template in the transduction mixture, suggesting that the concentration of the repair template at the Cas12a target site was the rate-limiting step in LAHR-based repair. We did not observe differences in cell viability in all the test conditions (FIG. 10), excluding the possibility that our results were influenced by differences in cell viability under these different conditions.

Next, we compared LAHR efficiency with simple HDR in our single-copy EGFP^{Y66S} reporter cells. As shown in FIG. 3A, in the context of AsCas12a-induced DSB, the efficiency of LAHR using a template with an 80-bp homologous arm was significantly higher than the simple HDR using a 160-nt ssODN template (from non-target strand, FIG. 11) which provided two single-stranded homologous arms flanking the repairing nucleotide. According to published reports, commonly used ssODN repair templates for HDR are 90-100 nt (39), so we also did a similar comparison between LAHR using a template with a 50-bp homologous arm and HDR using a 100-nt ssODN template. Again, LAHR exhibited higher repair efficiency. As we also noticed that HDR using a 100-nt or 160-nt ssODN template did not give significantly different repair efficiencies (FIG. 3A), thus we used 100-nt ssODN repair templates for HDR in following experiments. In addition to comparing LAHR and HDR efficiencies at the same AsCas12a-created DSB, we also compared the efficiencies between LAHR and the SpCas9-mediated HDR in repair of the EGFP^{Y66S} mutation. In line with recent reports (40,41), we also found AsCas12a exhibited a preference for an ssODN of the non-target sequence, while SpCas9 preferred an ssODN of the target strand sequence (FIG. 11). Near the A200C mutation (FIG. 3A, indicated as a red C/G pair), there were three SpCas9 PAM sites available which could be used to conduct Cas9-mediated HDR (FIG. 3A). We would like to note that, in the current experimental setup, the condition of Cas9-mediated HDR was not fully optimized, and we can therefore not conclude that LAHR editing efficiency is higher than Cas9-mediated HDR. However, our data suggests that LAHR editing efficiency is at least comparable to Cas9-mediated HDR. In scenarios where Cas9-mediated HDR fails to achieve adequate repair efficiencies, or at loci where Cas9 PAM sites are not available, LAHR could therefore be a practical alternative approach to achieve precise gene repair.

The LAHR template featured a rather short single-sided homologous arm carrying an intended nucleotide substitution, we wondered how the location of the nucleotide substitution could influence the LAHR efficiency. To address this question, we designed another experiment based on the same single-copy EGFP^{Y66S} reporter cell line used above, in which we performed LAHR with a series of repair templates carrying not only the nucleotide substitution to repair the A200C mutation, but also an additional silent mutation distributed along the homologous arm on each LAHR template (FIG. 3B). The incorporation rate of each silent mutation would indicate its position effect. The incorporation rates of silent mutations were determined by NGS analysis. We observed that incorporation rate of silent mutations diminished the more these were located toward the blunt end of the LAHR template (FIG. 3B), which demonstrates that, as expected, a mutation is more likely to be introduced by LAHR if it is closer to the Cas12a cut site. Interestingly, when we analyzed corresponding EGFP fluorescence restoration rates by FACS, we found that silent-mutations in the middle of the homologous arm demonstrated diminished EGFP restoration efficiencies (FIG. 12, templates I-IV) compared to mutation located near the Cas12a cut site or near the blunt end of the LAHR template (FIG. 12, templates V-VIII). We also performed NGS analysis to investigate the actual repair rate of the A200C mutation corresponding to each FACS sample, which corroborated the FACS results (FIG. 12). These data suggested that mutations in the middle of the LAHR template impair efficient HDR-mediated integration resulting in lower EGFP restoration rates.

Previous reports have demonstrated that mutation disrupting the PAM sequence or seed region can avoid ‘re-cutting’ of the edited genome and increase editing efficiency (42,43). We designed a LAHR targeting strategy to test whether introduction of silent mutations disrupting the Cas12a PAM site and/or seed region on the LAHR template could similarly enhance LAHR targeting efficiency (FIG. 13A). The result demonstrated that disruption of either PAM sequence or seed region significantly enhances LAHR editing efficiency (FIG. 13B), but unexpectedly, this effect was lost when both PAM and seed sequences were disrupted (FIG. 13B). Similar results were observed when ssODN templates were used (FIGS. 13A and 13B). Possibly, mutations in both the PAM and seed sequences create multiple mismatches, disrupting the homology between the LAHR template and the target genome, which may counteract the benefits gained from avoiding ‘re-cutting’.

In addition to using iTOP to deliver the LAHR components, we assessed the applicability of LAHR with a non-iTOP delivery method. With the Lonza Nucleofection system, we applied LAHR to repair the same mutation in EGFP^{Y66S} reporter cell line. We observed that nucleofection-mediated delivery of AsCas12a RNP and LAHR template similarly allows LAHR-mediated restoration of EGFP expression (FIG. 14).

### LAHR-Mediated Precise Genome Editing Targeting Endogenous Genes

Our proof-of-concept data and characterization of LAHR in the reporter cell-line demonstrated that LAHR could efficiently repair a point-mutation in an EGFP^{Y66S} reporter system. We next compared LAHR gene editing efficiency with AsCas12a or SpCas9-mediated HDR in endogenous genes. Previously, we had introduced a homozygous nonsense mutation G4045T (Glu55-STOP) in exon 2 of the human beta-2-microglobulin (B2M) gene resulting in a B2M knockout phenotype (HAP1^{B2M-/-}, unpublished). In the absence of B2M, the MHC1 complex cannot be presented at the cell surface. In this system, restoration of surface MHC1 expression can be used to quantify the restoration of B2M expression. There is an AsCas12a PAM sequence, TTTC, 14-bp upstream of this G4045T mutation, and the AsCas12a cleavage site is 4 bp downstream from the G4045T mutation (FIG. 4A). There are also three SpCas9 PAM sites surrounding the G4045T mutation, allowing to repair the mutation by Cas9-mediated HDR as well (FIG. 4A, indicated in orange, grey and blue). We transduced recombinant AsCas12a protein, the crRNA, and the LAHR template into the HAP1^{B2M-/-} cells by iTOP. As a control, a 100-nt ssODN template was applied to perform the HDR induced by the same AsCas12a cleavage. As shown in FIG. 4B, the repair efficiency of LAHR was higher than that of the AsCas12a-mediated HDR using an ssODN template (FIG. 4B, a, b and a′, b′), consistent with the result of the comparative analysis performed in the EGFP^{Y66S} reporter cells. We also compared LAHR to SpCas9-mediated HDR utilizing the three available SpCas9 PAMs with the corresponding 100-nt ssODN templates (FIG. 4B, c, d, e, and c′, d′, e′). In the repair of this mutation, LAHR performed better than the Cas9-mediated HDR (FIG. 4B), which once more demonstrated that LAHR could deliver precise genome editing at loci where the Cas9-mediated HDR may be inefficient. Moreover, we also noticed that by applying a second round of LAHR, the end repair efficiency of LAHR was almost doubled (FIG. 4B).

In addition to repairing a targeted nonsense mutation in the endogenous B2M gene, we also assessed the ability of LAHR to precisely introduce single nucleotide substitutions in other endogenous genes. In a previously published report by Wang et al. (40), point mutations, C698874T and A474580G (FIG. 15), were introduced into the human ALK and CACNA1 D genes respectively, by either AsCas12a or SpCas9-mediated HDR using ssODN templates. Here, we introduced the same substitutions by LAHR, and compared the efficiency of LAHR to the SpCas9-mediated HDR (FIG. 15). For the substitution C698874T in ALK, LAHR exhibited above 30% higher editing efficiency compared to Cas9-mediated HDR, while for A474580G in CACNA1D, the editing efficiencies from these two methods were similar (FIG. 4C).

### Mechanisms Underlying LAHR

In the LAHR process, the AsCas12a-generated DSB is repaired by using a repair template featuring a sticky end (5′ overhang) and a short double-stranded homologous arm. Since both features are indispensable to accomplish the repair, we therefore assumed that there might be two distinct DSB repair pathways involved in the LAHR process. We hereby hypothesized that LAHR could utilize the 5′ homologous overhangs to ligate the repair template to the AsCas12a-created compatible DSB end via an MMEJ pathway and is subsequently completed by a homology-directed integration of the homologous arm (FIG. 5A). To verify this hypothesis, we examined the effect of small interfering RNA (siRNA) knockdown of select genes involved in DSB-repair on LAHR efficiency. Specifically, we performed knockdown of: 1) PARP1 which is an upstream gene involved in DSB detection and recruitment of downstream DSB repair machineries (44); 2) Polyθ which plays a pivotal role in microhomology identification and annealing in MMEJ (45); 3) RAD52 which is essential in single-strand annealing (SSA) process where RAD52 binds the 3′ overhangs created by resection to facilitate end recognition and pairing (46,47); 4) RAD51 is an essential gene in HDR, which binds to resection-created 3′ single strand and leads the strand to invade template DNA based on homologies (48); 5) 53BP1 and 6) Ku80 are key factors in NHEJ, both of which inhibit the resection process, so that make NHEJ compete against MMEJ, SSA and HDR (49,50). The efficacy of the siRNA knockdown of these factors was confirmed by QPCR (FIG. 16). Upon siRNA knockdown of the indicated pathways in EGFP^{Y66S} reporter cells, we determined LAHR efficiency by FACS analysis of EGFP expression (FIG. 5B).

As expected, PARP1 knockdown decreased EGFP^{Y66S} repair efficiency, as the upstream inhibition DSB detection and repair machinery recruitment could fundamentally restrain all DSB repair. The knockdown of Polyθ, an MMEJ essential gene, also significantly decreased the repair efficiency, which indicated that Polyθ-mediated MMEJ likely plays an important role in the LAHR process. After MMEJ, the ligated repair template could potentially be utilized through SSA, a process coordinated by Rad52. However, RAD52 knockdown did not affect LAHR efficiency, suggesting that LAHR does not involve RAD52-dependent SSA-mediated repair. Knockdown of RAD51 resulted in a significantly decreased repair efficiency, which indicated that besides MMEJ, HDR likely was another essential pathway employed in LAHR. In addition, we also observed that knockdown of either 53BP1 or Ku80, consistently resulted in a slight but significant increase in LAHR-mediated repair, in line with the role of these factors in determining the balance between NHEJ and other resection-dependent repair pathways.

Canonical MMEJ is initiated by strand resection, which creates 3′ overhangs to expose matched microhomologies (51). In LAHR, the AsCas12a-created genomic 5′ overhang and the compatible 5′ overhangs on the repair template seem to bypass the need for resection. To further examine the possibility of a resection-independent MMEJ mechanism employed in LAHR, we designed a LAHR template containing a 4-nt 3′ homologous overhang which could be utilized in MMEJ only after resection exposing the matching homology on the genome (FIG. 17A). The repair efficiency using the 4-nt 3′ overhang template was significantly lower than the repair achieved by using a regular 5′ overhang LAHR template (FIG. 17B), indicating that the MMEJ mechanism employed in LAHR favors a pre-existing 5′ homologous overhang via a resection-independent pathway.

Together, these results clearly verified our hypothesis that both HDR and MMEJ pathways were essential for LAHR-mediated gene repair, and the MMEJ in LAHR takes place in a resection-independent manner.

TABLE 1
PCR primers for reporter clone verifications

| | |
|---|---|
| L-Fw | TGGACTTTGTCTCCTTCCCTG (SEQ ID NO: 11) |
| L-Rv | GTGGATGAATACTGCCATTTGTG (SEQ ID NO: 12) |
| R-Fw | ATGATCTGTGTGTGTTGGTTT (SEQ ID NO: 13) |
| R-Rv | AGACCTGACCCAAACCCAG (SEQ ID NO: 14) |
| W-Fw | TGGACTTTGTCTCCTTCCCTG (SEQ ID NO: 15) |
| W-Rv | TGGGGCTTTTCTGTCACCAAT (SEQ ID NO: 16) |
| Fw1 | GAAGAATCGCAAAACCAGCAAG (SEQ ID NO: 17) |
| Rv1 | ATCGAATGGATCTGTCTCTGTC (SEQ ID NO: 18) |
| Fw2 | ATTGCCACCACCTGTCAGC (SEQ ID NO: 19) |
| Rv2 | GCAGAATCCAGGTGGCAAC (SEQ ID NO: 20) |

TABLE 2
gBlock fragments of EGFP mutants

| Name | Sequence (5′ to 3′) |
|---|---|
| EGFP^{Δfluor} | GTGAAGCGGCCGCCACCATGGTGAGCAAGGGCGAGGAGCTGTTCACCGGGGTGGTG |
| | CCCATCCTGGTCGAGCTGGACGGCGACGTAAACGGCCACAAGTTCAGCGTGTCCGG |
| | CGAGGGCGAGGGCGATGCCACCTACGGCAAGCTGACCCTGAAATTCATCTGCACCA |
| | CCGGCAAGCTGCCCGTGCCCTGGCCCACCCTCGTGACCACCCTGGTGCAGTGTTTC |
| | AGCCGCTACCCCGACCACATGAAGCAGCACGACTTCTTCAAGTCCGCCATGCCCGA |
| | AGGCTACGTCCAGGAGCGCACCATCTTCTTCAAGGACGACGGCAACTACAAGACCC |
| | GCGCCGAGGTGAAGTTCGAGGGCGACACCCTGGTGAACCGCATCGAGCTGAAGGGC |
| | ATCGACTTCAAGGAGGACGGCAACATCCTGGGGCACAAGCTGGAGTACAACTACAA |
| | CAGCCACAACGTCTATATCATGGCCGACAAGCAGAAGAACGGCATCAAGGTGAACT |
| | TCAAGATCCGCCACAACATCGAGGACGGCAGCGTGCAGCTCGCCGACCACTACCAG |
| | CAGAACACCCCCATCGGCGACGGCCCCGTGCTGCTGCCCGACAACCACTACCTGAG |
| | CACCCAGTCCGCCCTGAGCAAAGACCCCAACGAGAAGCGCGATCACATGGTCCTGC |
| | TGGAGTTCGTGACCGCCGCCGGGATCACTCTCGGCATGGACGAGCTGTACAAGTAA |
| | (SEQ ID NO: 21) |
| EGFP^{Y66S} | GTGAAGCGGCCGCCACCATGGTGAGCAAGGGCGAGGAGCTGTTCACCGGGGTGGTG |
| | CCCATCCTGGTCGAGCTGGACGGCGACGTAAACGGCCACAAGTTCAGCGTGTCCGG |
| | CGAGGGCGAGGGCGATGCCACCTACGGCAAGCTGACCCTGAAGTTCATCTGCACCA |
| | CCGGCAAGCTGCCCGTGCCCTGGCCCACTTTGGTGACCACCCTGACCTCCGGCGTG |
| | CAGTGCTTCAGCCGCTACCCCGACCACATGAAGCAGCACGACTTCTTCAAGTCCGC |
| | CATGCCCGAAGGCTACGTCCAGGAGCGCACCATCTTCTTCAAGGACGACGGCAACT |
| | ACAAGACCCGCGCCGAGGTGAAGTTCGAGGGCGACACCCTGGTGAACCGCATCGAG |
| | CTGAAGGGCATCGACTTCAAGGAGGACGGCAACATCCTGGGGCACAAGCTGGAGTA |
| | CAACTACAACAGCCACAACGTCTATATCATGGCCGACAAGCAGAAGAACGGCATCA |
| | AGGTGAACTTCAAGATCCGCCACAACATCGAGGACGGCAGCGTGCAGCTCGCCGAC |
| | CACTACCAGCAGAACACCCCCATCGGCGACGGCCCCGTGCTGCTGCCCGACAACCA |
| | CTACCTGAGCACCCAGTCCGCCCTGAGCAAAGACCCCAACGAGAAGCGCGATCACA |
| | TGGTCCTGCTGGAGTTCGTGACCGCCGCCGGGATCACTCTCGGCATGGACGAGCTG |
| | TACAAGTAA (SEQ ID NO: 22) |

TABLE 3
Guide RNAs used in this study

| Name | Sequence (5′ to 3′) |
|---|---|
| AAVS1 targeting | [symbol] GUUUUAGAGCUAGAAAUAGCAAGUUAAA |
| (SpCas9) | AUAAGGCUAGUCCGUUAUCAACUUGAAAAAGUGGCACCGAGUCGGUGC |
| | (SEQ ID NO: 23) |
| CAPR-left (AsCas12a) | UAAUUUCUACUCUUGUAGAU [symbol] (SEQ |
| | ID NO: 24) |
| CAPR-right (AsCas12a) | UAAUUUCUACUCUUGUAGAU [symbol] (SEQ |
| | ID NO: 25) |
| EGFPY66S (AsCas12a) | UAAUUUCUACUCUUGUAGAU [symbol] (SEQ |
| | ID NO: 26) |
| B2M (AsCas12a) | UAAUUUCUACUCUUGUAGAU [symbol] (SEQ |
| | ID NO: 27) |
| ALK (AsCas12a) | UAAUUUCUACUCUUGUAGAU [symbol] (SEQ |
| | ID NO: 28) |
| CACNA1D (AsCas12a) | UAAUUUCUACUCUUGUAGAU [symbol] (SEQ |
| | ID NO: 29) |
| PAM1 (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 30) |
| PAM2 (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 31) |
| PAM3 (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 32) |
| B2M-PAM1 (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 33) |
| B2M-PAM2 (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 34) |
| B2M-PAM3 (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 35) |
| ALK (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 36) |
| CACNA1D (SpCas9) | [symbol] GUUUUAGAGCUAGAAAUAGCAAGUUAAA |
| | AUAAGGCUAGUCCGUUAUCAACUUGAAAAAGUGGCACCGAGUCGGUGC |
| | (SEQ ID NO: 37) |

TABLE 4
Repair inserts used in FIG. 1
EGFP fluorophore coding sequence

| Name | Sequence (5′ to 3′) |
|---|---|
| sticky-end | GCCACCTACGGCAAGCTGACCCTGAAATTCATCTGCACCACCGGCAAGCT |
| | GCCCGTGCCCTGGCCCACCCTCGTGACCACCCTGACCTACGGCGTGCAGT |
| | GTTTCAGCCGCTACCCCGACCAC (SEQ ID NO: 38) |
| | TCATGTGGTCGGGGTAGCGGCTGAAACACTGCACGCCGTAGGTCAGGGTG |
| | GTCACGAGGGTGGGCCAGGGCACGGGCAGCTTGCCGGTGGTGCAGATGAA |
| | TTTCAGGGTCAGCTTGCCGTAGG (SEQ ID NO: 39) |
| blunt-end | GCCACCTACGGCAAGCTGACCCTGAAATTCATCTGCACCACCGGCAAGCT |
| | GCCCGTGCCCTGGCCCACCCTCGTGACCACCCTGACCTACGGCGTGCAGT |
| | GTTTCAGCCGCTACCCCGACCACATGA (SEQ ID NO: 40) |
| | TCATGTGGTCGGGGTAGCGGCTGAAACACTGCACGCCGTAGGTCAGGGTG |
| | GTCACGAGGGTGGGCCAGGGCACGGGCAGCTTGCCGGTGGTGCAGATGAA |
| | TTTCAGGGTCAGCTTGCCGTAGGTGGC (SEQ ID NO: 41) |
| sticky-end | GCCACGTATGGGAAATTAACATTAAAGTTTATATGTACGACGGGAAAATT |
| nonhomologous | ACCAGTACCATGGCCAACACTAGTCACAACGTTAACCTACGGCGTACAAT |
| | ACTTTAGTCGGTATCCAGATCAT (SEQ ID NO: 42) |
| | TCATATGATCTGGATACCGACTAAAGTATTGTACGCCGTAGGTTAACGTT |
| | GTGACTAGTGTTGGCCATGGTACTGGTAATTTTCCCGTCGTACATATAAA |
| | CTTTAATGTTAATTTCCCATACG (SEQ ID NO: 43) |

TABLE 5
Repair templates used in FIG. 2
Mutation site

| Name | Sequence (5′ to 3′) |
|---|---|
| 20 bp | GGTGACCACCCTGACCTACG (SEQ ID NO: 44) |
| | ACGCCGTAGGTCAGGGTGGTCACC (SEQ ID NO: 45) |
| 40 bp | CCCGTGCCCTGGCCCACTTTGGTGACCACCCTGACCTACG (SEQ ID NO: |
| | 46) |
| | ACGCCGTAGGTCAGGGTGGTCACCAAAGTGGGCCAGGGCACGGG (SEQ ID |
| | NO: 47) |
| 60 bp | TCTGCACCACCGGCAAGCTGCCCGTGCCCTGGCCCACTTTGGTGACCACCCTGAC |
| | CTACG (SEQ ID NO: 48) |
| | CGTAGGTCAGGGTGGTCACCAAAGTGGGCCAGGGCACGGGCAGCTTGCCGGTGGT |
| | GCAGA (SEQ ID NO: 49) |
| 80 bp | CAAGCTGACCCTGAAGTTCATCTGCACCACCGGCAAGCTGCCCGTGCCCTGGCCC |
| | ACTTTGGTGACCACCCTGACCTACG (SEQ ID NO: 50) |
| | ACGCCGTAGGTCAGGGTGGTCACCAAAGTGGGCCAGGGCACGGGCAGCTTGCCGG |
| | TGGTGCAGATGAACTTCAGGGTCAGCTTG (SEQ ID NO: 51) |
| 100 bp | GAGGGCGATGCCACCTACGGCAAGCTGACCCTGAAGTTCATCTGCACCACCGGCA |
| | AGCTGCCCGTGCCCTGGCCCACTTTGGTGACCACCCTGACCTACG (SEQ ID |
| | NO: 52) |
| | ACGCCGTAGGTCAGGGTGGTCACCAAAGTGGGCCAGGGCACGGGCAGCTTGCCGG |
| | TGGTGCAGATGAACTTCAGGGTCAGCTTGCCGTAGGTGGCATCGCCCTC (SEQ |
| | ID NO: 53) |
| 120 bp | TCAGCGTGTCCGGCGAGGGCGAGGGCGATGCCACCTACGGCAAGCTGACCCTGAA |
| | GTTCATCTGCACCACCGGCAAGCTGCCCGTGCCCTGGCCCACTTTGGTGACCACC |
| | CTGACCTACG (SEQ ID NO: 54) |
| | ACGCCGTAGGTCAGGGTGGTCACCAAAGTGGGCCAGGGCACGGGCAGCTTGCCGG |
| | TGGTGCAGATGAACTTCAGGGTCAGCTTGCCGTAGGTGGCATCGCCCTCGCCCTC |
| | GCCGGACACGCTGA (SEQ ID NO: 55) |
| 160 bp | ATCCTGGTCGAGCTGGACGGCGACGTAAACGGCCACAAGTTCAGCGTGTCCGGCG |
| | AGGGCGAGGGCGATGCCACCTACGGCAAGCTGACCCTGAAGTTCATCTGCACCAC |
| | CGGCAAGCTGCCCGTGCCCTGGCCCACTTTGGTGACCACCCTGACCTACG (SEQ |
| | ID NO: 56) |
| | ACGCCGTAGGTCAGGGTGGTCACCAAAGTGGGCCAGGGCACGGGCAGCTTGCCGG |
| | TGGTGCAGATGAACTTCAGGGTCAGCTTGCCGTAGGTGGCATCGCCCTCGCCCTC |
| | GCCGGACACGCTGAACTTGTGGCCGTTTACGTCGCCGTCCAGCTCGACCAGGAT |
| | (SEQ ID NO: 57) |
| 200 bp | GGTGAGCAAGGGCGAGGAGCTGTTCACCGGGGTGGTGCCCATCCTGGTCGAGCTG |
| | GACGGCGACGTAAACGGCCACAAGTTCAGCGTGTCCGGCGAGGGCGAGGGCGATG |
| | CCACCTACGGCAAGCTGACCCTGAAGTTCATCTGCACCACCGGCAAGCTGCCCGT |
| | GCCCTGGCCCACTTTGGTGACCACCCTGACCTACG (SEQ ID NO: 58) |
| | ACGCCGTAGGTCAGGGTGGTCACCAAAGTGGGCCAGGGCACGGGCAGCTTGCCGG |
| | TGGTGCAGATGAACTTCAGGGTCAGCTTGCCGTAGGTGGCATCGCCCTCGCCCTC |
| | GCCGGACACGCTGAACTTGTGGCCGTTTACGTCGCCGTCCAGCTCGACCAGGATG |
| | GGCACCACCCCGGTGAACAGCTCCTCGCCCTTGCTCACC (SEQ ID NO: 59) |
| 4-nt 5′ | CAAGCTGACCCTGAAGTTCATCTGCACCACCGGCAAGCTGCCCGTGCCCTGGCCC |
| overhang | ACTTTGGTGACCACCCTGACCTACG (SEQ ID NO: 60) |
| | ACGCCGTAGGTCAGGGTGGTCACCAAAGTGGGCCAGGGCACGGGCAGCTTGCCGG |
| | TGGTGCAGATGAACTTCAGGGTCAGCTTG (SEQ ID NO: 61) |
| 5-nt 5′ | CAAGCTGACCCTGAAGTTCATCTGCACCACCGGCAAGCTGCCCGTGCCCTGGCCC |
| overhang | ACTTTGGTGACCACCCTGACCTAC (SEQ ID NO: 62) |
| | ACGCCGTAGGTCAGGGTGGTCACCAAAGTGGGCCAGGGCACGGGCAGCTTGCCGG |
| | TGGTGCAGATGAACTTCAGGGTCAGCTTG (SEQ ID NO: 63) |
| 3-nt 5′ | CAAGCTGACCCTGAAGTTCATCTGCACCACCGGCAAGCTGCCCGTGCCCTGGCCC |
| overhang | ACTTTGGTGACCACCCTGACCTACG (SEQ ID NO: 64) |
| | CGCCGTAGGTCAGGGTGGTCACCAAAGTGGGCCAGGGCACGGGCAGCTTGCCGGT |
| | GGTGCAGATGAACTTCAGGGTCAGCTTG (SEQ ID NO: 65) |
| 4-nt 5′ | CAAGCTGACCCTGAAGTTCATCTGCACCACCGGCAAGCTGCCCGTGCCCTGGCCC |
| overhang | ACTTTGGTGACCACCCTGACCTACG (SEQ ID NO: 66) |
| (one silent | ACTCCGTAGGTCAGGGTGGTCACCAAAGTGGGCCAGGGCACGGGCAGCTTGCCGG |
| mutation) | TGGTGCAGATGAACTTCAGGGTCAGCTTG (SEQ ID NO: 67) |
| 4-nt 3′ | CAAGCTGACCCTGAAGTTCATCTGCACCACCGGCAAGCTGCCCGTGCCCTGGCCC |
| overhang | ACTTTGGTGACCACCCTGACCTACGGCGT (SEQ ID NO: 68) |
| | CGTAGGTCAGGGTGGTCACCAAAGTGGGCCAGGGCACGGGCAGCTTGCCGGTGGT |
| | GCAGATGAACTTCAGGGTCAGCTTG (SEQ ID NO: 69) |
| Short blunt | CAAGCTGACCCTGAAGTTCATCTGCACCACCGGCAAGCTGCCCGTGCCCTGGCCC |
| | ACTTTGGTGACCACCCTGACCTACG (SEQ ID NO: 70) |
| | CGTAGGTCAGGGTGGTCACCAAAGTGGGCCAGGGCACGGGCAGCTTGCCGGTGGT |
| | GCAGATGAACTTCAGGGTCAGCTTG (SEQ ID NO: 71) |
| Long blunt | CAAGCTGACCCTGAAGTTCATCTGCACCACCGGCAAGCTGCCCGTGCCCTGGCCC |
| | ACTTTGGTGACCACCCTGACCTACGGCGT (SEQ ID NO: 72) |
| | ACGCCGTAGGTCAGGGTGGTCACCAAAGTGGGCCAGGGCACGGGCAGCTTGCCGG |
| | TGGTGCAGATGAACTTCAGGGTCAGCTTG (SEQ ID NO: 73) |
| 4-nt 3′ | CAAGCTGACCCTGAAGTTCATCTGCACCACCGGCAAGCTGCCCGTGCCCTGGCCC |
| overhang | ACTTTGGTGACCACCCTGACCTACGGCGTGCAG (SEQ ID NO: 74) |
| (FIG. 17) | ACGCCGTAGGTCAGGGTGGTCACCAAAGTGGGCCAGGGCACGGGCAGCTTGCCGG |
| | TGGTGCAGATGAACTTCAGGGTCAGCTTG (SEQ ID NO: 75) |

TABLE 6
Repair templates used in FIG. 3
Mutation site

| Name | Sequence (5′ to 3′) |
|---|---|
| a | CAAGCTGACCCTGAAGTTCATCTGCACCACCGGCAAGCTGCCCGTGCCCTGGCCCACTT |
| | TGGTGACCACCCTGACCTACG (SEQ ID NO: 76) |
| | ACGCCGTAGGTCAGGGTGGTCACCAAAGTGGGCCAGGGCACGGGCAGCTTGCCGGTGGT |
| | GCAGATGAACTTCAGGGTCAGCTTG (SEQ ID NO: 77 |
| b | CGGCAAGCTGACCCTGAAGTTCATCTGCACCACCGGCAAGCTGCCCGTGCCCTGGCCCA |
| | CTTTGGTGACCACCCTGACCTACGGCGTGCAGTGCTTCAGCCGCTACCCCGACCACATG |
| | AAGCAGCACGACTTCTTCAAGTCCGCCATGCCCGAAGGCTAC (SEQ ID NO: 78) |
| | GTAGCCTTCGGGCATGGCGGACTTGAAGAAGTCGTGCTGCTTCATGTGGTCGGGGTAGC |
| | GGCTGAAGCACTGCACGCCGTAGGTCAGGGTGGTCACCAAAGTGGGCCAGGGCACGGGC |
| | AGCTTGCCGGTGGTGCAGATGAACTTCAGGGTCAGCTTGCCG (SEQ ID NO: 79) |
| c | CGGCAAGCTGACCCTGAAGTTCATCTGCACCACCGGCAAGCTGCCCGTGCCCTGGCCCA |
| | CTTTGGTGACCACCCTGACCTACGGCGTGCAGTGCTTCAGCCGCTACCCCGACCACATG |
| | AAGCAGCACGACTTCTTCAAGTCCGCCATGCCCGAAGGCTAC (SEQ ID NO: 80) |
| d | CGGCAAGCTGCCCGTGCCCTGGCCCACTTTGGTGACCACCCTGACCTACG (SEQ ID |
| | NO: 81) |
| | ACGCCGTAGGTCAGGGTGGTCACCAAAGTGGGCCAGGGCACGGGCAGCTTGCCG (SEQ |
| | ID NO: 82) |
| e/g/h | CACCGGCAAGCTGCCCGTGCCCTGGCCCACTTTGGTGACCACCCTGACCTACGGCGTGC |
| | AGTGCTTCAGCCGCTACCCCGACCACATGAAGCAGCACGACT (SEQ ID NO: 83) |
| f | AGTCGTGCTGCTTCATGTGGTCGGGGTAGCGGCTGAAGCACTGCACGCCGTAGGTCAGG |
| | GTGGTCACCAAAGTGGGCCAGGGCACGGGCAGCTTGCCGGTG (SEQ ID NO: 84) |
| I | CAAGCTGACCCTGAAGTTCATCTGCACCACCGGCAAGCTGCCCGTGCCCTGGCCCACTT |
| | TGGTGACCACCCTGACCTACG (SEQ ID NO: 85) |
| | ACGCCGTAGGTCAGGGTGGTCACCAAAGTGGGCCAGGGCACGGGCAGCTTGCCGGTGGT |
| | GCAGATGAACTTCAGGGTCAGCTTG (SEQ ID NO: 86) |
| II | CAAGCTGACCCTGAAGTTCATCTGCACCACCGGCAAGCTGCCCGTGCCCTGGCCCACTT |
| | TGGTGACCACACTGACCTACG (SEQ ID NO: 87) |
| | ACGCCGTAGGTCAGTGTGGTCACCAAAGTGGGCCAGGGCACGGGCAGCTTGCCGGTGGT |
| | GCAGATGAACTTCAGGGTCAGCTTG (SEQ ID NO: 88) |
| III | CAAGCTGACCCTGAAGTTCATCTGCACCACCGGCAAGCTGCCCGTGCCCTGGCCCACTT |
| | AGGTGACCACCCTGACCTACG (SEQ ID NO: 89) |
| | ACGCCGTAGGTCAGGGTGGTCACCTAAGTGGGCCAGGGCACGGGCAGCTTGCCGGTGGT |
| | GCAGATGAACTTCAGGGTCAGCTTG (SEQ ID NO: 90) |
| IV | CAAGCTGACCCTGAAGTTCATCTGCACCACCGGCAAGCTGCCCGTGCCCTTGCCCACTT |
| | TGGTGACCACCCTGACCTACG (SEQ ID NO: 91) |
| | ACGCCGTAGGTCAGGGTGGTCACCAAAGTGGGCAAGGGCACGGGCAGCTTGCCGGTGGT |
| | GCAGATGAACTTCAGGGTCAGCTTG (SEQ ID NO: 92) |
| V | CAAGCTGACCCTGAAGTTCATCTGCACCACAGGCAAGCTGCCCGTGCCCTGGCCCACTT |
| | TGGTGACCACCCTGACCTACG (SEQ ID NO: 93) |
| | ACGCCGTAGGTCAGGGTGGTCACCAAAGTGGGCCAGGGCACGGGCAGCTTGCCTGTGGT |
| | GCAGATGAACTTCAGGGTCAGCTTG (SEQ ID NO: 94) |
| VI | CAAGCTGACCCTGAAGTTCATATGCACCACCGGCAAGCTGCCCGTGCCCTGGCCCACTT |
| | TGGTGACCACCCTGACCTACG (SEQ ID NO: 95) |
| | ACGCCGTAGGTCAGGGTGGTCACCAAAGTGGGCCAGGGCACGGGCAGCTTGCCGGTGGT |
| | GCATATGAACTTCAGGGTCAGCTTG (SEQ ID NO: 96) |
| VII | CAAGCTGACTCTGAAGTTCATCTGCACCACCGGCAAGCTGCCCGTGCCCTGGCCCACTT |
| | TGGTGACCACCCTGACCTACG (SEQ ID NO: 97) |
| | ACGCCGTAGGTCAGGGTGGTCACCAAAGTGGGCCAGGGCACGGGCAGCTTGCCGGTGGT |
| | GCAGATGAACTTCAGAGTCAGCTTG (SEQ ID NO: 98) |
| VIII | AAAGCTGACCCTGAAGTTCATCTGCACCACCGGCAAGCTGCCCGTGCCCTGGCCCACTT |
| | TGGTGACCACCCTGACCTACG (SEQ ID NO: 99) |
| | ACGCCGTAGGTCAGGGTGGTCACCAAAGTGGGCCAGGGCACGGGCAGCTTGCCGGTGGT |
| | GCAGATGAACTTCAGGGTCAGCTTT (SEQ ID NO: 100) |

TABLE 7
Repair templates used in FIG. 4
Mutation site

| Name | Sequence (5′ to 3′) |
|---|---|
| LAHR template | TCACGTCATCCAGCAGAGAATGGAAAGTCAAATTTCCTGAATTGCTATG |
| (B2M) | TGTCTGGGTTTCATCCATCCGACATTGAAGT (SEQ ID NO: 101) |
| | GTCAACTTCAATGTCGGATGGATGAAACCCAGACACATAGCAATTCAGG |
| | AAATTTGACTTTCCATTCTCTGCTGGATGACGTGA (SEQ ID NO: |
| | 102) |
| ssODN1 (B2M) | AGTCAAATTTCCTGAATTGCTATGTGTCTGGGTTTCATCCATCCGACAT |
| | TGAAGTTGACTTACTGAAGAATGGAGAGAGAATTGAAAAAGTGGAGCAT |
| | TC (SEQ ID NO: 103) |
| ssODN2 (B2M) | GAATGCTCCACTTTTTCAATTCTCTCTCCATTCTTCAGTAAGTCAACTT |
| | CAATGTCGGATGGATGAAACCCAGACACATAGCAATTCAGGAAATTTGA |
| | CT (SEQ ID NO: 104) |
| LAHR template | CCCCGCTTCTCGTGTTTGTCCACTAAATGTGACGCCCAGGCTCAGGACC |
| (ALK) | CCCAGCTGCCTCATTATTGTGGCCTGTTTGACTCT (SEQ ID NO: |
| | 105) |
| | AGAGTCAAACAGGCCACAATAATGAGGCAGCTGGGGGTCCTGAGCCTGG |
| | GCGTCACATTTAGTGGACAAACACGAGAAGC (SEQ ID NO: 106) |
| ssODN (ALK) | AGCAAAGCCATGTTGAGGGTATTACTCCTGAGTGTGTATGTTACCCCCG |
| | CTTCTCGTGTTTGTCCACTAAATGTGACGCCCAGGCTCAGGACCCCCAG |
| | CT (SEQ ID NO: 107) |
| LAHR template | AGACCCAGAGATACATGGCTATTTCAGGGACCCCCACTGCTTGGGGGAG |
| (CACNA1D) | CAGGAGTATTTCAGTAGTGAGGAATGCTGCG (SEQ ID NO: 108) |
| | TCCTCGCAGCATTCCTCACTACTGAAATACTCCTGCTCCCCCAAGCAGT |
| | GGGGGTCCCTGAAATAGCCATGTATCTCTGGGTCT (SEQ ID NO: |
| | 109) |
| ssODN | AGGAGCCAGAGCAGCTCACCTGCTCCAGGTGGGCGAGCTGTCATCCTCG |
| (CACNA1D) | CAGCATTCCTCACTACTGAAATACTCCTGCTCCCCCAAGCAGTGGGGGT |
| | CC (SEQ ID NO: 110) |

TABLE 8
siRNA target sequences and qPCR primers

| Name | Sequence (5′to 3′) |
|---|---|
| T-53BP1#1 | GCCAGGUUCUAGAGGAUGA (SEQ ID NO: 111) |
| T-53BP1#2 | GCACAAGAACTTATGGAAAGU (SEQ ID NO: 112) |
| T-Ku70#1 | GGAAGAGATAGUUUGAUUU (SEQ ID NO: 113) |
| T-Ku70#2 | GACAUAUCCUUGUUCUACA (SEQ ID NO: 114) |
| T-Ku80#1 | GAAGUUCUGUCACAGOUGA (SEQ ID NO: 115) |
| T-Ku80#2 | GUCUUCAAGGGUGUCUGUC (SEQ ID NO: 116) |
| T-Polyθ#1 | CCUUAAGACUGUAGGUACU (SEQ ID NO: 117) |
| T-Polyθ#2 | CAAACAAACCCUUAUCGUAAA (SEQ ID NO: 118) |
| T-PARP1#1 | AAGCCUCCGCUCCUGAACAAU (SEQ ID NO: 119) |
| T-PARP1#2 | AAGAUAGAGCGUGAAGGCGAA (SEQ ID NO: 120) |
| T-DNA ligaseIV#1 | AAGCCAGACAAAAGAGGUGAA (SEQ ID NO: 121) |
| T-DNA ligaseIV#2 | CCGUGAAUAUGAUUGCUAU (SEQ ID NO: 122) |
| T-Rad51#1 | GGGAAUUAGUGAAGCCAAA (SEQ ID NO: 123) |
| T-Rad51#2 | GAAUUGAGACUGGAUCUAU (SEQ ID NO: 124) |
| T-Rad52#1 | GGGAAUUAGUGAAGCCAAA (SEQ ID NO: 125) |
| T-Rad52#2 | AGUCCAAGGCUUUAUCUUU (SEQ ID NO: 126) |
| 53BP1#1-Fw | AGATGGACCCTACTGGAAGTC (SEQ ID NO: 127) |
| 53BP1#1-Rv | TGTTCATTGAACCCACTATTACCGTC (SEQ ID NO: 128) |
| 53BP1#2-Fw | AGCAAGGACATCCCTGTGACA (SEQ ID NO: 129) |
| 53BP1#2-Rv | ACCTCTGACCAGAGAGCTGCA (SEQ ID NO: 130) |
| Ku70#1-Fw | CAAGAAGAGAACCTTGAAGCAA (SEQ ID NO: 131) |
| Ku70#1-Rv | TGGATGCTCATGTCAAAAGGT (SEQ ID NO: 132) |
| Ku70#2-Fw | CATCTTCCTTGACTTGATGCA (SEQ ID NO: 133) |
| Ku70#2-Rv | TAGAGCTTTATTGGAGGAGGC (SEQ ID NO: 134) |
| Ku80#1-Fw | TCAGAAGAGGAGOGOTTTAACAA (SEQ ID NO: 135) |
| Ku80#1-Rv | TCGTCCACATCACCACCTTC (SEQ ID NO: 136) |
| Ku80#2-Fw | AAGACTTGCGGCAATACATG (SEQ ID NO: 137) |
| Ku80#2-Rv | CAGCATATTCCAAATATGCTGC (SEQ ID NO: 138) |
| Polyθ#1-Fw | CAACAGATGGCAACTGAAAATG (SEQ ID NO: 139) |
| Polyθ#1-Rv | CTTGTTTCAGGAACTGGAAGAC (SEQ ID NO: 140) |
| Polyθ#2-Fw | CATGACAGAGACAGTGAAGAATTG (SEQ ID NO: 141) |
| Polyθ#2-Rv | ACTTTGGAGCATACCCTCTC (SEQ ID NO: 142) |
| PARP1#1-Fw | ATACTCCATCCTCAGTGAGGTC (SEQ ID NO: 143) |
| PARP1#1-Rv | ATGGGATCCTTGCTGCTATC (SEQ ID NO: 144) |
| PARP1#2-Fw | GAGATTCTGAAGAAGCCGAGA (SEQ ID NO: 145) |
| PARP1#2-Rv | CAACTTCTOCCAACAGGATTAA (SEQ ID NO: 146) |
| DNA ligaseIV#1-Fw | CACCAATTCCAGGTAGAATAGA (SEQ ID NO: 147) |
| DNA ligaseIV#1-Rv | ACACAGATGGCTTCTCAC (SEQ ID NO: 148) |
| DNA ligaseIV#2-Fw | ATGGCAGCCTCGCTTTAT (SEQ ID NO: 149) |
| DNA ligaseIV#2-Rv | TGTGGCGTCGAAACATACTG (SEQ ID NO: 150) |
| Rad51#1-Fw | AGTGTGGCATAAATGCCAA (SEQ ID NO: 151) |
| Rad51#1-Rv | GTGAAACCCATTGGAACT (SEQ ID NO: 152) |
| Rad51#2-Fw | AGTTCCAATGGGTTTCAC (SEQ ID NO: 153) |
| Rad51#2-Rv | TGGCAGGTGACAGCTA (SEQ ID NO: 154) |
| Rad52#1-Fw | ACAGCACTCCTGTAACTGTCT (SEQ ID NO: 155) |
| Rad52#1-Rv | TGTTGTGCGTTGGTCAGCG (SEQ ID NO: 156) |
| Rad52#2-Fw | GTTGACCTCAACAATGGC (SEQ ID NO: 157) |
| Rad52#2-Rv | CTGGCGTGGAAGOTTATTTA (SEQ ID NO: 158) |

TABLE 9
Genomic PCR primers and sequencing primers

| Name | Sequence (5′ to 3′) |
|---|---|
| EGFP-Fw | GCCTCAGACAGTGGTTCAAAG |
| | (SEQ ID NO: 159) |
| EGFP-Rv | TAACATATAGACAAACGCACACCG |
| | (SEQ ID NO: 160) |
| EGFP-seq | TGAGCAAGGGCGAGGAG |
| | (SEQ ID NO: 161) |
| B2M-Fw | GAGGGAAAGATACCAAGTCACGG |
| | (SEQ ID NO: 162) |
| B2M-Rv | CATCAGTATCTCAGCAGGTGCCAC |
| | (SEQ ID NO: 163) |
| B2M-seq | TGGAAATGGAATTGGGAG |
| | (SEQ ID NO: 164) |

TABLE 10
Next generation sequencing primers

| Name | Sequence (5′ to 3′) |
|---|---|
| Miseq EGFP-Fw | GATGTGTATAAGAGACAGGACCCTGAAGTTC |
| | ATCTGCAC (SEQ ID NO: 165) |
| Miseq EGFP-Rv | CGTGTGCTCTTCCGATCTGCTCCTGGACGTA |
| | GCCTT (SEQ ID NO: 166) |
| Miseq ALK-Fw | GATGTGTATAAGAGACAGAAGAACTGGAAGC |
| | CCGA (SEQ ID NO: 167) |
| Miseq ALK-Rv | CGTGTGCTCTTCCGATCTGTCAAACAGGCCA |
| | CAATAATGAG (SEQ ID NO: 168) |
| Miseq CACNA1D- | GATGTGTATAAGAGACAGGACCCAGAGATAC |
| Fw | ATGGCTATTT (SEQ ID NO: 169) |
| Miseq CACNA1D- | CGTGTGCTCTTCCGATCTAGGGCGGCCTCCC |
| Rv | (SEQ ID NO: 170) |

TABLE 11
Protein sequence of recombinant proteins
Protein coding sequence, Nuclear localization signal and His tag

| Name | |
|---|---|
| SpCas9 | MDKKYSIGLDIGTNSVGWAVITDEYKVPSKKFKVLGNTDRHSIKKNLIGALLFDSGETAEAT |
| | RLKRTARRRYTRRKNRICYLQEIFSNEMAKVDDSFFHRLEESFLVEEDKKHERHPIFGNIVD |
| | EVAYHEKYPTIYHLRKKLVDSTDKADLRLIYLALAHMIKFRGHFLIEGDLNPDNSDVDKLFI |
| | QLVQTYNQLFEENPINASGVDAKAILSARLSKSRRLENLIAQLPGEKKNGLFGNLIALSLGL |
| | TPNFKSNFDLAEDAKLQLSKDTYDDDLDNLLAQIGDQYADLFLAAKNLSDAILLSDILRVNT |
| | EITKAPLSASMIKRYDEHHQDLTLLKALVRQQLPEKYKEIFFDQSKNGYAGYIDGGASQEEF |
| | YKFIKPILEKMDGTEELLVKLNREDLLRKQRTFDNGSIPHQIHLGELHAILRRQEDFYPFLK |
| | DNREKIEKILTFRIPYYVGPLARGNSRFAWMTRKSEETITPWNFEEVVDKGASAQSFIERMT |
| | NFDKNLPNEKVLPKHSLLYEYFTVYNELTKVKYVTEGMRKPAFLSGEQKKAIVDLLFKTNRK |
| | VTVKQLKEDYFKKIECFDSVEISGVEDRFNASLGTYHDLLKIIKDKDFLDNEENEDILEDIV |
| | LTLTLFEDREMIEERLKTYAHLFDDKVMKQLKRRRYTGWGRLSRKLINGIRDKQSGKTILDF |
| | LKSDGFANRNFMQLIHDDSLTFKEDIQKAQVSGQGDSLHEHIANLAGSPAIKKGILQTVKVV |
| | DELVKVMGRHKPENIVIEMARENQTTQKGQKNSRERMKRIEEGIKELGSQILKEHPVENTQL |
| | QNEKLYLYYLQNGRDMYVDQELDINRLSDYDVDHIVPQSFLKDDSIDNKVLTRSDKNRGKSD |
| | NVPSEEVVKKMKNYWRQLLNAKLITQRKFDNLTKAERGGLSELDKAGFIKRQLVETRQITKH |
| | VAQILDSRMNTKYDENDKLIREVKVITLKSKLVSDFRKDFQFYKVREINNYHHAHDAYLNAV |
| | VGTALIKKYPKLESEFVYGDYKVYDVRKMIAKSEQEIGKATAKYFFYSNIMNFFKTEITLAN |
| | GEIRKRPLIETNGETGEIVWDKGRDFATVRKVLSMPQVNIVKKTEVQTGGFSKESILPKRNS |
| | DKLIARKKDWDPKKYGGFDSPTVAYSVLVVAKVEKGKSKKLKSVKELLGITIMERSSFEKNP |
| | IDFLEAKGYKEVKKDLIIKLPKYSLFELENGRKRMLASAGELQKGNELALPSKYVNFLYLAS |
| | HYEKLKGSPEDNEQKQLFVEQHKHYLDEIIEQISEFSKRVILADANLDKVLSAYNKHRDKPI |
| | REQAENIIHLFTLTNLGAPAAFKYFDTTIDRKRYTSTKEVLDATLIHQSITGLYETRIDLSQ |
| | LGGDGGGSGTRLPKKKRKVGGGSHHHHHH Stop (SEQ ID NO: 171) |
| AsCas12a | MTQFEGFTNLYQVSKTLRFELIPQGKTLKHIQEQGFIEEDKARNDHYKELKPIIDRIYKTYA |
| | DQCLQLVQLDWENLSAAIDSYRKEKTEETRNALIEEQATYRNAIHDYFIGRTDNLTDAINKR |
| | HAEIYKGLFKAELFNGKVLKQLGTVTTTEHENALLRSFDKFTTYFSGFYENRKNVFSAEDIS |
| | TAIPHRIVQDNFPKFKENCHIFTRLITAVPSLREHFENVKKAIGIFVSTSIEEVFSFPFYNQ |
| | LLTQTQIDLYNQLLGGISREAGTEKIKGLNEVLNLAIQKNDETAHIIASLPHRFIPLFKQIL |
| | SDRNTLSFILEEFKSDEEVIQSFCKYKTLLRNENVLETAEALFNELNSIDLTHIFISHKKLE |
| | TISSALCDHWDTLRNALYERRISELTGKITKSAKEKVQRSLKHEDINLQEIISAAGKELSEA |
| | FKQKTSEILSHAHAALDQPLPTTLKKQEEKEILKSQLDSLLGLYHLLDWFAVDESNEVDPEF |
| | SARLTGIKLEMEPSLSFYNKARNYATKKPYSVEKFKLNFQMPTLASGWDVNKEKNNGAILFV |
| | KNGLYYLGIMPKQKGRYKALSFEPTEKTSEGFDKMYYDYFPDAAKMIPKCSTQLKAVTAHFQ |
| | THTTPILLSNNFIEPLEITKEIYDLNNPEKEPKKFQTAYAKKTGDQKGYREALCKWIDFTRD |
| | FLSKYTKTTSIDLSSLRPSSQYKDLGEYYAELNPLLYHISFQRIAEKEIMDAVETGKLYLFQ |
| | IYNKDFAKGHHGKPNLHTLYWTGLFSPENLAKTSIKLNGQAELFYRPKSRMKRMAHRLGEKM |
| | LNKKLKDQKTPIPDTLYQELYDYVNHRLSHDLSDEARALLPNVITKEVSHEIIKDRRFTSDK |
| | FFFHVPITLNYQAANSPSKFNQRVNAYLKEHPETPIIGIDRGERNLIYITVIDSTGKILEQR |
| | SLNTIQQFDYQKKLDNREKERVAARQAWSVVGTIKDLKQGYLSQVIHEIVDLMIHYQAVVVL |
| | ENLNFGFKSKRTGIAEKAVYQQFEKMLIDKLNCLVLKDYPAEKVGGVLNPYQLTDQFTSFAK |
| | MGTQSGFLFYVPAPYTSKIDPLTGFVDPFVWKTIKNHESRKHFLEGFDFLHYDVKTGDFILH |
| | FKMNRNLSFQRGLPGFMPAWDIVFEKNETQFDAKGTPFIAGKRIVPVIENHRFTGRYRDLYP |
| | ANELIALLEEKGIVFRDGSNILPKLLENDDSHAIDTMVALIRSVLQMRNSNAATGEDYINSP |
| | VRDLNGVCFDSRFQNPEWPMDADANGAYHIALKGQLLLNHLKESKDLKLQNGISNQDWLAYI |
| | QELRNPKKKRKVGGGSHHHHHH Stop (SEQ ID NO: 172) |

### DISCUSSION

In the current study we describe a novel method for precise genome editing using an AsCas12a-generated DSB, and a dsDNA repair template containing a matching 5′ overhang and a short double-stranded homologous arm. We called this method LAHR, for ‘Ligation-Assisted Homologous Recombination’. LAHR was the first precise genome editing tool that deployed both HDR and MMEJ mechanisms to repair an AsCas12a-generated DSB and introduced a desired nucleotide substitution. The complementary 5′ overhangs created by AsCas12a at the target site in the genome lock the repair template in place and ligate via a resection-independent MMEJ pathway, while template integration is completed by HDR. As summarized in FIG. 5, these two processes elegantly work together. The AsCas12a-cleaved genomic DSB end and repair template both contain homologous 5′ overhangs, such that they enter the MMEJ pathway at the level of Polyθ, skipping the need of strand resection. The remaining homologous arm of the template recombines by HDR. Finally, the base mismatch created by the template mutation is resolved, potentially by base-excision repair.

In Cas12a-mediated genome editing, a LAHR template is more efficient than a ssODN template in introducing a specific mutation, The comparison between LAHR and SpCas9-mediated HDR (using ssODN templates) is difficult, if not impossible, due to differences in PAM sites, cut-sites and repair template preference between Cas12a and Cas9 gene editing systems. Yet our data demonstrates that LAHR repair efficiency is on par with Cas9-mediated HDR.

We also noticed that the distance between the mutation and the AsCas12a target site affects LAHR editing efficiency, which is consistent with previous reports describing the effect of the distance between the mutation and nuclease target site upon Cas9 targeting and ssODN-mediated HDR. When the cut site is more than 10 bp removed from the Cas9 target site, HDR efficiency was shown to drop sharply (52). A solution to prevent this drop in efficiency is to extend the size of the flanking homologous arms on the ssODN (53). This principle may also apply in LAHR, but using simultaneous transduction of the AsCas12a RNP and the LAHR template DNA, we observed that repair efficiencies drop with LAHR templates over 100 bp in size, likely because these have more trouble passing the nuclear envelope. We determined that with an 80-bp homologous arm, a favorable distance between the mutation and the Cas12a target site is between 0-20 bp.

Taken together, we believe LAHR adds an attractive tool to the CRISPR toolbox and provides an essential alternative to traditional Cas9-mediated HDR particularly in circumstances where the Cas9-mediated editing is impaired by the lack of a suitable PAM site or efficient guide RNA candidates.

### REFERENCES

1. Urnov, F. D., Miller, J. C., Lee, Y.-L., Beausejour, C. M., Rock, J. M., Augustus, S., Jamieson, A. C., Porteus, M. H., Gregory, P. D. and Holmes, M. C. (2005) Highly efficient endogenous human gene correction using designed zinc-finger nucleases. Nature, 435, 646-651.
2. Moscou, M. J. and Bogdanove, A. J. (2009) A simple cipher governs DNA recognition by TAL effectors. Science, 326, 1501-1501.
3. Stoddard, B. L. (2005) Homing endonuclease structure and function. Quarterly reviews of biophysics, 38, 49.
4. Jinek, M., Chylinski, K., Fonfara, I., Hauer, M., Doudna, J. A. and Charpentier, E. (2012) A programmable dual-RNA-guided DNA endonuclease in adaptive bacterial immunity. Science, 337, 816-821.
5. Nelson, C. E., Hakim, C. H., Ousterout, D. G., Thakore, P. I., Moreb, E. A., Rivera, R. M. C., Madhavan, S., Pan, X., Ran, F. A. and Yan, W. X. (2016) In vivo genome editing improves muscle function in a mouse model of Duchenne muscular dystrophy. Science, 351, 403-407.
6. Wu, Y., Liang, D., Wang, Y., Bai, M., Tang, W., Bao, S., Yan, Z., Li, D. and Li, J. (2013) Correction of a genetic disease in mouse via use of CRISPR-Cas9. Cell Stem Cell, 13, 659-662.
7. Xie, F., Ye, L., Chang, J. C., Beyer, A. I., Wang, J., Muench, M. O. and Kan, Y. W. (2014) Seamless gene correction of p-thalassemia mutations in patient-specific iPSCs using CRISPR/Cas9 and piggyBac. Genome research, 24, 1526-1533.
8. Maddalo, D., Manchado, E., Concepcion, C. P., Bonetti, C., Vidigal, J. A., Han, Y.-C., Ogrodowski, P., Crippa, A., Rekhtman, N. and de Stanchina, E. (2014) In vivo engineering of oncogenic chromosomal rearrangements with the CRISPR/Cas9 system. Nature, 516, 423-427.
9. Cong, L., Ran, F. A., Cox, D., Lin, S., Barretto, R., Habib, N., Hsu, P. D., Wu, X., Jiang, W. and Marraffini, L. A. (2013) Multiplex genome engineering using CRISPR/Cas systems. Science, 339, 819-823.
10. Mali, P., Yang, L., Esvelt, K. M., Aach, J., Guell, M., DiCarlo, J. E., Norville, J. E. and Church, G. M. (2013) RNA-guided human genome engineering via Cas9. Science, 339, 823-826.
11. Pickar-Oliver, A. and Gersbach, C. A. (2019) The next generation of CRISPR-Cas technologies and applications. Nature reviews Molecular cell biology, 20, 490-507.
12. Knott, G. J. and Doudna, J. A. (2018) CRISPR-Cas guides the future of genetic engineering. Science, 361, 866-869.
13. Chen, X., Rinsma, M., Janssen, J. M., Liu, J., Maggio, I. and Gongalves, M. A. (2016) Probing the impact of chromatin conformation on genome editing tools. Nucleic Acids Res., 44, 6482-6492.
14. Adli, M. (2018) The CRISPR tool kit for genome editing and beyond. Nat. Commun., 9, 1-13.
15. Koonin, E. V., Makarova, K. S. and Zhang, F. (2017) Diversity, classification and evolution of CRISPR-Cas systems. Curr. Opin. Microbiol., 37, 67-78.
16. Zetsche, B., Gootenberg, J. S., Abudayyeh, O. O., Slaymaker, I. M., Makarova, K. S., Essletzbichler, P., Volz, S. E., Joung, J., van der Oost, J., Regev, A. et al. (2015) Cpf1 is a single RNA-guided endonuclease of a class 2 CRISPR-Cas system. Cell, 163, 759-771.
17. Swarts, D. C. and Jinek, M. (2018) Cas9 versus Cas12a/Cpf1: Structure-function comparisons and implications for genome editing. Wiley Interdiscip. Rev. RNA, 9, e1481.
18. Fonfara, I., Richter, H., Bratovic, M., Le Rhun, A. and Charpentier, E. (2016) The CRISPR-associated DNA-cleaving enzyme Cpf1 also processes precursor CRISPR RNA. Nature, 532, 517-521.
19. Stella, S., Mesa, P., Thomsen, J., Paul, B., Alcon, P., Jensen, S. B., Saligram, B., Moses, M. E., Hatzakis, N. S. and Montoya, G. (2018) Conformational activation promotes CRISPR-Cas12a catalysis and resetting of the endonuclease activity. Cell, 175, 1856-1871. e1821.
20. Yamano, T., Nishimasu, H., Zetsche, B., Hirano, H., Slaymaker, I. M., Li, Y., Fedorova, I., Nakane, T., Makarova, K. S., Koonin, E. V. et al. (2016) Crystal Structure of Cpf1 in Complex with Guide RNA and Target DNA. Cell, 165, 949-962.
21. Nishimasu, H., Ran, F. A., Hsu, P. D., Konermann, S., Shehata, S. I., Dohmae, N., Ishitani, R., Zhang, F. and Nureki, O. (2014) Crystal structure of Cas9 in complex with guide RNA and target DNA. Cell, 156, 935-949.
22. Jinek, M., Jiang, F., Taylor, D. W., Sternberg, S. H., Kaya, E., Ma, E., Anders, C., Hauer, M., Zhou, K. and Lin, S. (2014) Structures of Cas9 endonucleases reveal RNA-mediated conformational activation. Science, 343.
23. Zhang, Y., Long, C., Li, H., McAnally, J. R., Baskin, K. K., Shelton, J. M., Bassel-Duby, R. and Olson, E. N. (2017) CRISPR-Cpf1 correction of muscular dystrophy mutations in human cardiomyocytes and mice. Sci. Adv., 3, el 602814.
24. Ledford, H. (2015) Alternative CRISPR system could improve genome editing. Nature, 526, 17.
25. Zetsche, B., Heidenreich, M., Mohanraju, P., Fedorova, I., Kneppers, J., DeGennaro, E. M., Winblad, N., Choudhury, S. R., Abudayyeh, O. O. and Gootenberg, J. S. (2017) Multiplex gene editing by CRISPR-Cpf1 using a single crRNA array. Nat. Biotechnol., 35, 31-34.
26. Li, X., Wang, Y., Liu, Y., Yang, B., Wang, X., Wei, J., Lu, Z., Zhang, Y., Wu, J. and Huang, X. (2018) Base editing with a Cpf1-cytidine deaminase fusion. Nat. Biotechnol., 36, 324.
27. Kleinstiver, B. P., Sousa, A. A., Walton, R. T., Tak, Y. E., Hsu, J. Y., Clement, K., Welch, M. M., Horng, J. E., Malagon-Lopez, J. and Scarfò, I. (2019) Engineered CRISPR-Cas12a variants with increased activities and improved targeting ranges for gene, epigenetic and base editing. Nat. Biotechnol., 37, 276-282.
28. Chen, J. S., Ma, E., Harrington, L. B., Da Costa, M., Tian, X., Palefsky, J. M. and Doudna, J. A. (2018) CRISPR-Cas12a target binding unleashes indiscriminate single-stranded DNase activity. Science, 360, 436-439.
29. Li, S.-Y., Zhao, G.-P. and Wang, J. (2016) C-Brick: a new standard for assembly of biological parts using Cpf1. ACS Synth. Biol., 5, 1383-1388.
30. Lei, C., Li, S.-Y., Liu, J.-K., Zheng, X., Zhao, G.-P. and Wang, J. (2017) The CCTL (Cpf1-assisted Cutting and Taq DNA ligase-assisted Ligation) method for efficient editing of large DNA constructs in vitro. Nucleic Acids Res., 45, e74-e74.
31. Li, P., Zhang, L., Li, Z., Xu, C., Du, X. and Wu, S. (2019) Cas12a mediates efficient and precise endogenous gene tagging via MITI: microhomology-dependent targeted integrations. Cell. Mol. Life Sci., 1-10.
32. Kotecki, M., Reddy, P. S. and Cochran, B. H. (1999) Isolation and characterization of a near-haploid human cell line. Exp. Cell Res., 252, 273-280.
33. D'Astolfo, D. S., Pagliero, R. J., Pras, A., Karthaus, W. R., Clevers, H., Prasad, V., Lebbink, R. J., Rehmann, H. and Geijsen, N. (2015) Efficient intracellular delivery of native proteins. Cell, 161, 674-690.
34. Samulski, R., Zhu, X., Xiao, X., Brook, J., Housman, D., Epstein, N. a. and Hunter, L. (1991) Targeted integration of adeno-associated virus (AAV) into human chromosome 19. EMBO J., 10, 3941-3950.
35. Schneider, C. A., Rasband, W. S. and Eliceiri, K. W. (2012) NIH Image to ImageJ: 25 years of image analysis. Nat. Methods, 9, 671-675.
36. Komor, A. C., Kim, Y. B., Packer, M. S., Zuris, J. A. and Liu, D. R. (2016) Programmable editing of a target base in genomic DNA without double-stranded DNA cleavage. Nature, 533, 420-424.
37. Clement, K., Rees, H., Canver, M. C., Gehrke, J. M., Farouni, R., Hsu, J. Y., Cole, M. A., Liu, D. R., Joung, J. K. and Bauer, D. E. (2019) CRISPResso2 provides accurate and rapid genome editing sequence analysis. Nat Biotechnol, 37, 224-226.
38. Heim, R. and Tsien, R. Y. (1996) Engineering green fluorescent protein for improved brightness, longer wavelengths and fluorescence resonance energy transfer. Curr Biol., 6, 178-182.
39. Zhu, Z., Verma, N., Gonzalez, F., Shi, Z.-D. and Huangfu, D. (2015) A CRISPR/Cas-mediated selection-free knockin strategy in human embryonic stem cells. Stem Cell Rep., 4, 1103-1111.
40. Wang, Y., Liu, K. I., Sutrisnoh, N. B., Srinivasan, H., Zhang, J., Li, J., Zhang, F., Lalith, C. R. J., Xing, H., Shanmugam, R. et al. (2018) Systematic evaluation of CRISPR-Cas systems reveals design principles for genome editing in human cells. Genome Biol., 19, 62.
41. Richardson, C. D., Ray, G. J., DeWitt, M. A., Curie, G. L. and Corn, J. E. (2016) Enhancing homology-directed genome editing by catalytically active and inactive CRISPR-Cas9 using asymmetric donor DNA. Nat Biotechnol., 34, 339-344.
42. Paquet, D., Kwart, D., Chen, A., Sproul, A., Jacob, S., Teo, S., Olsen, K. M., Gregg, A., Noggle, S. and Tessier-Lavigne, M. (2016) Efficient introduction of specific homozygous and heterozygous mutations using CRISPR/Cas9. Nature, 533, 125-129.
43. Semenova, E., Jore, M. M., Datsenko, K. A., Semenova, A., Westra, E. R., Wanner, B., Van Der Oost, J., Brouns, S. J. and Severinov, K. (2011) Interference by clustered regularly interspaced short palindromic repeat (CRISPR) RNA is governed by a seed sequence. Proceedings of the National Academy of Sciences, 108, 10098-10103.
44. Robert, I., Dantzer, F. and Reina-San-Martin, B. (2009) Parpi facilitates alternative NHEJ, whereas Parp2 suppresses IgH/c-myc translocations during immunoglobulin class switch recombination. Journal of Experimental Medicine, 206, 1047-1056.
45. Carvajal-Garcia, J., Cho, J.-E., Carvajal-Garcia, P., Feng, W., Wood, R. D., Sekelsky, J., Gupta, G. P., Roberts, S. A. and Ramsden, D. A. (2020) Mechanistic basis for microhomology identification and genome scarring by polymerase theta. PNAS, 117, 8476-8485.
46. Mortensen, U. H., Bendixen, C., Sunjevaric, I. and Rothstein, R. (1996) DNA strand annealing is promoted by the yeast Rad52 protein. Proceedings of the National Academy of Sciences, 93, 10729-10734.
47. Ivanov, E. L., Sugawara, N., Fishman-Lobell, J. and Haber, J. E. (1996) Genetic requirements for the single-strand annealing pathway of double-strand break repair in Saccharomyces cerevisiae. Genetics, 142, 693-704.
48. Yeh, C. D., Richardson, C. D. and Corn, J. E. (2019) Advances in genome editing through control of DNA repair pathways. Nat. Cell Biol., 21, 1468-1478.
49. Xie, A., Hartlerode, A., Stucki, M., Odate, S., Puget, N., Kwok, A., Nagaraju, G., Yan, C., Alt, F. W. and Chen, J. (2007) Distinct roles of chromatin-associated proteins MDC1 and 53BP1 in mammalian double-strand break repair. Mol. Cell, 28, 1045-1057.
50. Mari, P.-O., Florea, B. I., Persengiev, S. P., Verkaik, N. S., Bruggenwirth, H. T., Modesti, M., Giglia-Mari, G., Bezstarosti, K., Demmers, J. A. and Luider, T. M. (2006) Dynamic assembly of end-joining complexes requires interaction between Ku70/80 and XRCC4. PNAS, 103, 18597-18602.
51. Sfeir, A. and Symington, L. S. (2015) Microhomology-mediated end joining: a back-up survival mechanism or dedicated pathway?Trends in biochemical sciences, 40, 701-714.
52. O'Brien, A. R., Wilson, L. O., Burgio, G. and Bauer, D. C. (2019) Unlocking HDR-mediated nucleotide editing by identifying high-efficiency target sites using machine learning. Sci. Rep., 9, 1-10.
53. Okamoto, S., Amaishi, Y., Maki, I., Enoki, T. and Mineno, J. (2019) Highly efficient genome editing for single-base substitutions using optimized ssODNs with Cas9-RNPs. Sci. Rep., 9, 1-11.

1. A method of editing a nucleic acid, said method comprising:
cutting or cleaving the nucleic acid to be edited; and
contacting the cut nucleic acid with a nucleic acid repair template;
wherein the nucleic acid repair template comprises an end which matches a cut end of the nucleic acid to be edited and a sequence that is homologous to a sequence of nucleic acid to be edited.

2. The method of claim 1, wherein the nucleic acid to be edited is cut with a nuclease which creates staggered cuts.

3. The method of claim 1, wherein the nucleic acid to be edited is cut with a nuclease of the CRISPR Cas system.

4. The method of claim 1, wherein the nucleic acid to be edited is cut with a Cas12a nuclease.

5. The method of claim 1, wherein the nucleic acid to be edited is a genomic nucleic acid sequence.

6. The method of claim 1, wherein the nucleic acid to be edited comprises a protospacer adjacent motif (PAM) or PAM sequence.

7. The method of claim 1, wherein the nucleic acid to be edited comprises a T-rich PAM sequence.

8. The method of claim 1, wherein the nucleic acid to be edited comprises a Cas12a cleavage site.

9. The method of claim 1, wherein the nucleic acid to be edited comprises an error for correction.

10. The method of claim 1, wherein the repair template comprises a sticky end which matches a cut end of the nucleic acid to be edited.

11. The method of claim 10, wherein the sticky end which matches a cut end of the nucleic acid to be edited comprises an overhang sequence.

12. The method of claim 11, wherein the overhang sequence is a 5′-overhang sequence.

13. The method of claim 12, wherein the 5′-overhang sequence matches the sequence of the 5′-overhang of one of the nuclease cut ends of the nucleic acid to be edited.

14. The method of claim 13, wherein the 5′-overhang sequence matches the sequence of a distal 5′-overhang cut end of the nucleic acid to be edited.

15. The method of claim 13, wherein the 5′ overhang sequence matches the sequence of a distal 5′-overhang cut end generated by Cas12a.

16. The method of claim 10, wherein the other end of the repair template is blunt ended or sticky-ended.

17. The method of claim 1, wherein the sequence of the repair template that is homologous to a sequence of nucleic acid to be edited, is fully or partially double stranded.

18. The method of claim 1, wherein the sequence of the repair template that is homologous to a sequence of nucleic acid to be edited, comprises the sequence edit.

19. The method of claim 1, wherein the sequence of the repair template that is homologous to a sequence of nucleic acid to be edited, may comprises a sequence which differs from the sequence of all or part of a target region of the nucleic acid to be edited by the presence of:
(i) one or more additional nucleobase(s); and/or
(ii) the absence of one or more nucleobases present in the target region.

20. The method of claim 1, wherein the sequence of the repair template that is homologous to a sequence of nucleic acid to be edited comprises between about 15 and about 300 bases and/or base pairs.

21. The method of claim 1, wherein the sequence of the repair template that is homologous to a sequence of nucleic acid to be edited comprises about 40, about 60 or about 80 bases and/or base pairs.

22. The method of claim 1, wherein the method combines microhomology-mediated end joining (MMEJ) with homology directed repair (HDR).

23. A repair template for use in a genome editing method or in a method of editing a nucleic acid, said repair template comprising an end which matches a cut end of a cut (genomic) nucleic acid to be edited and a sequence which is homologous to a sequence of the (genomic) nucleic acid to be edited.


SEQUENCE LISTING (194 sequences)

SEQ ID NO: 1 - DNA, 19 bp, synthetic construct
  source 1..19: mol_type = other DNA
atggtgagcaagggcgagg

SEQ ID NO: 2 - DNA, 24 bp, synthetic construct
  source 1..24: mol_type = other DNA
ttacttgtacagctcgtccatgcc

SEQ ID NO: 3 - DNA, 21 bp, synthetic construct
  source 1..21: mol_type = other DNA
gctcagtctggtctatctgcc

SEQ ID NO: 4 - DNA, 18 bp, synthetic construct
  source 1..18: mol_type = other DNA
tgtccctagtggccccac

SEQ ID NO: 5 - DNA, 21 bp, synthetic construct
  source 1..21: mol_type = other DNA
ggattggtgacagaaaagccc

SEQ ID NO: 6 - DNA, 17 bp, synthetic construct
  source 1..17: mol_type = other DNA
tcccctgcttcttggcc

SEQ ID NO: 7 - DNA, 21 bp, synthetic construct
  source 1..21: mol_type = other DNA
atcgaattcgggaggtggtcc

SEQ ID NO: 8 - DNA, 18 bp, synthetic construct
  source 1..18: mol_type = other DNA
aggactccgcgcctacag

SEQ ID NO: 9 - DNA, 25 bp, synthetic construct
  source 1..25: mol_type = other DNA
aggagatataccatgacccagtttg

SEQ ID NO: 10 - DNA, 22 bp, synthetic construct
  source 1..22: mol_type = other DNA
gttagcagccggatccttaatg

SEQ ID NO: 11 - DNA, 21 bp, synthetic construct
  source 1..21: mol_type = other DNA
tggactttgtctccttccctg

SEQ ID NO: 12 - DNA, 23 bp, synthetic construct
  source 1..23: mol_type = other DNA
gtggatgaatactgccatttgtg

SEQ ID NO: 13 - DNA, 21 bp, synthetic construct
  source 1..21: mol_type = other DNA
atgatctgtgtgtgttggttt

SEQ ID NO: 14 - DNA, 19 bp, synthetic construct
  source 1..19: mol_type = other DNA
agacctgacccaaacccag

SEQ ID NO: 15 - DNA, 21 bp, synthetic construct
  source 1..21: mol_type = other DNA
tggactttgtctccttccctg

SEQ ID NO: 16 - DNA, 21 bp, synthetic construct
  source 1..21: mol_type = other DNA
tggggcttttctgtcaccaat

SEQ ID NO: 17 - DNA, 22 bp, synthetic construct
  source 1..22: mol_type = other DNA
gaagaatcgcaaaaccagcaag

SEQ ID NO: 18 - DNA, 22 bp, synthetic construct
  source 1..22: mol_type = other DNA
atcgaatggatctgtctctgtc

SEQ ID NO: 19 - DNA, 19 bp, synthetic construct
  source 1..19: mol_type = other DNA
attgccaccacctgtcagc

SEQ ID NO: 20 - DNA, 19 bp, synthetic construct
  source 1..19: mol_type = other DNA
gcagaatccaggtggcaac

SEQ ID NO: 21 - DNA, 728 bp, synthetic construct
  source 1..728: mol_type = other DNA
gtgaagcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaaattcatctgcaccaccggcaagctgcccgtgccctggcccaccctcgtgaccaccctggtgcagtgtttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 737 bp, synthetic construct
  source 1..737: mol_type = other DNA
gtgaagcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccactttggtgaccaccctgacctccggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 23 - RNA, 96 nt, synthetic construct
  source 1..96: mol_type = other RNA
ggggccactagggacaggatgttttagagctagaaatagcaagttaaaataaggctagtccgttatcaacttgaaaaagtggcaccgagtcggtgc

SEQ ID NO: 24 - RNA, 43 nt, synthetic construct
  source 1..43: mol_type = other RNA
taatttctactcttgtagatagggtcagcttgccgtaggtggc

SEQ ID NO: 25 - RNA, 43 nt, synthetic construct
  source 1..43: mol_type = other RNA
taatttctactcttgtagatagccgctaccccgaccacatgaa

SEQ ID NO: 26 - RNA, 43 nt, synthetic construct
  source 1..43: mol_type = other RNA
taatttctactcttgtagatgtgaccaccctgacctccggcgt

SEQ ID NO: 27 - RNA, 43 nt, synthetic construct
  source 1..43: mol_type = other RNA
taatttctactcttgtagatatccatccgacattgaagttgac

SEQ ID NO: 28 - RNA, 43 nt, synthetic construct
  source 1..43: mol_type = other RNA
taatttctactcttgtagatgtggacaaacacgagaggcgggg

SEQ ID NO: 29 - RNA, 43 nt, synthetic construct
  source 1..43: mol_type = other RNA
taatttctactcttgtagatagtagtgaggaatgctacgagga

SEQ ID NO: 30 - RNA, 96 nt, synthetic construct
  source 1..96: mol_type = other RNA
ttggtgaccaccctgacctcgttttagagctagaaatagcaagttaaaataaggctagtccgttatcaacttgaaaaagtggcaccgagtcggtgc

SEQ ID NO: 31 - RNA, 96 nt, synthetic construct
  source 1..96: mol_type = other RNA
gctgaagcactgcacgccgggttttagagctagaaatagcaagttaaaataaggctagtccgttatcaacttgaaaaagtggcaccgagtcggtgc

SEQ ID NO: 32 - RNA, 96 nt, synthetic construct
  source 1..96: mol_type = other RNA
gcggctgaagcactgcacgcgttttagagctagaaatagcaagttaaaataaggctagtccgttatcaacttgaaaaagtggcaccgagtcggtgc

SEQ ID NO: 33 - RNA, 96 nt, synthetic construct
  source 1..96: mol_type = other RNA
gaagttgacttactgaagaagttttagagctagaaatagcaagttaaaataaggctagtccgttatcaacttgaaaaagtggcaccgagtcggtgc

SEQ ID NO: 34 - RNA, 96 nt, synthetic construct
  source 1..96: mol_type = other RNA
cagtaagtcaacttcaatgtgttttagagctagaaatagcaagttaaaataaggctagtccgttatcaacttgaaaaagtggcaccgagtcggtgc

SEQ ID NO: 35 - RNA, 96 nt, synthetic construct
  source 1..96: mol_type = other RNA
aagtcaacttcaatgtcggagttttagagctagaaatagcaagttaaaataaggctagtccgttatcaacttgaaaaagtggcaccgagtcggtgc

SEQ ID NO: 36 - RNA, 96 nt, synthetic construct
  source 1..96: mol_type = other RNA
tggacaaacacgagaggcgggttttagagctagaaatagcaagttaaaataaggctagtccgttatcaacttgaaaaagtggcaccgagtcggtgc

SEQ ID NO: 37 - RNA, 96 nt, synthetic construct
  source 1..96: mol_type = other RNA
cagtagtaaggaatgctacggttttagagctagaaatagcaagttaaaataaggctagtccgttatcaacttgaaaaagtggcaccgagtcggtgc

SEQ ID NO: 38 - DNA, 123 bp, synthetic construct
  source 1..123: mol_type = other DNA
gccacctacggcaagctgaccctgaaattcatctgcaccaccggcaagctgcccgtgccctggcccaccctcgtgaccaccctgacctacggcgtgcagtgtttcagccgctaccccgaccac

SEQ ID NO: 39 - DNA, 123 bp, synthetic construct
  source 1..123: mol_type = other DNA
tcatgtggtcggggtagcggctgaaacactgcacgccgtaggtcagggtggtcacgagggtgggccagggcacgggcagcttgccggtggtgcagatgaatttcagggtcagcttgccgtagg

SEQ ID NO: 40 - DNA, 127 bp, synthetic construct
  source 1..127: mol_type = other DNA
gccacctacggcaagctgaccctgaaattcatctgcaccaccggcaagctgcccgtgccctggcccaccctcgtgaccaccctgacctacggcgtgcagtgtttcagccgctaccccgaccacatga

SEQ ID NO: 41 - DNA, 127 bp, synthetic construct
  source 1..127: mol_type = other DNA
tcatgtggtcggggtagcggctgaaacactgcacgccgtaggtcagggtggtcacgagggtgggccagggcacgggcagcttgccggtggtgcagatgaatttcagggtcagcttgccgtaggtggc

SEQ ID NO: 42 - DNA, 123 bp, synthetic construct
  source 1..123: mol_type = other DNA
gccacgtatgggaaattaacattaaagtttatatgtacgacgggaaaattaccagtaccatggccaacactagtcacaacgttaacctacggcgtacaatactttagtcggtatccagatcat

SEQ ID NO: 43 - DNA, 123 bp, synthetic construct
  source 1..123: mol_type = other DNA
tcatatgatctggataccgactaaagtattgtacgccgtaggttaacgttgtgactagtgttggccatggtactggtaattttcccgtcgtacatataaactttaatgttaatttcccatacg

SEQ ID NO: 44 - DNA, 20 bp, synthetic construct
  source 1..20: mol_type = other DNA
ggtgaccaccctgacctacg

SEQ ID NO: 45 - DNA, 24 bp, synthetic construct
  source 1..24: mol_type = other DNA
acgccgtaggtcagggtggtcacc

SEQ ID NO: 46 - DNA, 40 bp, synthetic construct
  source 1..40: mol_type = other DNA
cccgtgccctggcccactttggtgaccaccctgacctacg

SEQ ID NO: 47 - DNA, 44 bp, synthetic construct
  source 1..44: mol_type = other DNA
acgccgtaggtcagggtggtcaccaaagtgggccagggcacggg

SEQ ID NO: 48 - DNA, 60 bp, synthetic construct
  source 1..60: mol_type = other DNA
tctgcaccaccggcaagctgcccgtgccctggcccactttggtgaccaccctgacctacg

SEQ ID NO: 49 - DNA, 60 bp, synthetic construct
  source 1..60: mol_type = other DNA
cgtaggtcagggtggtcaccaaagtgggccagggcacgggcagcttgccggtggtgcaga

SEQ ID NO: 50 - DNA, 80 bp, synthetic construct
  source 1..80: mol_type = other DNA
caagctgaccctgaagttcatctgcaccaccggcaagctgcccgtgccctggcccactttggtgaccaccctgacctacg

SEQ ID NO: 51 - DNA, 84 bp, synthetic construct
  source 1..84: mol_type = other DNA
acgccgtaggtcagggtggtcaccaaagtgggccagggcacgggcagcttgccggtggtgcagatgaacttcagggtcagcttg

SEQ ID NO: 52 - DNA, 100 bp, synthetic construct
  source 1..100: mol_type = other DNA
gagggcgatgccacctacggcaagctgaccctgaagttcatctgcaccaccggcaagctgcccgtgccctggcccactttggtgaccaccctgacctacg

SEQ ID NO: 53 - DNA, 104 bp, synthetic construct
  source 1..104: mol_type = other DNA
acgccgtaggtcagggtggtcaccaaagtgggccagggcacgggcagcttgccggtggtgcagatgaacttcagggtcagcttgccgtaggtggcatcgccctc

SEQ ID NO: 54 - DNA, 120 bp, synthetic construct
  source 1..120: mol_type = other DNA
tcagcgtgtccggcgagggcgagggcgatgccacctacggcaagctgaccctgaagttcatctgcaccaccggcaagctgcccgtgccctggcccactttggtgaccaccctgacctacg

SEQ ID NO: 55 - DNA, 124 bp, synthetic construct
  source 1..124: mol_type = other DNA
acgccgtaggtcagggtggtcaccaaagtgggccagggcacgggcagcttgccggtggtgcagatgaacttcagggtcagcttgccgtaggtggcatcgccctcgccctcgccggacacgctga

SEQ ID NO: 56 - DNA, 160 bp, synthetic construct
  source 1..160: mol_type = other DNA
atcctggtcgagctggacggcgacgtaaacggccacaagttcagcgtgtccggcgagggcgagggcgatgccacctacggcaagctgaccctgaagttcatctgcaccaccggcaagctgcccgtgccctggcccactttggtgaccaccctgacctacg

SEQ ID NO: 57 - DNA, 164 bp, synthetic construct
  source 1..164: mol_type = other DNA
acgccgtaggtcagggtggtcaccaaagtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggat

SEQ ID NO: 58 - DNA, 200 bp, synthetic construct
  source 1..200: mol_type = other DNA
ggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccactttggtgaccaccctgacctacg

SEQ ID NO: 59 - DNA, 204 bp, synthetic construct
  source 1..204: mol_type = other DNA
acgccgtaggtcagggtggtcaccaaagtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcacc

SEQ ID NO: 60 - DNA, 80 bp, synthetic construct; identical to SEQ ID NO: 50
  source 1..80: mol_type = other DNA

SEQ ID NO: 61 - DNA, 84 bp, synthetic construct; identical to SEQ ID NO: 51
  source 1..84: mol_type = other DNA

SEQ ID NO: 62 - DNA, 79 bp, synthetic construct
  source 1..79: mol_type = other DNA
caagctgaccctgaagttcatctgcaccaccggcaagctgcccgtgccctggcccactttggtgaccaccctgacctac

SEQ ID NO: 63 - DNA, 84 bp, synthetic construct; identical to SEQ ID NO: 51
  source 1..84: mol_type = other DNA

SEQ ID NO: 64 - DNA, 80 bp, synthetic construct; identical to SEQ ID NO: 50
  source 1..80: mol_type = other DNA

SEQ ID NO: 65 - DNA, 83 bp, synthetic construct
  source 1..83: mol_type = other DNA
cgccgtaggtcagggtggtcaccaaagtgggccagggcacgggcagcttgccggtggtgcagatgaacttcagggtcagcttg

SEQ ID NO: 66 - DNA, 80 bp, synthetic construct; identical to SEQ ID NO: 50
  source 1..80: mol_type = other DNA

SEQ ID NO: 67 - DNA, 84 bp, synthetic construct
  source 1..84: mol_type = other DNA
actccgtaggtcagggtggtcaccaaagtgggccagggcacgggcagcttgccggtggtgcagatgaacttcagggtcagcttg

SEQ ID NO: 68 - DNA, 84 bp, synthetic construct
  source 1..84: mol_type = other DNA
caagctgaccctgaagttcatctgcaccaccggcaagctgcccgtgccctggcccactttggtgaccaccctgacctacggcgt

SEQ ID NO: 69 - DNA, 80 bp, synthetic construct
  source 1..80: mol_type = other DNA
cgtaggtcagggtggtcaccaaagtgggccagggcacgggcagcttgccggtggtgcagatgaacttcagggtcagcttg

SEQ ID NO: 70 - DNA, 80 bp, synthetic construct; identical to SEQ ID NO: 50
  source 1..80: mol_type = other DNA

SEQ ID NO: 71 - DNA, 80 bp, synthetic construct; identical to SEQ ID NO: 69
  source 1..80: mol_type = other DNA

SEQ ID NO: 72 - DNA, 84 bp, synthetic construct; identical to SEQ ID NO: 68
  source 1..84: mol_type = other DNA

SEQ ID NO: 73 - DNA, 84 bp, synthetic construct; identical to SEQ ID NO: 51
  source 1..84: mol_type = other DNA

SEQ ID NO: 74 - DNA, 88 bp, synthetic construct
  source 1..88: mol_type = other DNA
caagctgaccctgaagttcatctgcaccaccggcaagctgcccgtgccctggcccactttggtgaccaccctgacctacggcgtgcag

SEQ ID NO: 75 - DNA, 84 bp, synthetic construct; identical to SEQ ID NO: 51
  source 1..84: mol_type = other DNA

SEQ ID NO: 76 - DNA, 80 bp, synthetic construct; identical to SEQ ID NO: 50
  source 1..80: mol_type = other DNA

SEQ ID NO: 77 - DNA, 84 bp, synthetic construct; identical to SEQ ID NO: 51
  source 1..84: mol_type = other DNA

SEQ ID NO: 78 - DNA, 160 bp, synthetic construct
  source 1..160: mol_type = other DNA
cggcaagctgaccctgaagttcatctgcaccaccggcaagctgcccgtgccctggcccactttggtgaccaccctgacctacggcgtgcagtgcttcagccgctaccccgaccacatgaagcagcacgacttcttcaagtccgccatgcccgaaggctac

SEQ ID NO: 79 - DNA, 160 bp, synthetic construct
  source 1..160: mol_type = other DNA
gtagccttcgggcatggcggacttgaagaagtcgtgctgcttcatgtggtcggggtagcggctgaagcactgcacgccgtaggtcagggtggtcaccaaagtgggccagggcacgggcagcttgccggtggtgcagatgaacttcagggtcagcttgccg

SEQ ID NO: 80 - DNA, 160 bp, synthetic construct; identical to SEQ ID NO: 78
  source 1..160: mol_type = other DNA

SEQ ID NO: 81 - DNA, 50 bp, synthetic construct
  source 1..50: mol_type = other DNA
cggcaagctgcccgtgccctggcccactttggtgaccaccctgacctacg

SEQ ID NO: 82 - DNA, 54 bp, synthetic construct
  source 1..54: mol_type = other DNA
acgccgtaggtcagggtggtcaccaaagtgggccagggcacgggcagcttgccg

SEQ ID NO: 83 - DNA, 101 bp, synthetic construct
  source 1..101: mol_type = other DNA
caccggcaagctgcccgtgccctggcccactttggtgaccaccctgacctacggcgtgcagtgcttcagccgctaccccgaccacatgaagcagcacgact

SEQ ID NO: 84 - DNA, 101 bp, synthetic construct
  source 1..101: mol_type = other DNA
agtcgtgctgcttcatgtggtcggggtagcggctgaagcactgcacgccgtaggtcagggtggtcaccaaagtgggccagggcacgggcagcttgccggtg

SEQ ID NO: 85 - DNA, 80 bp, synthetic construct; identical to SEQ ID NO: 50
  source 1..80: mol_type = other DNA

SEQ ID NO: 86 - DNA, 84 bp, synthetic construct; identical to SEQ ID NO: 51
  source 1..84: mol_type = other DNA

SEQ ID NO: 87 - DNA, 80 bp, synthetic construct
  source 1..80: mol_type = other DNA
caagctgaccctgaagttcatctgcaccaccggcaagctgcccgtgccctggcccactttggtgaccacactgacctacg

SEQ ID NO: 88 - DNA, 84 bp, synthetic construct
  source 1..84: mol_type = other DNA
acgccgtaggtcagtgtggtcaccaaagtgggccagggcacgggcagcttgccggtggtgcagatgaacttcagggtcagcttg

SEQ ID NO: 89 - DNA, 80 bp, synthetic construct
  source 1..80: mol_type = other DNA
caagctgaccctgaagttcatctgcaccaccggcaagctgcccgtgccctggcccacttaggtgaccaccctgacctacg

SEQ ID NO: 90 - DNA, 84 bp, synthetic construct
  source 1..84: mol_type = other DNA
acgccgtaggtcagggtggtcacctaagtgggccagggcacgggcagcttgccggtggtgcagatgaacttcagggtcagcttg

SEQ ID NO: 91 - DNA, 80 bp, synthetic construct
  source 1..80: mol_type = other DNA
caagctgaccctgaagttcatctgcaccaccggcaagctgcccgtgcccttgcccactttggtgaccaccctgacctacg

SEQ ID NO: 92 - DNA, 84 bp, synthetic construct
  source 1..84: mol_type = other DNA
acgccgtaggtcagggtggtcaccaaagtgggcaagggcacgggcagcttgccggtggtgcagatgaacttcagggtcagcttg

SEQ ID NO: 93 - DNA, 80 bp, synthetic construct
  source 1..80: mol_type = other DNA
caagctgaccctgaagttcatctgcaccacaggcaagctgcccgtgccctggcccactttggtgaccaccctgacctacg

SEQ ID NO: 94 - DNA, 84 bp, synthetic construct
  source 1..84: mol_type = other DNA
acgccgtaggtcagggtggtcaccaaagtgggccagggcacgggcagcttgcctgtggtgcagatgaacttcagggtcagcttg

SEQ ID NO: 95 - DNA, 80 bp, synthetic construct
  source 1..80: mol_type = other DNA
caagctgaccctgaagttcatatgcaccaccggcaagctgcccgtgccctggcccactttggtgaccaccctgacctacg

SEQ ID NO: 96 - DNA, 84 bp, synthetic construct
  source 1..84: mol_type = other DNA
acgccgtaggtcagggtggtcaccaaagtgggccagggcacgggcagcttgccggtggtgcatatgaacttcagggtcagcttg

SEQ ID NO: 97 - DNA, 80 bp, synthetic construct
  source 1..80: mol_type = other DNA
caagctgactctgaagttcatctgcaccaccggcaagctgcccgtgccctggcccactttggtgaccaccctgacctacg

SEQ ID NO: 98 - DNA, 84 bp, synthetic construct
  source 1..84: mol_type = other DNA
acgccgtaggtcagggtggtcaccaaagtgggccagggcacgggcagcttgccggtggtgcagatgaacttcagagtcagcttg

SEQ ID NO: 99 - DNA, 80 bp, synthetic construct
  source 1..80: mol_type = other DNA
aaagctgaccctgaagttcatctgcaccaccggcaagctgcccgtgccctggcccactttggtgaccaccctgacctacg

SEQ ID NO: 100 - DNA, 84 bp, synthetic construct
  source 1..84: mol_type = other DNA
acgccgtaggtcagggtggtcaccaaagtgggccagggcacgggcagcttgccggtggtgcagatgaacttcagggtcagcttt

SEQ ID NO: 101 - DNA, 80 bp, synthetic construct
  source 1..80: mol_type = other DNA
tcacgtcatccagcagagaatggaaagtcaaatttcctgaattgctatgtgtctgggtttcatccatccgacattgaagt

SEQ ID NO: 102 - DNA, 84 bp, synthetic construct
  source 1..84: mol_type = other DNA
gtcaacttcaatgtcggatggatgaaacccagacacatagcaattcaggaaatttgactttccattctctgctggatgacgtga

SEQ ID NO: 103 - DNA, 100 bp, synthetic construct
  source 1..100: mol_type = other DNA
agtcaaatttcctgaattgctatgtgtctgggtttcatccatccgacattgaagttgacttactgaagaatggagagagaattgaaaaagtggagcattc

SEQ ID NO: 104 - DNA, 100 bp, synthetic construct
  source 1..100: mol_type = other DNA
gaatgctccactttttcaattctctctccattcttcagtaagtcaacttcaatgtcggatggatgaaacccagacacatagcaattcaggaaatttgact

SEQ ID NO: 105 - DNA, 84 bp, synthetic construct
  source 1..84: mol_type = other DNA
ccccgcttctcgtgtttgtccactaaatgtgacgcccaggctcaggacccccagctgcctcattattgtggcctgtttgactct

SEQ ID NO: 106 - DNA, 80 bp, synthetic construct
  source 1..80: mol_type = other DNA
agagtcaaacaggccacaataatgaggcagctgggggtcctgagcctgggcgtcacatttagtggacaaacacgagaagc

SEQ ID NO: 107 - DNA, 100 bp, synthetic construct
  source 1..100: mol_type = other DNA
agcaaagccatgttgagggtattactcctgagtgtgtatgttacccccgcttctcgtgtttgtccactaaatgtgacgcccaggctcaggacccccagct

SEQ ID NO: 108 - DNA, 80 bp, synthetic construct
  source 1..80: mol_type = other DNA
agacccagagatacatggctatttcagggacccccactgcttgggggagcaggagtatttcagtagtgaggaatgctgcg

SEQ ID NO: 109 - DNA, 84 bp, synthetic construct
  source 1..84: mol_type = other DNA
tcctcgcagcattcctcactactgaaatactcctgctcccccaagcagtgggggtccctgaaatagccatgtatctctgggtct

SEQ ID NO: 110 - DNA, 100 bp, synthetic construct
  source 1..100: mol_type = other DNA
aggagccagagcagctcacctgctccaggtgggcgagctgtcatcctcgcagcattcctcactactgaaatactcctgctcccccaagcagtgggggtcc

SEQ ID NO: 111 - RNA, 19 nt, synthetic construct
  source 1..19: mol_type = other RNA
gccaggttctagaggatga

SEQ ID NO: 112 - RNA, 21 nt, synthetic construct
  source 1..21: mol_type = other RNA
gcacaagaacttatggaaagt

SEQ ID NO: 113 - RNA, 19 nt, synthetic construct
  source 1..19: mol_type = other RNA
ggaagagatagtttgattt

SEQ ID NO: 114 - RNA, 19 nt, synthetic construct
  source 1..19: mol_type = other RNA
gacatatccttgttctaca

SEQ ID NO: 115 - RNA, 19 nt, synthetic construct
  source 1..19: mol_type = other RNA
gaagttctgtcacagctga

SEQ ID NO: 116 - RNA, 19 nt, synthetic construct
  source 1..19: mol_type = other RNA
gtcttcaagggtgtctgtc

SEQ ID NO: 117 - RNA, 19 nt, synthetic construct
  source 1..19: mol_type = other RNA
ccttaagactgtaggtact

SEQ ID NO: 118 - RNA, 21 nt, synthetic construct
  source 1..21: mol_type = other RNA
caaacaaacccttatcgtaaa

SEQ ID NO: 119 - RNA, 21 nt, synthetic construct
  source 1..21: mol_type = other RNA
aagcctccgctcctgaacaat

SEQ ID NO: 120 - RNA, 21 nt, synthetic construct
  source 1..21: mol_type = other RNA
aagatagagcgtgaaggcgaa

SEQ ID NO: 121 - RNA, 21 nt, synthetic construct
  source 1..21: mol_type = other RNA
aagccagacaaaagaggtgaa

SEQ ID NO: 122 - RNA, 19 nt, synthetic construct
  source 1..19: mol_type = other RNA
ccgtgaatatgattgctat

SEQ ID NO: 123 - RNA, 19 nt, synthetic construct
  source 1..19: mol_type = other RNA
gggaattagtgaagccaaa

SEQ ID NO: 124 - RNA, 19 nt, synthetic construct
  source 1..19: mol_type = other RNA
gaattgagactggatctat

SEQ ID NO: 125 - RNA, 19 nt, synthetic construct
  source 1..19: mol_type = other RNA
gggaattagtgaagccaaa

SEQ ID NO: 126 - RNA, 19 nt, synthetic construct
  source 1..19: mol_type = other RNA
agtccaaggctttatcttt

SEQ ID NO: 127 - DNA, 21 bp, synthetic construct
  source 1..21: mol_type = other DNA
agatggaccctactggaagtc

SEQ ID NO: 128 - DNA, 26 bp, synthetic construct
  source 1..26: mol_type = other DNA
tgttcattgaacccactattaccgtc

SEQ ID NO: 129 - DNA, 21 bp, synthetic construct
  source 1..21: mol_type = other DNA
agcaaggacatccctgtgaca

SEQ ID NO: 130 - DNA, 21 bp, synthetic construct
  source 1..21: mol_type = other DNA
acctctgaccagagagctgca

SEQ ID NO: 131 - DNA, 22 bp, synthetic construct
  source 1..22: mol_type = other DNA
caagaagagaaccttgaagcaa

SEQ ID NO: 132 - DNA, 21 bp, synthetic construct
  source 1..21: mol_type = other DNA
tggatgctcatgtcaaaaggt

SEQ ID NO: 133 - DNA, 21 bp, synthetic construct
  source 1..21: mol_type = other DNA
catcttccttgacttgatgca

SEQ ID NO: 134 - DNA, 21 bp, synthetic construct
  source 1..21: mol_type = other DNA
tagagctttattggaggaggc

SEQ ID NO: 135 - DNA, 23 bp, synthetic construct
  source 1..23: mol_type = other DNA
tcagaagagcagcgctttaacaa

SEQ ID NO: 136 - DNA, 20 bp, synthetic construct
  source 1..20: mol_type = other DNA
tcgtccacatcaccaccttc

SEQ ID NO: 137 - DNA, 20 bp, synthetic construct
  source 1..20: mol_type = other DNA
aagacttgcggcaatacatg

SEQ ID NO: 138 - DNA, 22 bp, synthetic construct
  source 1..22: mol_type = other DNA
cagcatattccaaatatgctgc

SEQ ID NO: 139 - DNA, 22 bp, synthetic construct
  source 1..22: mol_type = other DNA
caacagatggcaactgaaaatg

SEQ ID NO: 140 - DNA, 22 bp, synthetic construct
  source 1..22: mol_type = other DNA
cttgtttcaggaactggaagac

SEQ ID NO: 141 - DNA, 24 bp, synthetic construct
  source 1..24: mol_type = other DNA
catgacagagacagtgaagaattg

SEQ ID NO: 142 - DNA, 20 bp, synthetic construct
  source 1..20: mol_type = other DNA
actttggagcataccctctc

SEQ ID NO: 143 - DNA, 22 bp, synthetic construct
  source 1..22: mol_type = other DNA
atactccatcctcagtgaggtc

SEQ ID NO: 144 - DNA, 20 bp, synthetic construct
  source 1..20: mol_type = other DNA
atgggatccttgctgctatc

SEQ ID NO: 145 - DNA, 21 bp, synthetic construct
  source 1..21: mol_type = other DNA
gagattctgaagaagccgaga

SEQ ID NO: 146 - DNA, 22 bp, synthetic construct
  source 1..22: mol_type = other DNA
caacttctcccaacaggattaa

SEQ ID NO: 147 - DNA, 22 bp, synthetic construct
  source 1..22: mol_type = other DNA
caccaattccaggtagaataga

SEQ ID NO: 148 - DNA, 18 bp, synthetic construct
  source 1..18: mol_type = other DNA
acacagatggcttctcac

SEQ ID NO: 149 - DNA, 18 bp, synthetic construct
  source 1..18: mol_type = other DNA
atggcagcctcgctttat

SEQ ID NO: 150 - DNA, 20 bp, synthetic construct
  source 1..20: mol_type = other DNA
tgtggcgtcgaaacatactg

SEQ ID NO: 151 - DNA, 19 bp, synthetic construct
  source 1..19: mol_type = other DNA
agtgtggcataaatgccaa

SEQ ID NO: 152 - DNA, 18 bp, synthetic construct
  source 1..18: mol_type = other DNA
gtgaaacccattggaact

SEQ ID NO: 153 - DNA, 18 bp, synthetic construct
  source 1..18: mol_type = other DNA
agttccaatgggtttcac

SEQ ID NO: 154 - DNA, 16 bp, synthetic construct
  source 1..16: mol_type = other DNA
tggcaggtgacagcta

SEQ ID NO: 155 - DNA, 21 bp, synthetic construct
  source 1..21: mol_type = other DNA
acagcactcctgtaactgtct

SEQ ID NO: 156 - DNA, 19 bp, synthetic construct
  source 1..19: mol_type = other DNA
tgttgtgcgttggtcagcg

SEQ ID NO: 157 - DNA, 18 bp, synthetic construct
  source 1..18: mol_type = other DNA
gttgacctcaacaatggc

SEQ ID NO: 158 - DNA, 20 bp, synthetic construct
  source 1..20: mol_type = other DNA
ctggcgtggaagcttattta

SEQ ID NO: 159 - DNA, 21 bp, synthetic construct
  source 1..21: mol_type = other DNA
gcctcagacagtggttcaaag

SEQ ID NO: 160 - DNA, 24 bp, synthetic construct
  source 1..24: mol_type = other DNA
taacatatagacaaacgcacaccg

SEQ ID NO: 161 - DNA, 17 bp, synthetic construct
  source 1..17: mol_type = other DNA
tgagcaagggcgaggag

SEQ ID NO: 162 - DNA, 23 bp, synthetic construct
  source 1..23: mol_type = other DNA
gagggaaagataccaagtcacgg

SEQ ID NO: 163 - DNA, 24 bp, synthetic construct
  source 1..24: mol_type = other DNA
catcagtatctcagcaggtgccac

SEQ ID NO: 164 - DNA, 18 bp, synthetic construct
  source 1..18: mol_type = other DNA
tggaaatggaattgggag

SEQ ID NO: 165 - DNA, 39 bp, synthetic construct
  source 1..39: mol_type = other DNA
gatgtgtataagagacaggaccctgaagttcatctgcac

SEQ ID NO: 166 - DNA, 36 bp, synthetic construct
  source 1..36: mol_type = other DNA
cgtgtgctcttccgatctgctcctggacgtagcctt

SEQ ID NO: 167 - DNA, 35 bp, synthetic construct
  source 1..35: mol_type = other DNA
gatgtgtataagagacagaagaactggaagcccga

SEQ ID NO: 168 - DNA, 41 bp, synthetic construct
  source 1..41: mol_type = other DNA
cgtgtgctcttccgatctgtcaaacaggccacaataatgag

SEQ ID NO: 169 - DNA, 41 bp, synthetic construct
  source 1..41: mol_type = other DNA
gatgtgtataagagacaggacccagagatacatggctattt

SEQ ID NO: 170 - DNA, 31 bp, synthetic construct
  source 1..31: mol_type = other DNA
cgtgtgctcttccgatctagggcggcctccc

SEQ ID NO: 171 - protein, 1393 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSGTRLPKKKRKVGGGSHHHHHH

SEQ ID NO: 172 - protein, 1324 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVGGGSHHHHHH

SEQ ID NO: 173 - DNA, 23 bp, synthetic construct
  source 1..23: mol_type = other DNA
ggggccactagggacaggattgg

SEQ ID NO: 174 - DNA, 23 bp, synthetic construct
  source 1..23: mol_type = other DNA
ccaatcctgtccctagtggcccc

SEQ ID NO: 175 - DNA, 28 bp, synthetic construct
  source 1..28: mol_type = other DNA
tttggtgaccaccctgacctccggcgtg

SEQ ID NO: 176 - DNA, 28 bp, synthetic construct
  source 1..28: mol_type = other DNA
cacgccggaggtcagggtggtcaccaaa

SEQ ID NO: 177 - DNA, 23 bp, synthetic construct
  source 1..23: mol_type = other DNA
tttggtgaccaccctgacctacg

SEQ ID NO: 178 - DNA, 27 bp, synthetic construct
  source 1..27: mol_type = other DNA
acgccgtaggtcagggtggtcaccaaa

SEQ ID NO: 179 - DNA, 28 bp, synthetic construct
  source 1..28: mol_type = other DNA
tttggtgaccaccctgacctccggcgtg

SEQ ID NO: 180 - DNA, 28 bp, synthetic construct
  source 1..28: mol_type = other DNA
cacgccggaggtcagggtggtcaccaaa

SEQ ID NO: 181 - DNA, 23 bp, synthetic construct
  source 1..23: mol_type = other DNA
tctggtgaccaccctgacctacg

SEQ ID NO: 182 - DNA, 27 bp, synthetic construct
  source 1..27: mol_type = other DNA
acgccgtaggtcagggtggtcaccaga

SEQ ID NO: 183 - DNA, 23 bp, synthetic construct
  source 1..23: mol_type = other DNA
tttggttaccaccctgacctacg

SEQ ID NO: 184 - DNA, 27 bp, synthetic construct
  source 1..27: mol_type = other DNA
acgccgtaggtcagggtggtaaccaaa

SEQ ID NO: 185 - DNA, 23 bp, synthetic construct
  source 1..23: mol_type = other DNA
tctggttaccaccctgacctacg

SEQ ID NO: 186 - DNA, 27 bp, synthetic construct
  source 1..27: mol_type = other DNA
acgccgtaggtcagggtggtaaccaga

SEQ ID NO: 187 - DNA, 27 bp, synthetic construct
  source 1..27: mol_type = other DNA
tttggtgaccaccctgacctacggcgt

SEQ ID NO: 188 - DNA, 27 bp, synthetic construct
  source 1..27: mol_type = other DNA
tctggtgaccaccctgacctacggcgt

SEQ ID NO: 189 - DNA, 27 bp, synthetic construct
  source 1..27: mol_type = other DNA
tttggttaccaccctgacctacggcgt

SEQ ID NO: 190 - DNA, 27 bp, synthetic construct
  source 1..27: mol_type = other DNA
tctggttaccaccctgacctacggcgt

SEQ ID NO: 191 - DNA, 38 bp, synthetic construct
  source 1..38: mol_type = other DNA
atgttacccccgcctctcgtgtttgtccactaaatgtg

SEQ ID NO: 192 - DNA, 38 bp, synthetic construct
  source 1..38: mol_type = other DNA
cacatttagtggacaaacacgagaggcgggggtaacat

SEQ ID NO: 193 - DNA, 37 bp, synthetic construct
  source 1..37: mol_type = other DNA
gagtatttcagtagtgaggaatgctacgaggatgaca

SEQ ID NO: 194 - DNA, 37 bp, synthetic construct
  source 1..37: mol_type = other DNA
tgtcatcctcgtagcattcctcactactgaaatactc